(19) United States
(12) Patent Application Publication
(10) Pub. No.: US 2016/0068609 A1
(43) Pub. Date: Mar. 10, 2016

(54) ANTI-CANCER TREATMENTS WITH ANTI-EGFR ANTIBODIES HAVING A LOW FUCOSYLATION

(71) Applicant: GLYCOTOPE GMBH, Berlin (DE)

(72) Inventors: Steffen GOLETZ, Berlin (DE); Antje DANIELCZYK, Berlin (DE)

(21) Appl. No.: 14/784,470

(22) PCT Filed: Apr. 22, 2014
(86) PCT No.: PCT/EP2014/058118

§ 371 Date: Oct. 14, 2015

(30) Foreign Application Priority Data
Apr. 22, 2013 (EP) 13002106.6
Apr. 22, 2013 (EP) 13002108.2

(51) Int. Cl. C07K 16/40; A61N 5/10; A61K 47/48; A61K 45/06; A61K 39/395

(52) U.S. Cl. CPC C07K 16/40; A61K 45/06; A61K 39/39558; A61K 47/48646; A61N 5/10; C07K 2317/732; C07K 2317/41; C07K 2317/76; A61K 2039/505

(57) ABSTRACT

The present invention pertains to the field of cancer therapy using anti-cancer antibodies. The medical use of anti-EGFR antibodies having improved glycosylation characteristics, in particular a reduced fucosylation, is provided which show anti-cancer efficacy and an improved adverse side effect profile.

### FIELD OF THE INVENTION

The present invention pertains to novel medical uses of anti-EGFR antibodies having improved characteristics in anti-cancer treatments. Said anti-EGFR antibodies show superior therapeutic efficacy with a greatly reduced occurrence of adverse side effects such as skin reactions. Hence, the treatment of cancer patients with these antibodies is well tolerated and it is possible to treat patients which otherwise could not be treated due to severe adverse reactions caused by conventional anti-EGFR antibodies.

### BACKGROUND OF THE INVENTION

Antibodies are widely used agents in the field of medicine and research. In medicine, they find application in many different fields, in particular as therapeutic agents in the treatment and prophylaxis of a variety of diseases, in particular neoplastic diseases such as cancer. However, therapeutic results obtained by antibody therapy of cancer patients are highly variable. A significant percentage of the therapies using anti-cancer antibodies shows no or only a small alleviation of the disease and sometimes are limited to specific patient groups.

Exemplary established anti-cancer antibodies are antibodies against the epidermal growth factor receptor (EGFR). The EGF receptor is a receptor tyrosine kinase which is anchored in the plasma membrane. Binding of the ligands epidermal growth factor (EGF) or transforming growth factor alpha (TGFα) to the extracellular domain of the EGFR results in dimerization of the receptor and stimulation of its intracellular protein-tyrosine kinase activity. The signal transduction cascades initiated by the active receptor dimer control cell migration, adhesion, and proliferation. The human epidermal growth factor receptor (EGFR) protein is thought to be a unique and useful target for antibody therapy against cancers comprising high EGFR activity, either by EGFR overexpression or by mutations in the EGFR protein. EGFR is over-expressed in several cancers, including but not limited to colorectal cancer, lung cancer, pancreatic cancer and head-and-neck cancer. Mutations, amplifications or misregulations of EGFR or family members are implicated in about 30% of all epithelial cancers and are associated with a poor prognosis.

Several antibodies directed against EGFR are known in the art. Some of them are already approved for medical applications. For example, the recombinant chimeric mouse/human IgG1 anti-EGFR monoclonal antibody cetuximab (Erbitux®, Merck) was approved for clinical use in Europe and the United States for the treatment of metastatic colorectal cancer and squamous cell cancer of the head and neck. Cetuximab is used as mono- and combination therapy. Cetuximab is expressed in SP2/0 cells (murine cell line) and therefore is highly fucosylated and has an overall murine glycosylation pattern. Another anti-EGFR antibody approved for medical applications is the human monoclonal IgG2 antibody panitumumab (Vectibix®, Amgen).

These anti-EGFR antibodies are effective in cancer treatment because they are able to inhibit EGFR signaling. They bind to the extracellular domain of EGFR and prevent binding of the natural activating ligands such as EGF and TGFα, thereby inhibiting dimerization and activation of EGFR and its downstream signaling cascade. It is to be noted that this mechanism of action is only relevant for tumors which depend on the activation of EGFR for proliferation. Especially in colorectal cancer, a large portion of the tumors, however, comprise a mutation in the Kirsten Ras gene (KRAS), rendering the K-Ras protein constantly active. K-Ras is an important member of the downstream signaling cascade of EGFR and an inhibition of EGFR signaling will generally have no effect on tumors wherein K-Ras is constantly active. Because of this, cetuximab and panitumumab are only approved for the treatment of KRAS wild-type metastatic colorectal cancer.

As a further mode of action, the IgG1 antibody cetuximab also induces antibody-dependent cell-mediated cytotoxicity (ADCC). The antibody bound to its antigen on the surface of the target cancer cell recruits immune cells which then destroy the cancer cell. This is mediated by binding of the constant region of the antibody to Fcγ receptors on the immune cells. In particular, the Fc region of IgG1 antibodies binds to FcγRIIIa on natural killer cells.

Reduced fucose, including afucosylated IgG1 antibodies have been shown to have enhanced antibody-dependent cell-mediated cytotoxicity (ADCC) and therefore provide an opportunity for development of biobetter antibodies. Evidence suggests that the absence of fucose from the primary N-acetylglucosamine results in increased affinity of binding of IgG1 antibodies to the FcγRIIIa receptor with consequently increased ADCC efficacy, mediated by natural killer (NK) cells. This is confirmed in studies employing non-fucosylated glycoforms produced in mutant CHO cells that are deficient in addition of the fucose residue, in particular CHO cells in which the α(1-6) fucosyltransferase enzyme has been knocked out. The affinity of the non-fucosylated IgG1 glycoform for FcγRI or the C1 component of complement was reported to be unaffected; a small increase in affinity for FcγRIIa and FcγRIIb was reported, but as the activating/inhibitory ratio was maintained, it was concluded that it would not be functionally significant. The enhanced ADCC observed for afucosylated IgG-Fc results, in part, from the increased affinity for FcγRIIIa, overcoming competition of normal serum IgG. Improved ADCC was also presented for afucosylated cetuximab. The FcγRIIIa receptor is polymorphic and it has been shown that the FcγRIIIa-158V (valine) form has a higher affinity for fucosylated IgG1 than the FcγRIIIa-158F (phenylalanine) form. It was demonstrated in vitro that fucosylated IgG1 antibody is more efficient at mediating ADCC through homozygous FcγRIIIa-158V bearing cells than through homozygous FcγRIIIa-158F or heterozygous FcγRIIIa-158V/FcγRIIIa-158F cells. It was anticipated, therefore, that similar differences in ADCC efficacy might pertain in vivo, depending on the polymorphic form of FcγRIIIa expressed. For example, Zhang et al. (2007) J Clin Oncol 25, 3712-3718 describes that the allelic status of FcγRIIIa influences the average time of progression-free survival of colorectal cancer patients receiving cetuximab monotherapy. The use of afucosylated antibodies for treating respective subpopulations of weak-responder patients which are F/F homozygous or V/F heterozygous is suggested in the prior art, for example US 2006/0182741. Afucosylated antibodies and antibodies with a reduced fucose content are also described in EP 1 500 400 and WO 2008/028686.

However, while for several types of cancer good therapeutic results were obtained with anti-EGFR antibodies, kidney cancer, in particular renal cell carcinomas, so far often evade antibody therapy as well as many other anti-cancer therapies. It is a general observation that kidney cancers often do not respond to antibody therapy, chemotherapy or radiotherapy. This in particular applies to EGFR targeting cancer treatment, although 70% to 90% of renal cell carcinomas express EGFR. For example, a phase II clinical study with cetuximab (Erbitux®) did not show any partial or complete response and only a very short median time to progression was observed (see Motzer, R. J. et al. (2003) Investigational New Drugs 21, 99-101). Likewise, also the EGFR inhibitor ZD1839, a potent tyrosine kinase inhibitor, did not show therapeutic efficacy in a phase II clinical trial (see Drucker, B. J. et al. (2002) Proceedings of the American Society of Clinical Oncology 21, abstract 720). Therefore, patients with kidney cancer have a very poor prognosis. Due to this lack of response, kidney cancers, in particular renal cell carcinomas, are known to be the most lethal of all the genitourinary tumors. Standard therapy today is the thorough surgical removal of the tumor mass. However, metastatic renal cell carcinoma presents a special challenge to oncologists, as about 70% of the patients develop metastases during the course of their disease. The 5 year survival for patients with metastatic renal cell carcinoma is as low as 5 to 15%. This is mainly based on the fact that there is currently no established adjuvant therapy for renal cell carcinoma after surgical excision of the primary tumor and visible metastases. The use of non-specific cytokines has so far been shown to be ineffective. Unlike most other cancers, renal cell carcinoma is resistant to most cytotoxic and cytostatic agents, which severely limits possible effective adjuvant therapy. Trials of cancer vaccines, radiotherapy, chemotherapy, immunotherapy, or biologic therapies have been met with little success, even if they were based on promising in vitro data. Therefore, currently the standard of care for completely resected high-risk renal cell carcinoma is close observation with no other therapy.

In one clinical study, however, some results were obtained using the anti-EGFR antibody panitumumab (ABX-EGF of Amgen Inc.) (see Rowinsky, E. K. et al. (2004) Journal of Clinical Oncology 22, 3003-3015). Some responses were observed and about 50% of the patients had a stable disease for at least 8 weeks. The median progression-free survival was 100 days. However, the treatment was associated with a very high incidence of adverse skin reactions. Depending on the dosage level, up to 100% of the patients suffered under acneiform rash. Furthermore, panitumumab is an IgG2 antibody which is not capable of inducing ADCC in the patient. Hence, tumors which are independent of EGFR signaling such as those with a KRAS mutation are not susceptible to a therapy with panitumumab.

Therefore, there is a great need for novel therapies in order to treat kidney cancer such as clear cell renal cancer and also non-clear cell renal cancers.

A general observation with all anti-EGFR antibodies such as cetuximab and also other EGFR inhibitors is that they frequently cause adverse skin reactions. These skin reactions range from mild skin irritations to severe skin rashes covering the majority of the patient's body surface. These skin rashes are accompanied by pustules, itching, swelling and often pain and in severe cases may be associated with ulcerations, infections and even necrosis of skin areas. Skin rashes, in particular acneiform rash, are highly common for patients treated with EGFR inhibitors, in particular anti-EGFR antibodies. For example, about 76% to 88% of the patients treated with the anti-EGFR antibody cetuximab (Erbitux®) are affected by acneiform rash, including up to 17% having a severe form of this adverse reaction (see, e.g., the prescribing information for Erbitux of Eli Lilly & Co.). These adverse skin reactions also occur with other anti-EGFR antibodies such as panitumumab. Therefore, it is widely described in the literature that the adverse skin reactions, also referred to as skin toxicity, is a unique class-specific toxicity of all EGFR inhibitors and in particular occur with anti-EGFR antibodies (see e.g. Li, T. and Perez-Soler, R. (2009) Targeted Oncology 4, 107-119 and Perez-Soler, R. et al. (2005) The Oncologist 10, 345-356). Because the adverse skin reactions are so common and in view of experimental data obtained from several clinical studies with different EGFR inhibitors including different anti-EGFR antibodies, it is believed up to today that the occurrence of adverse skin reaction is even directly correlated with the efficacy of the EGFR inhibitor and hence, with the success of the cancer treatment. Therefore, the occurrence of skin reactions was believed to be a marker for the therapeutic efficacy. For example, Fracasso, P. M. et al. (2007) Clinical Cancer Research 13, 986-993 stated that in their clinical study as well as in other studies, patients with a partial response or a stable disease due to treatment with the anti-EGFR antibody cetuximab (Erbitux®) had a statistically significant higher overall grade of rash than those with progression of disease. Also according to Li, T. and Perez-Soler, R. (2009) Targeted Oncology 4, 107-119, the presence and severity of skin rash is associated with improved clinical efficacy in patients receiving EGFR inhibitors. Also in a further phase I clinical study using the monoclonal IgG2 anti-EGFR antibody ABX-EGF (panitumumab), about 80% of the patients suffered under acneiform skin rash, including significant numbers of severe cases (see Rowinsky, E. K. et al. (2004) Journal of Clinical Oncology 22, 3003-3015). In both studies, the adverse skin reactions were again considered as potential marker of the drug action and as potential surrogate marker of clinical benefit (see also Perez-Soler et al, 2005).

Also in a recent phase I clinical study with the glycoengineered monoclonal anti-EGFR antibody RG7160 (GA201) 80% of the patients developed a skin rash, with 25% of the patients having a severe rash of grade 3 or even of grade 4 (see Paz-Ares, L. G. et al. (2011) Journal of Clinical Oncology 29, 3783-3790). This study showed a clinical benefit rate (ratio of patients having a stable disease, partial response or complete response) of about 50% with doses between 50 and 1,400 mg antibody. About 5% of the patients had a partial or complete response, and of the patients treated with less than 400 mg antibody, only 18% showed a stable disease. Furthermore, shrinkage in tumor size was observed for 26% of the patients, each of which were treated with 40 mg antibody or more. The average duration of progression-free survival was 121 days. However, such clinical benefit rates were often only achieved with patients that received their first regular chemotherapeutic treatment. In the intend to treat population, the clinical benefit rate was lower. Additionally, due to the high number of adverse skin reactions, 15% of the patients required a dose delay and 8% required a dose reduction. Patients also withdrew from the study because of adverse skin reactions. 32% of the patients had to be treated additionally at the end of the study for ongoing rash. Skin rashes were also observed at high incidences when the antibody was given at a low dosage. Therefore, also with these novel, optimized anti-EGFR antibodies adverse skin reactions occurred, including severe forms. It was again reported that the incidence of rash was consistent with that previously reported for cetuximab and panitumumab where it was considered to correlate with activity. Other adverse events common for anti-EGFR antibodies that also occurred with GA201 were hypomagnesemia and infusion related reactions (77%).

Similar results were also obtained for the human monoclonal anti-EGFR antibody zalutumumab. Treatment of cancer patient with metastatic squamous cell carcinoma of the head and neck showed a small to moderate increase in the overall survival and the progression-free survival. However, for 92% of the patients a skin rash was reported, with 21% of the patients having a grade 3 or 4 rash (see Machiels, J.-P. et al. (2011) Lancet Oncology 12, 333-343).

However, these commonly occurring skin reactions are highly problematic for EGFR inhibitor treatments for several different aspects. For one, cancer patients who are treated with the EGFR inhibitor are commonly already in a rather poor general health condition and the adverse skin reactions further deteriorate the patient's status which thereby becomes even more critical. Therefore, these patients can hardly tolerate the further burden of the adverse skin reactions caused by conventional EGFR inhibitor therapy or the burden of additional medication to treat the skin reactions. Furthermore, if the skin reactions develop into a severe form, they may be accompanied by skin necrosis or secondary infections which may readily become troublesome or even life-threatening for cancer patients. Subsequent to the development of severe dermatologic toxicities, complications including S. aureus sepsis and abscesses requiring incision and drainage were reported. Furthermore, in July 2012, Amgen has sent out a red-hand letter wherein the medical practitioner was informed and warned that during the treatment with the anti-EGFR antibody panitumumab life-threatening and infectious complications with death incidences occurred during treatment that were associated with severe adverse skin reactions caused by the treatment, in particular necrotizing fasciitis and sepsis. Especially late stage cancer patients often have only a reduced immune response after different chemotherapeutic treatments and any further complication should be avoided. But also for patients with a better general health status the adverse skin reactions may become a serious complication also due to their tremendous negative effect on the psychological level. EGFR inhibitor induced skin rash often affects the upper body area including the face and hence, cannot be hidden. Therefore, they are visible to others. For many patients even less severe adverse skin reactions may develop into an unbearable psychological burden and can result in discontinuation of treatment.

Moreover, up to date there is no general means for treatment or prevention of these adverse skin reactions (see Li, T. and Perez-Soler, R. (2009) Targeted Oncology 4, 107-119, and Perez-Soler, R. et al. (2005) The Oncologist 10, 345-356). Rather, each patient has to be treated individually for the skin reactions in addition to the cancer treatment with the EGFR inhibitor. In the prescribing information for Erbitux® a prophylactic co-treatment with oral tetracyclines and other therapies is recommended. However, the literature describes that new therapies for managing adverse skin reactions occurring during EGFR inhibitor treatment, in particular anti-EGFR antibody treatment are urgently needed.

The materiality and impact of adverse skin reaction, in particular of severe forms such as grade 3 or higher, on the treatment also becomes apparent from the fact that the treatment with an anti-EGFR antibody needs to be adapted if severe adverse skin reactions occur. E.g. if severe adverse skin reactions of grade 3 or higher occur during the treatment with cetuximab, the treatment must be interrupted and may only be continued if the adverse skin reaction is reduced to grade 2. If adverse skin reactions of grade 3 or higher reoccur for a second or third time, the treatment must be interrupted and the dosage must be reduced when continuing the treatment. If the adverse skin reaction again reoccurs or the adverse skin reaction does not regress to a grade 2, treatment with cetuximab must even be terminated (see Erbitux® prescribing information). Similar treatment regimens are provided for the anti-EGFR inhibitor panitumumab (see prescribing information). Therefore, the only way to handle severe adverse skin reactions is a dose reduction or an interruption or termination of the EGFR inhibitor treatment (see, also Fracasso, P. M. et al. (2007) Clinical Cancer Research 13, 986-993 and Li, T. and Perez-Soler, R. (2009)). However, in case of a treatment interruption or termination the cancer is no longer treated and further tumor progression and metastatic spread may thus well be a direct result of the adverse skin reactions in particular in patients with no other treatment options. In patients with advanced cancer, this may have dramatic consequences. Similar problems may occur during a dose reduction as the efficacy can be reduced.

Therefore, evidently these skin reactions are material and relevant for the treatment with anti-EGFR antibodies and other EGFR inhibitors and also have a severe impact on the treatment schedule and patient management. Therefore, there is a need for novel treatments with anti-EGFR antibodies that avoid or reduce the occurrence of adverse skin reactions, in particular severe skin reactions of grade 3 and higher.

Another major complication with cancer patients are malignant effusions (also called malignancy-related effusions, i.e. the escape of fluid e.g. from the blood or lymph vessels into body cavities caused by a malignant cancer disease) (see, e.g., Covey, A. M. (2005) The Journal of Supportive Oncology 3, 169-176 and Olopade, O. I. and Ultmann, J. E. (1991) Cancer Journal for Clinicians 41, 166-179). These effusions commonly have to be drained by punctuation to give the patients at least some relieve. However, this procedure is very painful and poses the risk of complications such as secondary infections or hypotension. Furthermore, effusion drainage only targets the symptoms and has to be repeated frequently which increases the pain and complication risk and is very cumbersome for the patient. Effusions are caused by a deregulation of the fluid transport through the membranes lining the body cavity.

Primary tumors or metastases affecting the membrane (mesothelium) lining the body cavity may, for example, result in uncontrolled fluid input and/or a disturbed fluid output. In particular, pleural effusions and peritoneal effusions (i.e. ascites) affect many patients with advanced or terminal cancer. Pleural effusion is an excessive fluid collection in the pleural cavity and is often associated with dyspnea, cough and chest pain. The most common types of cancer which underlie malignant pleural effusions are lung and breast cancer as well as lymphomas. However, in principle any cancer can result in malignant effusion due to metastatic spread which may also affect the respective mesothelium. Malignant ascites is mainly associated with ovarian, breast, gastric, pancreatic, hepatic and colon cancer and lymphoma. Besides the pain and burden directly caused by the effusion, it also greatly adversely affects the quality of life of the patients due to a resulting immobility, the effort for drainage of the effusion and the pain resulting therefrom. However, since malignant effusions often develop in patients with advanced or even terminal cancer, standard cancer therapies in many cases do not have an effect on the effusion. The importance of effusions in the context of cancer treatment is also highlighted by drugs which are specifically approved for therapy of malignant effusions. Hence, malignant effusions are seen as separate, additional indication which has to be treated. For example, catumaxomab (Removab® by Trion Pharma) is a bispecific antibody used for the treatment of malignant ascites. However, this leads to a further therapy—in addition to the cancer treatment—which is commonly associated with further adverse side effects and significantly adds to the burden for the cancer patient. For example, catumaxomab causes adverse reactions in about 90% of the treated patients (see prescribing information for Removab of Fresenius Biotech GmbH).

Therefore, there is also a need for novel treatments of malignant effusions. In particular, there is a need for an anticancer treatment which also allows to treat malignant effusions.

In view of the above, it is evident that there is a great need for novel and improved treatments of EGFR positive neoplastic diseases, in particular EGFR positive malignant epithelial tumors. In particular, there is a high demand to provide effective treatments of EGFR positive cancer with anti-EGFR antibodies which, however, address the problems associated with the highly common severe adverse skin reactions caused by conventional anti-EGFR antibody therapy. Furthermore, novel treatments are required, which are also effective against kidney cancer, in particular clear cell and non-clear cell renal carcinomas. In addition, there is also a great need for cancer treatments which effectively prevent or reduce malignant effusions.

### SUMMARY OF THE INVENTION

The anti-EGFR antibodies according to the present invention having a reduced (including absent) fucosylation in their Fc region demonstrate in clinical trials a remarkable therapeutic profile and high therapeutic efficacy. E.g. said anti-EGFR antibodies are effective against a wide range of different EGFR-positive neoplastic diseases, such as colon cancer, lung cancer, gastric cancer, and kidney cancer including different types of renal cell carcinomas. Said anti-EGFR antibodies were also found to be effective against different forms of metastases. Furthermore, treatment with the anti-EGFR antibodies according to the present invention is also highly effective against effusions associated with EGFR positive malignant diseases, and is in particular effective in treating pleural and/or peritoneal effusions. Hence, the present invention provides novel treatments for patients suffering from said diseases, even in a monotherapy setting.

At the same time, the present inventors surprisingly found that the anti-EGFR antibodies according to the present invention cause significantly less and milder adverse skin reactions than conventional EGFR inhibitors, in particular than presently used anti-EGFR antibodies such as cetuximab (Erbitux®, Merck), panitumumab (Vectibix®, Amgen) or newly developed anti-EGFR antibodies such as GA201. As described above, conventional anti-EGFR antibodies are always associated with severe adverse reactions, especially adverse skin reactions such as acneiform rash. Because these adverse skin reactions are so common for anti-EGFR antibodies, they were in the prior art even being considered as indicator for the therapeutic efficacy of the EGFR inhibitors (see above). The present invention, however, disproves the assumption that an efficient treatment with an EGFR inhibitor is inevitably associated with adverse skin reactions, including severe skin reactions in a large amount of the treated patients. As is shown by the clinical data presented herein, the anti-EGFR antibodies according to the present invention show only mild to moderate adverse skin reactions and even these mild skin reactions occur only in less than 50% of the treated patients. No severe adverse skin reaction of grade 3 or higher was observed so far in clinical studies. This is a highly remarkable property which distinguishes the reduced fucose anti-EGFR antibodies of the invention from prior art anti-EGFR antibodies such as Erbitux®, panitumumab, zalutumumab and GA201, which cause adverse skin reactions in 80% or more of the patients and in many cases cause adverse skin reactions of grade 3 and higher. Nevertheless, a high therapeutic efficacy is demonstrated for the reduced fucose anti-EGFR antibody according to the present invention in in vitro tests, including fully human assays, and clinical studies. Furthermore, as is shown by the clinical data provided herein, also the general side effect profile is improved in the reduced fucose anti-EGFR antibody of the invention what is important for long term therapy. For example, the anti-EGFR antibodies according to the present invention have a reduced risk of hypomagnesemia, a health-critical low blood magnesium level, while the treatment with conventional anti-EGFR antibodies results in a progressive magnesium loss in almost all treated patients, leading to hypomagnesemia in up to 55% of the patients. Furthermore, no allergic reactions were reported and the incidence of other adverse reactions such as hypokalemia or diarrhoe was significantly reduced compared to standard therapy. Therefore, the treatment with the anti-EGFR antibody according to the present invention is better tolerated by the patients. This is an important advantage considering the health condition of cancer patients, in particular heavily pretreated patients.

As will be described in detail in the following, the high therapeutic efficacy combined with the significant lower degree of skin toxicity and overall improved side effect profile that is achieved with the anti-EGFR antibody of the invention provides important new treatment options and in particular allows the effective treatment of novel patient groups that cannot be treated with common anti-EGFR antibodies which are known to cause severe adverse skin reactions of grade 3 or higher. In particular, patients who cannot or cannot further be treated with conventional anti-EGFR antibodies, e.g. because the health risk due to these adverse reactions outweigh the potential health benefit received by the treatment, and patients whose treatment with a conventional anti-EGFR antibody had to be interrupted or terminated because adverse reactions, in particular severe adverse skin reactions, occurred, now can be treated with the reduced fucose EGFR antibodies according to the present invention. Thereby, novel and important new treatment options are provided which is an important contribution to the prior art.

Based on the above findings, the present invention provides an anti-EGFR antibody with a glycosylation site in the CH2 domain, wherein 50% or less, 40% or less, 30% or less, preferably 25% or less, more preferably 20% or less, more preferably 15% to 0% of the glycans attached to said glycosylation site carry fucose (reduced fucose anti-EGFR antibody) and wherein the reduced fucose anti-EGFR antibody is capable of inducing an antibody-dependent cellular cytotoxicity reaction, for treating a human patient with an EGFR positive neoplastic disease, in particular cancer. As described above, the reduced fucose anti-EGFR antibody of the invention causes less adverse skin reactions than prior art anti-EGFR antibodies. In certain embodiments, the reduced fucose anti-EGFR antibody of the invention causes adverse skin reactions of grade 3 or higher in not more than 10%, preferably not more than 3%, more preferred not more than 1% of the treated patients.

The anti-EGFR antibodies of the invention provide numerous novel treatment options. Inter alia, they can be used as first line treatment of an EGFR positive neoplastic disease and/or as follow on treatment of patients which previously received one or more anti-cancer treatments. Different treatments of EGFR positive neoplastic diseases are described in detail in the detailed description of the invention. Some aspects for treating EGFR positive neoplastic diseases are highlighted in the following.

According to one aspect, the reduced fucose anti-EGFR antibody of the invention is for treating a human patient who has been previously treated with at least one EGFR inhibitor, in particular an anti-EGFR antibody such as cetuximab or panitumumab. According to certain embodiments, this previous treatment caused an adverse skin reaction of grade 3 or higher in said patient.

According to a further aspect, the reduced fucose anti-EGFR antibody of the invention is for treating a human patient with known severe adverse skin reaction of grade 3 or 4 against an anti-EGFR antibody which causes such severe adverse skin reactions, in particular cetuximab (Erbitux®) or panitumumab.

According to a further aspect, the reduced fucose anti-EGFR antibody of the invention is for treating a human patient at risk of developing a severe adverse skin reaction of grade 3 or 4 during treatment with an anti-EGFR antibody which causes severe adverse skin reactions of grade 3 or higher in more than 12% of the treated patients.

According to a further aspect, the reduced fucose anti-EGFR antibody of the invention is for treating a human patient who has been previously treated with an EGFR inhibitor, in particular an anti-EGFR antibody, and wherein said previous treatment was interrupted, terminated or wherein the dosage of the EGFR inhibitor had to be reduced because an adverse skin reaction occurred during said previous treatment.

According to a further aspect, the reduced fucose anti-EGFR antibody of the invention is for treating a human patient who has been previously treated with an EGFR inhibitor, in particular an anti-EGFR antibody, and wherein said previous treatment with the EGFR inhibitor is not or cannot be continued because an adverse skin reaction against said EGFR inhibitor occurred.

According to a further aspect, the reduced fucose anti-EGFR antibody according to the present invention is used for treating an EGFR positive neoplastic disease in a human patient under conditions which, for at least one other anti-EGFR antibody, cause an adverse skin reaction in at least 50% of the patients when treating patients with said other anti-EGFR antibody under said conditions, or under conditions which, for at least one other anti-EGFR antibody, cause an adverse skin reaction of grade 3 or higher in at least 12% of the patients when treating patients with said other anti-EGFR antibody under said conditions. The at least one other anti-EGFR antibody in particular is selected from the group consisting of high fucose cetuximab (Erbitux®), panitumumab, zalutumumab and GA201. In this respect, “under conditions” in particular refers to the dosage of the anti-EGFR antibody.

According to one aspect, the reduced fucose anti-EGFR antibody of the invention is for treating a human patient that is afflicted with an EGFR positive neoplastic disease for which disease it has been shown that at least one other anti-EGFR antibody shows adverse skin reactions in more than 50%, more than 60% or more than 70% of the treated patients. Examples of such EGFR positive neoplastic diseases and preferred embodiments are described herein and it is referred to the respective disclosure. In certain embodiments, the reduced fucose antibody of the invention is administered at a dosage wherein the other anti-EGFR antibody shows adverse skin reactions in more than 50%, more than 55%, more than 60%, more than 65%, more than 70% or more than 75% of the patients. In certain embodiments, the other anti-EGFR antibody is selected from high fucose Cetuximab (Erbitux®), panitumumab, zalutumumab and GA201. In one embodiment, the other anti-EGFR antibody is cetuximab (Erbitux®). In certain embodiments, the reduced fucose antibody is administered at an average dosage of at least 200 mg per week, preferably 240 mg per week, 300 mg per week, 400 mg per week or 500 mg per week, preferably in one dose every week (with the indicated amount per dose) or every second week (with the double amount per dose). In certain embodiments, the adverse skin reactions caused by the other anti-EGFR antibody include adverse skin reactions of grade 3 or higher. In certain embodiments, the adverse skin reaction caused by the other anti-EGFR antibody, which according to one embodiment is Cetuximab (Erbitux®), is or includes acneiform skin rash. The reduced fucose antibody of the invention may be administered at a dosage wherein the other anti-EGFR antibody shows adverse skin reactions of grade 3 or higher in more than 12%, more than 15%, or more than 17% of the patients. As described herein, the reduced fucose anti-EGFR antibody of the invention has the advantage that the incidence of adverse skin reactions, in particular adverse skin reactions of grade 3 or 4, is significantly reduced or can even be prevented. Therefore, the present invention allows to reduce or prevent side effects, in particular adverse skin reactions, during treatment of an EGFR positive neoplastic disease with an anti-EGFR antibody, by administering the reduced fucose anti-EGFR antibody of the invention. In particular, adverse skin reactions of grade 3 or 4 can be significantly reduced or prevented by the present invention. This advantageous effect is achieved even if average dosages of at least 200 mg per week, preferably 240 mg per week, 300 mg per week, 400 mg per week or 500 mg per week of the reduced fucose anti-EGFR antibody are administered to the patient and even when treating EGFR positive neoplastic diseases such as colorectal cancer which have a particular high incidence of adverse skin reactions such as skin rash when treated with another anti-EGFR antibody such as cetuximab (Erbitux®). Prior art anti-EGFR antibodies such as cetuximab (Erbitux®) cause when used at such dosages adverse skin reactions in more than 75% of the patients and also cause adverse skin reactions of grade 3 or 4. Therefore, the reduced fucose anti-EGFR antibody according to the invention provides novel treatment options. The reduced fucose antibody of the invention can be used as first line, second line or later treatment. In certain embodiments, the patient that is treated with the reduced fucose anti-EGFR antibody of the invention has been previously treated with an anti-EGFR antibody which causes adverse skin reactions in more than 50%, more than 60% or more than 70% of the treated patients. In certain embodiments, the previous treatment with the anti-EGFR antibody had to be terminated, interrupted or the dosage had to be reduced because adverse skin reactions occurred, in particular adverse skin reactions of grade 3 or 4 and/or because treatment failed with the previously used anti-EGFR antibody. Details of such a pretreatment as well as suitable and preferred embodiments are also described in the detailed description of the invention and is referred to said disclosure. Furthermore, therein also specific embodiments of the EGFR positive neoplastic disease, suitable and preferred dosages and patient groups are described. The respective disclosure can be combined with the aspect of the invention described in this paragraph. In certain embodiments, the reduced fucose anti-EGFR antibody of the invention is for long term treatment of at least 3 months, at least 6 months, at least 9 months or at least 12 months.

According to a further aspect, the reduced fucose anti-EGFR antibody of the invention is for treating a human patient who has developed an allergic reaction against a therapeutic antibody in a previous treatment. In particular, the patient may have developed an allergic reaction against a therapeutic antibody produced in a rodent cell. In certain embodiments, the patient has developed an allergic reaction against an anti-EGFR antibody in a previous treatment.

According to a further aspect, the reduced fucose anti-EGFR antibody of the invention is for treating a human patient with pre-existing Gal-Gal IgE antibodies.

According to a further aspect, the reduced fucose anti-EGFR antibody of the invention is for treating a malignant effusion, in particular a malignant pleural or peritoneal effusion, in particular malignant ascites in a human patient having an EGFR positive neoplastic disease, in particular cancer.

According to a further aspect, the reduced fucose anti-EGFR antibody of the invention is for treating EGFR positive renal cell carcinoma, in particular for treating a clear cell renal cell carcinoma or for treating a non-clear cell renal cell carcinoma.

Furthermore, in a further aspect the present invention provides a method of treatment of an EGFR positive neoplastic disease in a human patient, comprising administering to the patient a therapeutically effective amount of the reduced fucose anti-EGFR antibody according to the invention, wherein the reduced fucose anti-EGFR antibody is capable of inducing an antibody-dependent cellular cytotoxicity reaction.

In another aspect, the present invention provides a method for reducing the adverse reactions in a treatment of a patient having an EGFR positive neoplastic disease with an EGFR inhibitor, comprising the step of treating the patient with the reduced fucose anti-EGFR antibody according to the invention. In particular, the method includes terminating the treatment with the EGFR inhibitor and starting a treatment with the reduced fucose anti-EGFR antibody according to the invention.

All the embodiments and features described herein for the reduced fucose anti-EGFR antibody according to the invention also likewise apply to the methods of treatment and other methods according to the invention. In particular, the uses of the reduced fucose anti-EGFR antibody according to the invention for treating a patient also refer to a respective method of treating said patient.

The above aspects can be combined. Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### DEFINITIONS

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements may and/or indeed does encompass further elements as well as embodiments wherein the subject-matter which “comprises” specifically listed elements does not comprise further elements. Likewise, the expression “have” is to be understood as the expression “comprise”, also including and specifically referring to the expressions “consist essentially of” and “consist of”.

The term “antibody” in particular refers to a protein comprising at least two heavy chains and two light chains connected by disulfide bonds. Each heavy chain is comprised of a heavy chain variable region (VH) and a heavy chain constant region (CH). Each light chain is comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The heavy chain constant regions may be of any type such as γ-, δ-, α-, μ- or ε-type heavy chains. Preferably, the heavy chain of the antibody is a γ-chain. Furthermore, the light chain constant region may also be of any type such as κ- or λ-type light chains. Preferably, the light chain of the antibody is a κ-chain. The constant regions of the antibodies may mediate the binding of the immunoglobulin to host tissues or factors, including various cells of the immune system (e.g., effector cells) and the first component (C1q) of the classical complement system. The antibody can be e.g. a humanized, human or chimeric antibody. The antibody according to the invention is capable of inducing ADCC.

The antigen-binding portion of an antibody usually refers to full length or one or more fragments of an antibody that retain the ability to specifically bind to an antigen. It has been shown that the antigen-binding function of an antibody can be performed by fragments of a full-length antibody. Examples of binding fragments of an antibody include a Fab fragment, a monovalent fragment consisting of the V_{L}, V_{H}, C_{L} and CH1 domains; a F(ab)₂ fragment, a bivalent fragment comprising two Fab fragments, each of which binds to the same antigen, linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR). The “Fab part” of an antibody in particular refers to a part of the antibody comprising the heavy and light chain variable regions (VH and VL) and the first heavy and light chain constant regions (CH1 and CL). In cases where the antibody does not comprise all of these regions, then the term “Fab part” only refers to those of the regions VH, VL, CH1 and CL which are present in the antibody. Preferably, “Fab part” refers to that part of an antibody corresponding to the fragment obtained by digesting a natural antibody with papain which contains the antigen binding activity of the antibody. In particular, the Fab part of an antibody encompasses the antigen binding site or antigen binding ability thereof. Preferably, the Fab part comprises at least the VH region of the antibody.

The “Fc part” of an antibody in particular refers to a part of the antibody comprising the heavy chain constant regions 2, 3 and—where applicable—4 (CH2, CH3 and CH4). In cases where the antibody does not comprise all of these regions, then the term “Fc part” only refers to those of the regions CH2, CH3 and CH4 which are present in the antibody. Preferably, the Fc part comprises at least the CH2 region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises a Fc receptor binding site or a Fc receptor binding ability.

For indicating the amino acid positions of the heavy chain and light chain, in particular the variable regions thereof, the Kabat numbering system is used herein (Kabat, E. A. et al. (1991) Sequences of Proteins of Immunological Interest, 5^{th} edition, NIH Publication No. 91-3242). According to said system, the heavy chain variable region comprises amino acid positions from position 0 to position 113 including position 35A, 35B, 52A to 52C, 82A to 82C and 100A to 100K. The CDRs of the heavy chain variable region are located, according to the Kabat numbering, at positions 31 to 35B (CDR1), 50 to 65 (CDR2) and 95 to 102 (CDR3). The remaining amino acid positions form the framework regions FR1 to FR4. The light chain variable region comprises positions 0 to 109 including positions 27A to 27F, 95A to 95F and 106A. The CDRs are located at positions 24 to 34 (CDR1), 50 to 56 (CDR2) and 89 to 97 (CDR3). Depending on the initial formation of the specific gene of an antibody, not all of these positions have to be present in a given heavy chain variable region or light chain variable region. In case an amino acid position in a heavy chain or light chain variable region is mentioned herein, unless otherwise indicated it is referred to the position according to the Kabat numbering.

According to the present invention, the term “chimeric antibody” in particular refers to an antibody wherein the constant regions are derived from a human antibody or a human antibody consensus sequence, and wherein at least one and preferably both variable regions are derived from a non-human antibody, e.g. from a rodent antibody such as a mouse antibody.

According to the present invention, the term “humanized antibody” in particular refers to a non-human antibody comprising human constant regions and variable regions which amino acid sequences are modified so as to reduce the immunogenicity of the antibody when administered to the human body. An exemplary method for constructing humanized antibodies is CDR grafting, wherein the CDRs or the specificity determining residues (SDRs) of a non-human antibody are combined with human-derived framework regions. Optionally, some residues of the human framework regions may be backmutated towards the residues of the parent non-human antibody, e.g. for increasing or restoring the antigen binding affinity. Other humanization methods include, for example, resurfacing, superhumanization, and human string content optimization. In the resurfacing methods, only those residues of the non-human framework regions which are positioned at the surface of the antibody are replaced by residues present in corresponding human antibody sequences at said position. Superhumanization essentially corresponds to CDR grafting. However, while during CDR grafting the human framework regions are normally chosen based on their homology to the non-human framework regions, in superhumanization it is the similarity of the CDRs on the basis of which the human framework regions are chosen. In the human string content optimization the differences of the non-human antibody sequence to the human germline sequences is scored and then the antibody is mutated to minimize said score. Furthermore, humanized antibodies can also be obtained by empirical methods wherein large libraries of human framework regions or human antibodies are used to generate multiple antibody humanized candidates and then the most promising candidate is determined by screening methods. Also with the above-described rational approaches several humanized antibody candidates can be generated and then screened, for example for their antigen binding. Overviews of humanization processes can be found, for example, in Almagro, J. C. and Fransson, J. (2008) Frontiers in Bioscience 13, 1619-1633 and in the entire volume 36 of the Journal Methods (2005).

The term “human antibody”, as used herein, is intended to include antibodies having variable regions in which both the framework and CDR regions are derived from sequences of human origin.

Furthermore, the antibody according to the present invention may have been subjected to framework or Fc engineering. Such engineered antibodies include those in which modifications have been made to framework residues within V_{H} and/or V_{L}, e.g. to improve the properties of the antibody. Typically such framework modifications are made to decrease the immunogenicity of the antibody. For example, during humanization, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies can also be used according to the present invention. In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. E.g., the Fc region can be altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. In one embodiment, the Fc region of the described antibodies is modified to increase the ability of the antibody to mediate antibody dependent cellular cytotoxicity (ADCC) and/or to increase the affinity of the antibody for an Fcγ receptor by modifying one or more amino acids. This approach is described further e.g. in WO00/42072. Moreover, the binding sites on human IgG1 for FcγRI, FcγRII, FcγRIII and FcRn have been mapped and variants with improved binding have been described (see Shields, R. L. et al., 2001 J. Biol. Chen. 276:6591-6604).

A target amino acid sequence is “derived” from or “corresponds” to a reference amino acid sequence if the target amino acid sequence shares a homology or identity over its entire length with a corresponding part of the reference amino acid sequence of at least 75%, more preferably at least 80%, at least 85%, at least 90%, at least 93%, at least 95% or at least 97%. For example, if a framework region of a humanized antibody is derived from or corresponds to a variable region of a particular human antibody, then the amino acid of the framework region of the humanized antibody shares a homology or identity over its entire length with the corresponding framework region of the human antibody of at least 75%, more preferably at least 80%, at least 85%, at least 90%, at least 93%, at least 95% or at least 97%. The “corresponding part” means that, for example, framework region 1 of a heavy chain variable region (FRH1) of a target antibody corresponds to framework region 1 of the heavy chain variable region of the reference antibody. In particular embodiments, a target amino acid sequence which is “derived” from or “corresponds” to a reference amino acid sequence is 100% homologous, or in particular 100% identical, over its entire length with a corresponding part of the reference amino acid sequence. A “homology” or “identity” of an amino acid sequence or nucleotide sequence is preferably determined according to the invention over the entire length of the reference sequence or over the entire length of the corresponding part of the reference sequence which corresponds to the sequence which homology or identity is defined.

“Specific binding” preferably means that an agent such as an antibody binds stronger to a target such as an epitope for which it is specific compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 100-fold, 200-fold, 500-fold or more than 1000-fold lower than the dissociation constant for the target to which the agent does not bind specifically. Furthermore, the term “specific binding” in particular indicates a binding affinity between the binding partners with a Kₐ of at least 10⁶ M⁻¹, preferably at least 10⁷ M⁻¹, more preferably at least 10⁸ M⁻¹. An antibody specific for a certain antigen in particular refers to an antibody which is capable of binding to said antigen with an affinity having a Kₐ of at least 10⁶ M⁻¹, preferably at least 10⁷ M⁻¹, more preferably at least 10⁸ M⁻¹. For example, the term “anti-EGFR antibody” refers to an antibody specifically binding EGFR and preferably is capable of binding to EGFR with an affinity having a Kₐ of at least 10⁶ M⁻¹, preferably at least 10⁷ M⁻¹, more preferably at least 10⁸ M⁻¹.

The term “cetuximab” as used herein in particular refers to the antibody cetuximab having the amino acid sequences of the cetuximab antibody as used in the medicament Erbitux® (Merck). As long as the circumstances do not indicate otherwise, the antibody cetuximab also has in its Fc part the same or a similar high fucose glycosylation pattern as the cetuximab antibody used in the medicament Erbitux® (Merck), wherein the fucosylation is at least 60%, in particular at least 70%. Circumstances that indicate a different glycosylation pattern are, for example, the reference to “Fuc− cetuximab”. The term “Fuc− cetuximab” in particular refers to an antibody binding the same epitope as cetuximab and having amino acid sequences which are at least 85%, preferably at least 90%, more preferred at least 95% identical to those of the cetuximab antibody as used in the medicament Erbitux® (Merck), wherein, however, the Fuc− cetuximab has a lower amount of fucose in its Fc part than the cetuximab antibody used in the medicament Erbitux® and in particular has a fucosylation in the Fc part of 50% or less, 30% or less, preferably 25% or less, more preferred 20% or less and most preferred 15% to 0%.

The term “EGFR” according to the present invention in particular refers to the human epidermal growth factor receptor 1, also known as ErbB-1 or HER1. EGFR is a receptor tyrosine kinase comprising an extracellular ligand binding domain, a membrane-spanning domain and an intracellular kinase domain. Upon binding of its ligand (e.g. epidermal growth factor (EGF) and transforming growth factor α (TGFα)), the EGFR forms homodimers or heterodimers with other ErbB receptors and its kinase function is activated, resulting in the autophosphorylation of several tyrosines of the intracellular domain. An anti-EGFR antibody is an antibody which is capable of specifically binding EGFR. In certain embodiments, an anti-EGFR antibody is capable of interfering with or inhibiting activation of EGFR, e.g. by preventing ligand binding to and/or dimerization of the receptor. In certain embodiments, an anti-EGFR antibody is capable of inhibiting the proliferation of EGFR positive human cancer cells. In certain embodiments, the anti-EGFR antibody is capable of specifically binding to EGFR, but does not prevent or reduce EGFR signaling. In this case, the anti-EGFR antibody preferably is therapeutically active via the ADCC mechanism.

The term “antibody”, as used herein, refers in certain embodiments to a population of antibodies of the same kind. In particular, all antibodies of the population of the antibody exhibit the features used for defining the antibody. In certain embodiments, all antibodies in the population of the antibody have the same amino acid sequence. Reference to a specific kind of antibody, such as an anti-EGFR antibody or a reduced fucose anti-EGFR antibody, in particular refers to a population of this kind of antibody.

Preferably, all antibodies in a population of the reduced fucose anti-EGFR antibody have the same amino acid sequence. The population of the reduced fucose anti-EGFR antibody may be present in a composition which also comprises other antibodies. These other antibodies are not included in the term “reduced fucose anti-EGFR antibody” and are not considered for determining the glycosylation characteristics of the reduced fucose anti-EGFR antibody. According to the invention, the (percentage) amount of fucose in the Fc part and thus the CH2 domain of a reduced fucose anti-EGFR antibody in particular refers to the percentage of all carbohydrate chains attached to the corresponding glycosylation site in the CH2 domain of the reduced fucose anti-EGFR antibodies in the population of the reduced fucose anti-EGFR antibody which comprise a fucose residue. Said carbohydrate chains include the carbohydrate chains attached to the glycosylation site corresponding structurally or by amino acid sequence homology to amino acid position 297 according to the Kabat numbering of the heavy chain of IgG-type antibodies. The N-linked glycosylation at Asn297 is conserved in mammalian IgGs as well as in homologous regions of other antibody isotypes. Antibodies usually comprise two heavy chains and two light chains and hence, have two glycosylation sites in their Fc part, one in each CH2 domain. For the avoidance of doubt, it is provided that it is not mandatory that both glycosylation sites in the CH2 domains of the antibody have to carry a carbohydrate chain. It is not distinguished between the two glycosylation sites in the two CH2 domains and referring to a glycosylation domain in the CH2 domain also refers to both glycosylation sites in both CH2 domains. Preferably, only fucose residues are considered which are bound via an α1,6-linkage to the GlcNAc residue at the reducing end of the carbohydrate chain. If the amount of fucose in the CH2 domain of a specific antibody species (e.g. reduced fucose anti-EGFR antibodies) is mentioned, then only the carbohydrate chains attached to the glycosylation site of the CH2 domains of the antibody molecules of the population of said specific antibody species (e.g. the population of the reduced fucose anti-EGFR antibody) in a composition are considered for determining the percentage amount of fucose, i.e. the amount of carbohydrate chains carrying a fucose.

Carbohydrate chains attached to glycosylation sites in the Fab part of the antibody, if present, as well as carbohydrate chains attached to other antibodies, if present in a composition together with the reduced fucose anti-EGFR antibody, are not considered for determining the amount of fucose in the CH2 domain of the reduced fucose anti-EGFR antibody. Carbohydrates attached to the Fab part and the Fc part of an antibody can be determined separately by first digesting the antibody in a Fab part and a Fc part, separating the parts from each other and individually determining the glycosylation features of each part. Likewise, the (percentage) amount of bisecting N-acetylglucosamine (bisGlcNAc) attached to the CH2 domain of the reduced fucose anti-EGFR antibody in particular refers to the percentage of all carbohydrate chains attached to the glycosylation site in the CH2 domain of all antibodies in the population of the reduced fucose anti-EGFR antibody which comprise a bisGlcNAc residue. BisGlcNAc refers to a GlcNAc residue attached to the central mannose residue in complex type N-glycans. Likewise, the (percentage) amount of galactose attached to the CH2 domain of the reduced fucose anti-EGFR antibody in particular refers to the percentage of all carbohydrate chains attached to the glycosylation site in the CH2 domain of all antibodies in the population of the reduced fucose anti-EGFR antibody which comprise at least one galactose residue. The above considerations which are described for reduced fucose anti-EGFR antibodies likewise apply to other specific antibodies such as high fucose anti-EGFR antibodies or the like.

An anti-EGFR antibody having in a specific domain, such as the CH2 domain or the VH domain, a relative amount of glycans carrying a specific saccharide unit or feature, such as fucose, galactose, two galactoses, bisecting GlcNAc, sialic acid or two sialic acids, of a specific percentage value or range in particular refers to a population of anti-EGFR antibodies all having the same amino acid sequence, wherein said percentage or percentage range of all glycans attached to said specific domain of all anti-EGFR antibodies of the population comprise said specific saccharide unit or fulfill the feature. Likewise, an anti-EGFR antibody having an amount of a specific saccharide unit or feature in the glycans attached to a specific domain which is a specific percentage value or range also in particular refers to a population of anti-EGFR antibodies all having the same amino acid sequence, wherein said percentage or percentage range of all glycans attached to said specific domain of all anti-EGFR antibodies of the population comprise said specific saccharide unit or fulfill the feature. The terms “carbohydrate chain”, “carbohydrate structure”, “glycan” and “glycan structure” as used herein have the same meaning and are used interchangeably.

According to the invention, the term “glycosylation site” in particular refers to an amino acid sequence which can specifically be recognized and glycosylated by a natural glycosylation enzyme, in particular a glycosyltransferase, preferably a naturally occurring mammalian or human glycosyltransferase. In particular, the term “glycosylation site” refers to an N-glycosylation site, comprising an asparagine residue to which the carbohydrate is or can be bound. In particular, the glycosylation site is an N-glycosylation site which has the amino acid sequence Asn-Xaa-Ser/Thr/Cys, wherein Xaa is any amino acid residue. Preferably, Xaa is not Pro.

In a “conjugate” two or more compounds are linked together. In certain embodiments, at least some of the properties from each compound are retained in the conjugate. Linking may be achieved by a covalent or non-covalent bond. Preferably, the compounds of the conjugate are linked via a covalent bond. The different compounds of a conjugate may be directly bound to each other via one or more covalent bonds between atoms of the compounds. Alternatively, the compounds may be bound to each other via a chemical moiety such as a linker molecule wherein the linker is covalently attached to atoms of the compounds. If the conjugate is composed of more than two compounds, then these compounds may, for example, be linked in a chain conformation, one compound attached to the next compound, or several compounds each may be attached to one central compound.

The term “patient” in particular refers to a human being.

The terms “EGFR positive neoplastic disease” and “EGFR positive cancer” according to the invention which can be treated with the reduced fucose anti-EGFR antibody described herein in particular refer to a neoplastic disease, cancer, tumor and/or metastasis wherein cells express EGFR. EGFR positive cancers include but are not limited to malignant epithelial tumors, breast cancer, gastric cancer, cancer of the gastrointestinal tract, carcinomas, colon cancer, bladder cancer, urothelial tumors, uterine cancer, esophageal cancer, cancer of the gastroesophageal junction, ovarian cancer, lung cancer, endometrial cancer, kidney cancer, pancreatic cancer, thyroid cancer, colorectal cancer, prostate cancer, cancer of the brain, cervical cancer, intestinal cancer and liver cancer. In certain embodiments, the cancer is a metastasizing cancer. Preferably, the EGFR positive cancer to be treated with the reduced fucose anti-EGFR antibody is selected from colon cancer (including coecum and rectum cancer), kidney cancer, gastric cancer, head and neck cancer and lung cancer, esophageal cancer, endometrial and cervical cancer, in particular metastatic colon cancer, metastatic colorectal cancer, metastatic gastric cancer, (advanced) gastric adenocarcinomas, (advanced) esophageal adenocarcinomas, (advanced) gastroesophageal junction adenocarcinomas, (metastatic) renal cell carcinoma, (metastatic) non-small cell lung cancer (NSCLC), lung adenocarcinoma, squamous or non-squamous non-small cell lung cancer, triple negative breast cancer (breast cancer which is negative for the expression of estrogen receptor (ER), progesterone receptor (PR) and human epidermal growth factor receptor 2 (HER2)), squamous cell cancer of the head and neck, endometrical carcinoma or sarcoma, penis carcinoma, cervical carcinoma, malignant rhabdoid tumor and transitional cell carcinoma. Further EGFR positive cancers include renal cell carcinomas such as clear cell renal cell carcinoma, papillary renal cell carcinoma (basophilic and eosinophilic), chromophobe renal cell carcinoma, Bellini duct carcinoma/collecting duct carcinoma, and pleomorpohic (sarcomatoid) carcinoma of the kidney; non small cell lung cancers such as squamous non small cell lung cancer (sNSCLC), and non squamous non small cell lung cancer (nsNSCLC), in particular adenocarcinoma and large cell carcinoma; small cell lung cancer (SCLC); epithelial tumors of the head and neck such as squamous cell cancer of the head and neck (SCCHN), in particular non-differentiated, differentiated, adenoid-squamous and verrucous SCCHN; and gastric cancers such as adenocarcinoma, in particular tubular adenocarcinoma, papillary adenocarcinoma and mucinous adenocarcinoma, signet ring cell carcinoma, adenoid-squamous carcinoma, squamous carcinoma, medullary gastric carcinoma, small cell gastric carcinoma, and non-differentiated gastric carcinoma. The gastric cancer may be located in the pyloric antrum, in the corpus or in the fundus or may be a diffuse gastric cancer in the entire stomach.

In certain embodiments, the EGFR positive neoplastic disease or cancer which is treated with the reduced fucose anti-EGFR antibody of the invention comprises cancer cells which express EGFR. In certain embodiments, a tumor, metastasis or the like is classified as EGFR positive if a certain % of the comprised cells express EGFR. E.g. in the prior art a tumor is usually classified as being EGFR positive if at least 1% of the tumor cells express EGFR. The anti-EGFR antibody of the invention can be used for treating a human patient wherein prior to treatment the EGFR status was determined to be positive in said patient. Embodiments are described subsequently. However, the determination or confirmation of the EGFR status of the patient prior to initiating treatment with the anti-EGFR antibody of the invention is not necessary. E.g. there are diseases that are commonly known to be EGFR positive such as e.g. SCCHN. In case of SCCHN, more than 90% of the affected patients are EGFR positive. For diseases such as SCCHN or colorectal carcinoma which usually are EGFR positive, the EGFR status of the patient is often not determined prior to treatment with an anti-EGFR antibody in prior art approaches. Likewise, in such cases wherein a disease is to be treated wherein e.g. more than 75%, more than 80%, more than 85% or more than 90% of the patients are EGFR positive, treatment with the anti-EGFR antibody of the invention can be initiated without prior determination or confirmation of the EGFR status of the individual patient to be treated.

EGFR expression is detectable in gene or protein expression assays which detect the expression of the EGFR gene in cells of a cancer sample, e.g. by detecting the presence and/or amount of EGFR mRNA or EGFR protein. Suitable detection assays are, for example, immunohistochemistry (IHC), Western blot, enzyme-linked immunosorbent assay (ELISA), electro-chemiluminescence immunoassay (ECLIA), fluorescence-activated cell sorting (FACS), ligand-binding assay, polymerase chain reaction (PCR), Southern blot and in-situ hybridization such as fluorescence in-situ hybridization (FISH), silver in-situ hybridization (SISH) or chromogene in-situ hybridization (CISH), and other technologies known by those skilled in the art which can determine directly or indirectly the expression of the EGFR receptor on the surface of cells. Respective assays can be used for determining the EGFR status of the patient to be treated if such prior determination is desired.

According to certain embodiments, said EGFR positive neoplastic disease or cancer overexpresses EGFR and/or shows EGFR gene amplification. Accordingly, in certain embodiments the patient is afflicted with an EGFR positive cancer which comprises tumor cells and/or metastatic cells which express or even overexpress EGFR. EGFR expression can be determined e.g. by immunohistochemistry. Immunohistochemistry in this respect refers to the immunohistochemical staining of fixed tumor samples and the analysis of the staining. EGFR expression may be determined using histological samples comprising cancer cells, in particular fixed cancer tissue samples such as formalin-fixed, paraffin-embedded samples. The immunohistochemical assay used for determining the EGFR overexpression preferably includes (i) contacting the sample comprising the cancer cells with a primary antibody against EGFR, followed by (ii) contacting the sample with a secondary antibody which is directed against the primary antibody and is coupled to a visualization agent such as an enzyme which catalyzes a reaction having a visible end product, for example horseradish peroxidase. A suitable EGFR immunohistochemistry kit is the EGFR pharmDx kit (DakoCytomation NS). EGFR positive neoplastic diseases also include cancers which are positive for EGFR gene amplification as determined by fluorescence in-situ hybridization (FISH), silver in-situ hybridization (SISH) or chromogene in-situ hybridization (CISH). A cancer is positive for EGFR gene multiplication according to the FISH assay if the number of copies of the EGFR gene in the tumor cells is at least 2-times the number of copies of chromosome 17 or if the tumor cells comprise at least 4 copies of the EGFR gene. In certain embodiments, a cancer is positive for EGFR gene multiplication according to the CISH assay if at least 5 copies of the EGFR gene per cell nucleus are present in at least 50% of the tumor cells.

By “metastasis” or “metastases” is meant the spread of cancer cells from its original site (e.g. primary tumor site) to another part of the body. It is not distinguished between singular and plural of “metastasis” except were the context indicates otherwise. As described above in the background of the invention, the formation of metastasis is a very complex process and normally involves detachment of cancer cells from a primary tumor, entering the body circulation and settling down to grow within normal tissues elsewhere in the body. For details, it is referred to the respective disclosure which also applies here. As described herein, the fucose reduced anti-EGFR antibody of the invention can be used to treat metastases. Metastases can be seen as an embodiment of an EGFR positive neoplastic disease. The EGFR status of a metastasis can differ from the EGFR status of the primary cancer from which the metastasis derived (see e.g. Scartozzi et al, J Clin Oncol, 2004 22 (23):4772-8). According to a preferred embodiment, the metastasis to be treated is EGFR positive. Preferably, the primary cancer and the metastasis is EGFR-positive. Examples of EGFR positivity and methods for determining the EGFR status are described above; it is referred to the above disclosure which also applies here. According to a preferred embodiment, the EGFR positive cancer to be treated is a metastatic cancer. Examples of EGFR positive cancers were described above. The metastases can be distant metastases. Specific types of metastases that can be treated with the anti-EGFR antibodies of the invention are lymphnode metastases and visceral metastases. “Visceral metastasis” or “visceral metastases” in particular refers to metastases in the viscera, the internal organs of the body, specifically those within the chest such as heart or lungs or the abdomen, such as the liver, pancreas or intestines. In particular, the term “visceral metastasis” refers to metastases in the lung and/or the liver. Specific types of metastases that can be successfully treated with the reduced fucose anti-EGFR antibody as described herein are mesothelial metastases including pleural and peritoneal metastases, and lung metastases. “Mesothelial metastases” refer to the growth of cancer cells in or at a mesothelium such as the pleura and the peritoneum. In particular, mesothelial metastases can lead to accumulation of fluid in the cavity surrounded by the mesothelium, in particular pleural and/or abdominal effusion, e.g. due to inflammatory reaction and/or increased permeability of the affected mesothelium caused by the metastases. Other types of metastases occurring with EGFR positive diseases include skin metastases, brain metastases and bone metastases.

The term “failed treatment” or “treatment failure” or related terms according to the invention particularly refer to a treatment of cancer which results in progression of the disease. In certain embodiments, progression of disease refers to one or more of the following (i) the further growth of an existing tumor, in particular by at least 20%; (ii) the growth or the formation of one or more new lesions or metastases of an existing type; (iii) the formation of one or more further metastases of a different type; (iv) the formation of further lesions and/or (v) the increase of the size of one or more lesions. The further growth of a tumor in particular refers to an increase in tumor volume by at least 20%. The increase of the size of a lesion in particular refers to an increase in lesion size by at least 20%. The above mentioned criteria indicating a treatment failure are, however, non-limiting. Depending on the disease to be treated and/or the clinical situation also other deteriorations can be considered a treatment failure. E.g. a symptomatic deterioration that results in that a previous treatment had to be discontinued can be considered a treatment failure. As is shown by the examples, the anti-EGFR antibodies of the invention can be successfully used after other anti-cancer treatments, in particular treatment with other anti-EGFR antibodies, failed. In certain embodiments, treatment failure is determined according to the criteria of RECIST (response evaluation criteria in solid tumors).

The term “successful treatment” or “treatment success” or related terms according to the invention particularly refer to treatments of EGFR positive cancer or metastases which result in a stabilization of the disease, a partial remission and/or a full remission of the disease. A successful treatment may include one or more of the following (i) the inhibition of tumor growth or prevention of tumor growth by more than 20%; (ii) the reduction of tumor size; (iii) the prevention of further metastases of the same type and/or of a different type; (iv) the reduction of the number of metastases; (v) the prevention of further lesions; (vi) the reduction of the number of lesions; (vii) the reduction of the size of one or more lesions; (viii) the reduction in the concentration of one or more tumor markers, in particular CEA and/or Ca19-9, in the patient's circulation; (ix) the reduction of an effusion, in particular a pleural and/or abdominal effusion, and/or (x) the reduction of pain. A successful treatment may be achieved if one or more of these criteria are fulfilled. In certain embodiments, a successful treatment also includes treatments wherein a small increase in tumor size (up to about 20%), a small increase in the number of metastases or lesions or in their size (up to 20%), a small increase in the concentration of one or more tumor markers (up to 20%), or a small increase in the amount of effusion fluid (up to 20%) is observed. The reduction of tumor size in certain embodiments refers to a decrease in tumor volume by at least 30%, including a remission wherein the tumor volume is reduced by 30 to 50%, a remission wherein the tumor volume is reduced by more than 50%, and a complete remission wherein the tumor volume is reduced by 100% or is reduced to a not measurable or not detectable size. The reduction of the size of a lesion in particular refers to a decrease in the lesion size by at least 30%, including a reduction wherein the lesion size is reduced by 30 to 50%, a reduction wherein the lesion size is reduced by more than 50%, and a complete reduction wherein the lesion size is reduced by 100% or is reduced to a not measurable or not detectable size. A lesion in particular refers to a lesion caused by a primary tumor and/or by one or more metastases. The reduction in the concentration of one or more tumor markers, in particular CEA and/or Ca19-9, in the patient's circulation preferably refers to a reduction by at least 10%, more preferably at least 25% or at least 50%, most preferably at least 75%. In particular, a complete reduction of the tumor markers refers to a reduction to the normal level of healthy individuals. The reduction of an effusion in particular refers to a decrease in the amount of effusion fluid by at least 25%, including a reduction wherein the amount of effusion fluid is reduced by 25 to 50%, a reduction wherein the amount of effusion fluid is reduced by more than 50%, and a complete reduction wherein no abnormal amount of fluid can be detected in the affected body cavity such as the peritoneal cavity or the pleural cavity. A successful treatment in particular also includes treatments which result in an increase in progression-free survival and/or an increase in lifespan, in particular a progression-free survival or a remaining lifespan of at least 2 months, preferably at least 3 months, at least 4 months, at least 6 months, at least 9 months or at least 1 year, even more preferably of at least 1.5 years, at least 2 years, at least 3 years, at least 4 years or at least 5 years, or an increase in progression-free survival or remaining lifespan of at least 2 months, preferably at least 3 months, at least 4 months, at least 6 months, at least 9 months or at least 1 year, even more preferably of at least 1.5 years, at least 2 years, at least 3 years, at least 4 years or at least 5 compared to no treatment or standard treatment using high fucose anti-EGFR antibodies. A “stable disease” and accordingly a stabilization of the disease in certain embodiments includes (i) a variation in the tumor and/or metastases volume by less than 30% and/or (ii) a variation in the size of target lesions by less than 30%. The successful treatment preferably is determined for an observation period of at least 1 month, more preferably at least 2 months, at least 3 months, at least 4 months, at least 6 months, at least 9 months or at least 1 year, even more preferably at least 1.5 years, at least 2 years, at least 3 years, at least 4 years or at least 5 years. In certain embodiments, treatment success is determined according to the criteria of RECIST (response evaluation criteria in solid tumors).

The tumor marker CEA (carcinoembryonic antigen) is a glycoprotein involved in cell adhesion. The normal value of this tumor marker in the serum of healthy individuals is below 2.5 ng per mL in nonsmokers and below 5 ng per mL in smokers. In patients with metastatic cancer the tumor maker may rise to levels of above 100 ng per mL. With CEA levels of 10 ng per mL or higher a benign disease is unlikely and a malignant disease has to be expected. CA19-9 (carbohydrate antigen 19-9, also called cancer antigen 19-9 or sialylated Lewis (a) antigen) is an intercellular adhesion molecule. In healthy individuals, the normal value of CA19-9 is below 37 units per mL serum. At level above 1,000 units per mL a benign disease is unlikely and a malignant disease has to be expected. The tumor markers CEA and CA19-9 as well as other standard tumor markers can be determined in blood samples of patients using commercially available diagnosis kits and/or in clinical laboratories. In several patients included in the clinical studies described in the examples, a dramatic reduction in tumor markers, especially CEA and CA19-9, was observed, thereby demonstrating the therapeutic effect of the anti-EGFR antibody of the invention.

Treatment failure as well as a successful treatment is established based on the medical judgment of a practitioner ascertained by the results from clinical and laboratory data that are generally known in the art to assess patient treatment. Such data may be obtained by way of example, from clinical examination, cytological and histological techniques, endoscopy and laparoscopy, ultrasound, CT, PET and MRI scans, chest x-ray, mammography, and combinations thereof. Furthermore, RECIST criteria may be used to determine the tumor response.

The term “surgery” according to the invention in particular refers to a surgical removal (resection or ectomy) of tissue comprising all or a part of a tumor, in particular a primary tumor such as a breast tumor, and/or one or more metastases.

An “adjuvant therapy” in particular refers to the treatment of cancer after surgery.

A “neoadjuvant therapy” in particular refers to the treatment of cancer prior to surgery.

A “palliative therapy” in particular refers to a cancer therapy that is given specifically to address symptom management without expecting to significantly reduce the cancer. Palliative care is directed to improving symptoms associated with incurable cancer. The primary objective of palliative care is to improve the quality of the remainder of a patient's life. Pain is one of the common symptoms associated with cancer. Approximately 75% of terminal cancer patients have pain. Pain is a subjective symptom and thus it cannot be measured using technological approaches. The majority of cancer patients experience pain as a result of tumor mass that compresses neighboring nerves, bone, or soft tissues, or from direct nerve injury (neuropathic pain). Pain can occur from affected nerves in the ribs, muscles, and internal structures such as the abdomen (cramping type pain associated with obstruction). Many patients also experience various types of pain as a direct result of follow-up tests, treatments (surgery, radiation, and chemo-therapy) and diagnostic procedures (i.e., biopsy). A therapeutically useful palliative therapy is able to reduce pain.

The term “radiotherapy”, also known as radiation therapy, particularly means the medical use of ionizing radiation to control or kill malignant cells. Radiotherapy may be used in combination with surgery, as adjuvant and/or neoadjuvant therapy, or without surgery, for example to prevent tumor recurrence after surgery or to remove a primary tumor or a metastasis.

The term “pharmaceutical composition” and similar terms particularly refers to a composition suitable for administering to a human, i.e., a composition containing components which are pharmaceutically acceptable. Preferably, a pharmaceutical composition comprises an active compound or a salt thereof together with a carrier, diluent or pharmaceutical excipient such as buffer, or tonicity modifier. According to one embodiment, the pharmaceutical composition does not comprise a preservative.

The terms “antibody composition” and “composition comprising an antibody” are used interchangeably herein if the context does not indicate otherwise. Also the term “antibody” as used herein may in certain embodiments refer to an antibody composition. The antibody composition may be a fluid or solid composition, and also includes lyophilized or reconstituted antibody compositions. Preferably a fluid composition is used, more preferably an aqueous composition. In certain embodiments, it further comprises a solvent such as water, a buffer for adjusting and maintaining the pH value, and optionally further agents for stabilizing the antibody or preventing degradation of the antibody. The antibody composition preferably comprises a reasonable amount of antibodies, in particular at least 1 fmol, preferably at least 1 pmol, at least 1 nmol or at least 1 μmol of the antibody. A composition comprising a specific antibody may additionally comprise one or more further antibodies of a different amino acid sequence. In certain embodiments, the one or more different antibodies bind to a different epitope or different targets. In one embodiment, a composition comprising a specific antibody such as a reduced fucose anti-EGFR antibody according to the invention does not comprise other antibodies apart from the specific antibody. In certain embodiments, at least 75%, preferably at least 80%, at least 85%, at least 90%, at least 95%, at least 97%, at least 98% or at least 99%, most preferably about 100% of the antibodies in an antibody composition are directed to or bind to the same antigen or epitope or have the same amino acid sequence. Accordingly, in certain embodiments the term “reduced fucose anti-EGFR antibody” refers to an antibody composition that is substantially free of other antibodies having different antigenic specificities and/or a different amino acid sequence. In certain embodiments, the antibody composition is a pharmaceutical composition.

### DETAILED DESCRIPTION OF THE INVENTION

The remarkable therapeutic effects achieved with the anti-EGFR antibodies of the present invention and the novel treatment opportunities provided by the present invention were briefly described in the summary of the present invention to which it is referred. The present invention provides an anti-EGFR antibody with a glycosylation site in the CH2 domain, wherein only 50% or less, 40% or less, 30% or less, preferably 25% or less, more preferably 20% to 0% of the glycans attached to said glycosylation site carry fucose (reduced fucose anti-EGFR antibody) for treating a patient with an EGFR positive neoplastic disease, in particular advanced or metastasizing cancer. Thus, the present invention provides reduced fucose anti-EGFR antibodies for treating a human patient with an EGFR positive cancer. Here, the present invention in particular provides novel treatments.

As demonstrated by the experimental data, in contrast to prior art anti-EGFR antibodies, the anti-EGFR antibody according to the present invention induces remarkably few and only mild adverse skin reactions in the treated patients. In particular, only about 40% of the patients treated with the reduced fucose anti-EGFR antibody according to the invention develop an adverse skin reaction. This is a reduction by half compared to conventional anti-EGFR antibodies which cause skin reactions in 80% or more of the patients. Additionally, the skin reactions observed with the reduced fucose antibodies of the invention were very mild and severe adverse skin reactions of grade 3 or higher were so far not observed during the ongoing clinical studies with the reduced fucose anti-EGFR antibody of the invention. This is a crucial characteristic of the antibody according to the invention which distinguishes it from prior art anti-EGFR antibodies which frequently induce such severe skin reactions. As described in detail in the background of the invention, severe skin reactions are a great burden for the patients. In many cases, the adverse skin reactions become so severe that the antibody administration has to be delayed, the dosage has to be reduced or even a termination of the treatment is clinically indicated. These problems are overcome with the reduced fucose anti-EGFR antibody of the invention. Hence, the treatment of EGFR positive neoplastic diseases with the reduced fucose anti-EGFR antibodies is much better tolerated by the patients and novel treatment opportunities are provided because of the unique characteristics of the reduced fucose anti-EGFR antibodies of the invention. In particular, the reduced fucose anti-EGFR antibodies according to the invention enable the treatment of EGFR-positive neoplastic diseases in patients who could not or can no longer be treated with conventional anti-EGFR antibodies due to adverse skin reactions caused by said conventional anti-EGFR antibodies. This in particular applies to patients who underwent a therapy with a conventional anti-EGFR antibody wherein said treatment had to be interrupted or discontinued because of adverse skin reactions, in particular severe adverse skin reactions of grade 3 or higher or wherein the dosage of the conventional anti-EGFR antibody had to be reduced because of adverse skin reactions, thereby jeopardizing the therapeutic efficacy. Furthermore, as only mild adverse skin reactions are observed with the reduced fucose anti-EGFR antibody of the invention, higher dosages can be used if desired. Hence, the present invention provides novel and improved treatments for a human patient afflicted with an EGFR positive neoplastic disease, in particular malignant epithelial tumors, wherein the patient suffers from, previously suffered from or is at risk of suffering from an adverse skin reaction against an EGFR inhibitor, in particular anti-EGFR antibodies such as Erbitux®, panitumumab, zalutumumab or GA201.

Even though the reduced fucose anti-EGFR antibody according to the present invention causes less and only mild adverse skin reactions, it demonstrated in clinical trials high therapeutic efficacy in the treatment of several different EGFR positive cancer types, including but not limited to colorectal cancers, renal cancers, lung cancer, gastric cancer and esophageal cancer. In the ongoing clinical studies, a clear therapeutic benefit for the patients was observed in a high number of patients, including long term stable disease, partial and even complete responses, and these significant therapeutic benefits were observed already at low antibody dosages (see Examples, below). This is especially remarkable since it was believed in the art that the therapeutic efficacy of EGFR inhibitors such as anti-EGFR antibodies is directly correlated with the severity of adverse skin reactions caused by the treatment. This dogma is surprisingly disproved by the reduced fucose anti-EGFR antibody according to the present invention which is therapeutically highly effective and still shows remarkably reduced and only mild incidences of adverse skin reactions over a broad dosage range.

A further important finding is that the reduced fucose anti-EGFR antibody according to the invention is highly effective against different forms of renal cancer. Renal cell carcinomas are generally considered not to be susceptible to chemotherapy or antibody-based therapy. The clinical studies, however, demonstrate that different types of renal cell carcinomas, including clear cell as well as non-clear cell renal cell carcinomas, can effectively be treated by the reduced fucose anti-EGFR antibody according to the invention. Thereby, novel treatments are provided for a patient afflicted with a renal cancer, in particular being afflicted with a clear cell or non-clear cell renal carcinoma. The treatment with the reduced fucose anti-EGFR antibody is in particular suitable if said patient has been previously treated with an EGFR inhibitor, in particular an anti-EGFR antibody, and wherein said previous treatment was interrupted, terminated or wherein the dosage of the EGFR inhibitor had to be reduced because an adverse skin reaction occurred during treatment. The anti-EGFR antibody according to the invention can in particular be used for treating a patient afflicted with a renal cancer, in particular being afflicted with a clear cell or non-clear cell renal carcinoma, with known severe skin reaction of grade 3 or 4 against an anti-EGFR antibody which causes such severe skin reactions, in particular cetuximab (Erbitux®) or panitumumab.

As another important feature, treatment with the reduced fucose anti-EGFR antibody according to the invention significantly reduces malignant effusions in the treated cancer patients and even achieved a complete removal of the effusions in patients. Malignant effusions are complications associated with a cancer disease and cause the patients great discomfort and pain (see above). As demonstrated in the clinical studies, the present anti-EGFR antibody is able to alleviate these complications and improve the general situation and health condition of the treated patients—in addition to the effective treatment of the cancer. The treatment with the reduced fucose anti-EGFR antibody is in particular suitable if said patient has been previously treated with an EGFR inhibitor, in particular an anti-EGFR antibody, and wherein said previous treatment was interrupted, terminated or wherein the dosage of the EGFR inhibitor had to be reduced because an adverse skin reaction occurred during treatment. The anti-EGFR antibody according to the invention can in particular be used for treating a patient afflicted with a malignant effusion with known severe skin reaction of grade 3 or 4 against an anti-EGFR antibody which causes such severe skin reactions, in particular cetuximab (Erbitux®) or panitumumab.

Furthermore, the treatment with the reduced fucose anti-EGFR antibody according to the invention is also not hampered by specific characteristics of the patient or tumor which pose problems for conventional anti-EGFR antibodies. In particular, the antibody according to the invention shows consistently good therapeutic efficacy regardless of whether the tumor comprises a KRAS mutant or not and regardless of the FcγRIIIa allotype of the patient. These are further advantageous characteristics of the reduced fucose anti-EGFR antibody of the invention. The conventionally used anti-EGFR antibody Erbitux®, for example, is not suitable for treatment of KRAS mutant tumors and indeed is only approved for treatments of KRAS wildtype cancer. Additionally, high fucose anti-EGFR antibodies such as Erbitux® have a highly reduced ADCC activity in patients having an unfavorable FcγRIIIa allotype and thus show reduced efficacy in patients that are homozygous for phenylalanine in amino acid position 158 of the Fcγ receptor IIIa (FcγRIIIa-158F/F) or in patients that are heterozygous for valine and phenylalanine in amino acid position 158 of the Fcγ receptor IIIa (FcγRIIIa-158V/F).

Hence, the present invention provides reduced fucose anti-EGFR antibodies with a greatly improved therapeutic profile and provides novel therapeutic treatments with said antibodies, in particular novel patient groups.

Preferred embodiments of the anti-EGFR antibody of the invention and the individual treatments are described subsequently and in the claims to which it is referred.

### Reduced Fucose Anti-EGFR Antibody According to the Invention

As described above, an important characteristic of the reduced fucose anti-EGFR antibody of the invention is the observed significant reduction of adverse skin reactions. Adverse skin reactions that are commonly caused by prior art anti-EGFR antibodies include but are not limited to rash and/or desquamation such as acneiform rash, erythema multiforme and hand-foot skin reaction; pruritus; itching; nail changes; and ulceration (see also above). An overview over adverse skin reactions as well as their classification and grading is given in the Common Terminology Criteria for Adverse Events of the Cancer Therapy Evaluation Program Version 3.0 of the U.S. National Institute of Health to which it is referred. While prior art anti-EGFR antibodies frequently cause adverse skin reactions in the patients (e.g. ˜80% or even ˜90% of the patients in case of Erbitux®), adverse skin reactions occur less frequently with the reduced fucose anti-EGFR antibody of the invention. Thus, according to one embodiment, the reduced fucose anti-EGFR antibody of the invention causes adverse skin reactions (which include any grade) in not more than 80%, not more than 75%, not more than 70%, not more than 65%, not more than 60%, preferably not more than 55% and more preferably not more than 50% of the treated patients.

As described above, particularly problematic with prior art anti-EGFR antibodies is the occurrence of adverse skin reactions of grade 3 or 4 (for grading see Common Terminology Criteria for Adverse Events of the Cancer Therapy Evaluation Program Version 3.0 of the U.S. National Institute of Health) as here, an adaption and sometimes even interruption of the treatment is clinically indicated. In particular problematic are skin rashes of grade 3 or 4. Skin rash of grade 3 include e.g. severe, generalized erythroderma, macular, papular or vesicular eruption and/or desquamation covering at least 50% of the body skin area. Skin rash of grade 4 include e.g. generalized exfoliative, ulcerative, and/or bullous dermatitis. Acneiform rash of grade 3 or higher is particularly associated with pain, disfigurement, ulceration, and/or desquamation. The health issues that occur due to such adverse skin reactions of grade 3 or higher were described in detail above and there is a great demand for novel treatment options for such patients that encountered adverse skin reactions of grade 3 or higher in previous treatments or which have a risk that such an adverse skin reaction will occur during treatment with a conventional anti-EGFR antibody such as e.g. Erbitux®. Such novel treatments are provided by the present disclosure.

In one aspect of the invention, the reduced fucose anti-EGFR antibody has the characteristic that it causes adverse skin reactions of grade 3 or higher in not more than 20%, not more than 19%, not more than 18%, not more than 17%, not more than 16%, not more than 15%, not more than 14%, not more than 13%, not more than 12%, not more than 11%, preferably not more than 10%, not more than 9%, not more than 8%, not more than 7%, not more than 6%, more preferably not more than 5%, not more than 4%, not more than 3%, not more than 2.5%, not more than 2%, not more than 1.5%, not more than 1.25%, not more than 1%, not more than 0.75%, not more than 0.5%, or not more than 0.25% of the treated patients. In the ongoing clinical studies, no adverse skin reactions of grade 3 or higher were yet observed. In particular, no treatment interruption, dosage reduction or delays in administration were clinically indicated because of adverse skin reactions during the study.

In certain embodiments, the reduced fucose anti-EGFR antibody causes skin rash and/or acneiform rash in not more than 70%, not more than 65%, not more than 60%, not more than 55%, preferably not more than 50%, not more than 45%, not more than 40%, not more than 35%, more preferably not more than 30%, not more than 25%, or not more than 20% of the treated patients. According to one embodiment, the reduced fucose anti-EGFR antibody causes acneiform skin rash of grade 3 or higher in not more than 20%, preferably not more than 19%, not more than 18%, not more than 17%, not more than 16%, not more than 15%, not more than 14%, not more than 13%, not more than 12%, not more than 11%, preferably not more than 10%, not more than 9%, not more than 8%, not more than 7%, not more than 6%, more preferably not more than 5%, not more than 4%, not more than 3%, not more than 2.5%, not more than 2%, not more than 1.5%, not more than 1.25%, not more than 1%, not more than 0.75%, not more than 0.5%, or not more than 0.25% of the treated patients. Acneiform skin rash as it is commonly associated with prior art antibodies in particular is associated with pain, disfigurement, ulceration, and/or desquamation. As shown by the clinical data provided in the application, the reduced fucose anti-EGFR antibody according to the invention causes a low incidence of acneiform rash (of any grade, i.e. including grade 1 and 2) of only approx. 20% in the treated patients. Observed acneiform rashes were only mild and in particular no acneiform rash of grade 3 or higher occurred so far. The terms “acneiform rash” or “acneiform skin rash” as used herein in particular also includes and refers to “acneiform dermatitis”.

In certain embodiments, the reduced fucose anti-EGFR antibody causes hypomagnesemia and/or hypokalemia in not more than 30%, not more than 20%, not more than 15%, not more than 12%, not more than 10% not more than 9%, not more than 8%, or not more than 7% of the treated patients. According to one embodiment, the reduced fucose anti-EGFR antibody causes hypomagnesemia and/or hypokalemia of grade 3 or higher in not more than 10%, preferably not more than 9%, not more than 8%, not more than 7%, not more than 6%, not more than 5%, not more than 4%, not more than 3%, not more than 2%, not more than 1.5%, not more than 1%, not more than 0.75%, not more than 0.5%, or not more than 0.25% of the treated patients. In certain embodiments, the reduced fucose anti-EGFR antibody causes diarrhea in not more than 40%, not more than 35%, not more than 30%, not more than 27% not more than 25%, not more than 23%, or not more than 22% of the treated patients. According to one embodiment, the reduced fucose anti-EGFR antibody causes diarrhea of grade 3 or higher in not more than 15%, preferably not more than 12%, not more than 10%, not more than 7%, not more than 6%, not more than 5%, not more than 4%, not more than 3%, not more than 2%, not more than 1.5%, not more than 1%, not more than 0.75%, not more than 0.5%, or not more than 0.25% of the treated patients.

In certain embodiments, the reduced fucose anti-EGFR antibody causes the adverse reactions discussed herein in no more than the indicated percentage of the treated patients when administered in an amount of at least 10 mg per dose, preferably at least 50 mg per dose, at least 100 mg per dose, at least 200 mg per dose, at least 240 mg per dose, at least 300 mg per dose, at least 350 mg per dose, at least 400 mg per dose, at least 500 mg per dose, at least 600 mg per dose or at least 700 mg per dose. The reduced fucose anti-EGFR antibody is administered for at least 4 doses, preferably at least 6 doses, at least 8 doses or at least 10 doses, in particular at least every third week, preferably at least every second week or at least weekly. In certain embodiments, the reduced fucose anti-EGFR antibody causes the adverse reactions discussed herein in no more than the indicated percentage of the treated patients when administered for at least 4 weeks, in particular at least 6 weeks, at least 8 weeks or at least 10 weeks, in an average amount of at least 200 mg per week, in particular at least 240 mg per week, at least 300 mg per week, at least 350 mg per week, at least 400 mg per week, at least 500 mg per week, at least 600 mg per week or at least 700 mg per week.

In this respect, the present invention further provides a method for reducing the adverse reactions in a treatment of a patient having an EGFR positive neoplastic disease with an EGFR inhibitor, comprising the step of treating the patient with the reduced fucose anti-EGFR antibody according to the invention. In particular, the method includes terminating the treatment with the EGFR inhibitor and starting a treatment with the reduced fucose anti-EGFR antibody according to the invention. Preferably, the adverse reactions are reduced to a level as described herein, in particular as described above. For example, the severity of one or more types of adverse reactions may be reduced and/or the occurrence of one or more types of adverse reactions may be prevented. Preferably, the severity or grade of an adverse skin reaction, in particular rash or acneiform rash, is reduced. In certain embodiments, the patient suffers from an adverse reaction of grade 3 or higher or grade 4 or higher, in particular an adverse skin reaction such as rash or acneiform rash, of grade 3 or higher or grade 4 or higher, and the grade of said adverse reaction is reduced to 2 or lower, preferably to 1, or said adverse reaction is completely removed because of the treatment with the anti-EGFR antibody of the invention. In certain embodiments, the reduced fucose anti-EGFR antibody is preferably administered to the patient in high dosages. In particular, treating the patient with the reduced fucose anti-EGFR antibody comprises administering to the patient the reduced fucose anti-EGFR antibody in an amount of at least 200 mg per dose, preferably at least 500 mg per dose, at least 700 mg per dose, or at least 900 mg per dose. The reduced fucose anti-EGFR antibody is preferably administered to the patient in intervals of between one dose every 5 days to one dose every 3 weeks, more preferably every week or every second week. In certain embodiments, the patient suffered from acneiform skin rash, in particular acneiform skin rash grade 3 or higher, caused by the EGFR inhibitor used in the previous treatment. The EGFR inhibitor in particular causes adverse skin reactions in 70% or more, 80% or more or 90% or more of the treated patients. In certain embodiments, the EGFR inhibitor causes adverse skin reactions of grade 3 or higher in 25% or more, 30% or more or 35% or more of the treated patients. Furthermore, the EGFR inhibitor may cause acneiform skin rash in 60% or more, 70% or more or even 80% or more of the treated patients, in particular acneiform skin rash of grade 3 or higher in 25% or more, 30% or more or even 35% or more of the treated patients. In certain embodiments, the EGFR inhibitor used in the previous treatment is an anti-EGFR antibody such as cetuximab (Erbitux®), panitumumab (Vectibix®), zalutumumab and GA201. All features and embodiments described herein for the reduced fucose anti-EGFR antibody also apply to the method according to the invention.

The presence or absence of adverse skin reactions caused by an anti-EGFR antibody is preferably determined at least 2 days, more preferably at least 3 days or at least 4 days after the administration of the anti-EGFR antibody. In preferred embodiments, only adverse skin reactions which are present for at least 2 days, preferably for at least 3 days or at least 4 days, are considered for determining the presence or absence of adverse skin reactions caused by the anti-EGFR antibody. For determining the adverse skin reactions caused by the anti-EGFR antibody, preferably only adverse skin reactions occurring in an interval of from 2 days after the first administration of the anti-EGFR antibody to 3 weeks after the last administration of the anti-EGFR antibody, more preferably in an interval of from 3 days after the first administration to 2 weeks after the last administration, or from 4 days after the first administration to 1 week after the last administration are considered. Thereby, only skin reactions caused by the biological and/or therapeutic activity of the anti-EGFR antibody are considered, while skin reactions caused by the infusion of the pharmaceutical composition (infusion-related reaction (IRR)) are excluded. The adverse reactions, in particular the adverse skin reactions, as referred to herein, in particular do not include infusion-related reactions.

It is believed that the low occurrence of adverse skin reactions in patients treated with the reduced fucose anti-EGFR antibody of the invention which are also milder compared to prior art antibodies is due to an inhibition or a reduced induction of granulocyte migration into the patient's skin, resulting in a low concentration of granulocytes in the skin region. Without being bound to any theory, the optimized Fc glycosylation of the reduced fucose anti-EGFR antibody, which may also influence the binding of immune cells, in particular white blood cells such as granulocytes, may be responsible for this effect (see below).

It was found that the reduced fucose anti-EGFR antibody of the invention, in particular having the preferred glycosylation characteristics as described herein, shows differences in its interactions with immune cells in comparison to other anti-EGFR antibodies, such as the high fucose anti-EGFR antibody Erbitux®. For example, it was found that almost all granulocytes and a large number of the NK cells present in a human blood sample are bound by the reduced fucose anti-EGFR antibody of the invention. In contrast, high fucose cetuximab (Erbitux®) only binds less than 5% of the granulocytes and NK cells in the sample (see Example 7). In this respect, also a reduction of circulating NK cells after treatment with the anti-EGFR antibody according to the invention was observed in all tested patients of the clinical study, indicating a stimulation and migration of NK cells into the affected tumor tissue. Without being bound to any theory, it is believed that the altered immune cell binding of the reduced fucose anti-EGFR antibody of the invention is associated with or even responsible for and at least reflects the reduced occurrence of adverse skin reactions. In preferred embodiments, the reduced fucose anti-EGFR antibody is capable of binding to at least 60%, preferably at least 80% of human granulocytes or a certain type of human granulocytes, in particular CD66 positive human granulocytes, in a human blood sample. In certain embodiments, the reduced fucose anti-EGFR antibody binds to human granulocytes at least 10-fold stronger, preferably at least 20-fold stronger than the high fucose anti-EGFR antibody Erbitux®. Furthermore, in some embodiments, the reduced fucose anti-EGFR antibody is capable of binding to at least 10%, preferably at least 20% of human NK cells, in particular CD16 and CD56 positive human NK cells in a human blood sample. In particular, the reduced fucose anti-EGFR antibody binds to human NK cells at least 5-fold stronger, preferably at least 10-fold stronger than the high fucose anti-EGFR antibody Erbitux®. The binding behavior can be determined in appropriate assays. For these binding assays, preferably a human blood sample is contacted with the reduced fucose anti-EGFR antibody at a concentration of about 10 μg/ml and the binding of the antibody to the blood cells is detected using a secondary antibody carrying a detectable signal such as a fluorophore. Binding to the different blood cells can be determined as described in Example 7, below. Furthermore, a fast and strong stimulation of chemokine IP-10 was determined in all analyzed patients treated with the anti-EGFR antibody of the invention. This indicates involvement of macrophages in the response to the reduced fucose anti-EGFR antibody of the invention. IP-10 is secreted by macrophages upon their stimulation and hence is a marker for macrophage activity.

These findings demonstrate that the reduced fucose anti-EGFR antibody according to the present invention is capable of attacking cells of the EGFR positive neoplastic disease (target cells) via different mechanisms of action.

Upon binding to the EGFR on the target cell's surface, the reduced fucose anti-EGFR antibody is capable of inducing antibody-dependent cellular cytotoxicity (ADCC). In particular, the reduced fucose anti-EGFR antibody binds to and activates natural killer cells (NK cells) and other immune cells via their Fcγ receptor III, in particular Fcγ receptor IIIa. The activated NK cells release cytokines such as IFN-γ, and/or cytotoxic granules containing perforin and/or granzymes that enter the target cell and promote cell death by triggering apoptosis or cell lysis. Hence, the target cells are destroyed by the reduced fucose anti-EGFR antibody via induction of ADCC.

In certain embodiments, the reduced fucose anti-EGFR antibody is capable of reducing cell proliferation and/or inducing cell death or apoptosis by blocking the EGFR on the target cells. In certain target cells the EGFR signaling is required for proliferation and/or survival. Upon binding of the reduced fucose anti-EGFR antibody to the EGFR on the target cell's surface, ligand binding and/or activation of the EGFR is inhibited. Furthermore, the EGFR bound by the antibody is internalized by the target cell, thereby reducing the EGFR amount on the surface of the cell. By these mechanisms, the activation of the EGFR signaling pathway, which leads to cell survival, proliferation and inhibition of apoptosis, is reduced or even prevented. As direct reaction of the target cells on the binding of the reduced fucose anti-EGFR antibody to EGFR, the cells do not proliferate and apoptosis is induced.

In a further mechanism of action, the reduced fucose anti-EGFR antibody is capable of binding to and activating granulocytes at the site of the neoplastic disease. The activated granulocytes release granules and thereby induce apoptosis, lysis and/or necrosis in the target cells. Strong binding and activation of granulocytes by the reduced fucose anti-EGFR antibody was demonstrated in the examples. In particular, the production of oxidative species and lactoferrin, components of the granules of activated granulocytes, was observed upon incubation of whole blood samples with the reduced fucose anti-EGFR antibody according to the present invention in the presence of target cells. Thus, the reduced fucose anti-EGFR antibody is in embodiments capable of attacking the target cells via activation of granulocytes, in particular neutrophil and eosinophil granulocytes. In certain embodiments, the treatment of the EGFR positive neoplastic disease with the reduced fucose anti-EGFR antibody involves binding and activation of granulocytes, in particular neutrophil and eosinophil granulocytes, by the reduced fucose anti-EGFR antibody bound to cells of the EGFR positive neoplastic disease; and inducing said activated granulocytes to destroy said cells of the EGFR positive neoplastic disease. In one aspect of the invention, the reduced fucose anti-EGFR antibody is for inducing a granulocyte-driven immune reaction against cells of an EGFR positive neoplastic disease in a human patient. The granulocyte-driven immune reaction preferably involves binding and activation of granulocytes by the reduced fucose anti-EGFR antibody bound to said cells of the EGFR positive neoplastic disease; and inducing said activated granulocytes to destroy said cells of the EGFR positive neoplastic disease.

Furthermore, in embodiments the reduced fucose anti-EGFR antibody is capable of activating macrophages which attack and destroy the target cells. It could be demonstrated that upon administration of the reduced fucose anti-EGFR antibody the chemokine IP-10 is released in the treated patients. IP-10 is secreted by activated macrophages. The increase in IP-10 concentration in the patients is a strong indicator for macrophage activation. Hence, in these embodiments the reduced fucose anti-EGFR antibody is capable of attacking the target cells via activation of macrophages. In embodiments, the treatment of the EGFR positive neoplastic disease with the reduced fucose anti-EGFR antibody involves activation of macrophages by the reduced fucose anti-EGFR antibody bound to cells of the EGFR positive neoplastic disease; and inducing said activated macrophages to destroy said cells of the EGFR positive neoplastic disease. In one aspect of the invention, the reduced fucose anti-EGFR antibody is for inducing a macrophage-driven immune reaction against cells of an EGFR positive neoplastic disease in a human patient. The macrophage-driven immune reaction preferably involves activation of macrophages by the reduced fucose anti-EGFR antibody bound to said cells of the EGFR positive neoplastic disease; and inducing said activated macrophages to destroy said cells of the EGFR positive neoplastic disease. Destroying a cell, as referred to herein, in particular means and includes inducing apoptosis, inducing cell lysis and/or inducing necrosis of said cell.

An advantageous feature of the reduced fucose anti-EGFR antibody according to the invention is it's improved glycosylation pattern which is apparently also responsible for the remarkable therapeutic characteristics of the anti-EGFR antibody of the invention. The reduced fucose anti-EGFR antibody is an IgG antibody, preferably an IgG1 antibody, which has a glycosylation site in the second constant domain of the heavy chain (CH2). An antibody normally has two heavy chains having identical amino acid sequences. Hence, the reduced fucose anti-EGFR antibody according to the invention preferably has at least two glycosylation sites, one in each of its two CH2 domains. This glycosylation site in particular is at an amino acid position corresponding to amino acid position 297 of the heavy chain according to the Kabat numbering and has the amino acid sequence motive Asn Xaa Ser/Thr wherein Xaa may be any amino acid except proline. Details were also described above and it is referred to the above disclosure. The N-linked glycosylation at Asn297 is conserved in mammalian IgGs as well as in homologous regions of other antibody isotypes. Due to optional additional amino acids which may be present in the variable region or other sequence modifications, the actual position of this conserved glycosylation site may vary in the amino acid sequence of the antibody. In certain embodiments, in at least 80%, preferably at least 85%, at least 90% or at least 95%, more preferably in at least 98% of the reduced fucose anti-EGFR antibody comprised in a composition, the glycosylation site of at least one CH2 domain, preferably of both CH2 domains, carries a carbohydrate structure. The amount of fucosylation in the CH2 domain as described herein is determined at this glycosylation site in the Fc region (also referred to as Fc fucosylation). Preferably, at least 90%, more preferably at least 95% of the glycans attached to the reduced fucose anti-EGFR antibody are biantennary complex type N-linked carbohydrate structures, preferably comprising at least the following structure:
Asn-GlcNAc-GlcNAc-Man-(Man-GlcNAc)₂
   wherein Asn is the asparagine residue of the polypeptide portion of the antibody; GlcNAc is N-acetylglucosamine and Man is mannose. The terminal GlcNAc residues may further carry a galactose residue, which optionally may carry a sialic acid residue. A further GlcNAc residue (named bisecting GlcNAc) may be attached to the Man nearest to the polypeptide. A fucose may be bound to the GlcNAc attached to the Asn. FIG. 6 shows a schematic representation of a complex type N-linked carbohydrate structure.

The reduced fucose anti-EGFR antibody has an amount of fucose in the carbohydrate chains attached to the CH2 domain which is 50% or less, 40% or less, 30% or less or even 25% or less, more preferably 20% or less or 15% to 0%. In certain embodiments, the reduced fucose anti-EGFR antibody is even afucosylated and thus does not comprise any fucose. In preferred embodiments, however, the reduced fucose anti-EGFR antibody comprises at least a residual amount of fucose of at least 2%, at least 3% and preferably at least 5% in the Fc glycosylation. In certain embodiments, the amount of carbohydrate chains carrying fucose at the CH2 domain preferably is in the range of from 1% to 30%, more preferably from 2% to 25%, most preferably from 3% to 20% or from 4% to 15%.

Anti-EGFR antibodies having a reduced amount of fucosylation, including antibodies which do not carry any fucose, as used herein can be obtained by various means. E.g. the anti-EGFR antibody can be expressed in a host cell with altered glycosylation machinery. Cells with altered glycosylation machinery have been described in the art and can be used as host cells to produce recombinant anti-EGFR antibodies having a reduced fucosylation in their Fc region as described herein. For example, EP 1,176,195 by Hang et al. describes a cell line with a functionally disrupted FUT8 gene, which encodes a fucosyl transferase, such that antibodies expressed in such a cell line exhibit hypofucosylation. Therefore, in one embodiment, the antibodies comprised in the compositions of the invention are produced by recombinant expression in a cell line which exhibits hypofucosylation pattern, for example, a mammalian cell line with deficient expression of the FUT8 gene encoding fucosyltransferase. WO 03/035835 describes a variant CHO cell line, Lecl3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). The antibodies of the invention can be produced in yeast or filamentous fungi engineered for mammalian-like glycosylation pattern and capable of producing antibodies lacking fucose as glycosylation pattern (see for example EP 1 297 172 B1). Preferably, the reduced fucose anti-EGFR antibody is obtained by recombinant expression in a human cell line which has a reduced or even no fucosylation capacity. A respective reduced or absent fucosylation capacity can be achieved e.g. by reducing the expression of enzymes necessary for fucosylation (e.g. α1,6-fucosyltransferase encoded by the gene FUT8 or GDP-D-mannose-4,6-dehydratase (GMD) encoded by the GMD gene), or by eliminating the respective gene functions, e.g. by gene knockout. A respective effect can also be achieved using compounds that induce gene knockdown such as e.g. antisense molecules or compounds that mediate RNA interference. Compounds that mediate RNA interference include but are not limited to short interfering nucleic acids (siNA), short interfering RNA (siRNA), microRNA (miRNA), short hairpin RNAs (shRNA) as well as precursors thereof which are processed in the cell to the actual RNAi inducing compound. Suitable siRNAs targeting the chosen/identified target sequences of the target genes on the RNA level can be identified by using proper computational methods, applying certain design-algorithms. In order to obtain a siRNA against the target transcript, the double-stranded molecule can be transfected directly into the cell. Alternatively, the siRNA may result from processing by dicer, an enzyme that converts either long dsRNAs or small hairpin RNAs (shRNAs) into siRNAs. These precursors or the final siRNA molecules can be produced exogenously (artificially) and can then be introduced into the cells by various transfection methods. According to a further embodiment, the RNAi inducing compound is expressed by a vector that is transfected into the host cell. For siRNA, this can be done e.g. by the introduction of a loop between the two strands, thus producing a single transcript, which can be then processed into a functional siRNA in the host cell. According to one embodiment, such siRNA providing vector is stably integrated into the genome of the host cell.

The reduced fucose anti-EGFR antibody preferably is produced recombinantly in a human cell line, preferably a human blood cell line, in particular in a human myeloid leukemia cell line. The cell line used for producing the reduced fucose anti-EGFR antibody preferably has a reduced or absent fucosylation activity and/or the reduced fucose anti-EGFR antibody is produced under conditions which result in a reduced or even absent fucosylation of the antibody. As described herein, a reduced or absent fucosylation activity can be achieved by manipulating the expression or activity of enzymes necessary for fucosylation (e.g. FUT8 or GMD). Preferred human cell lines which can be used for production of the reduced fucose anti-EGFR antibody, in particular Fuc− cetuximab, as well as suitable production procedures are described in WO 2008/028686 A2, herein incorporated by reference. In a specific embodiment, the low fucose anti-EGFR antibody is obtained by expression in a cell line with reduced fucosylation activity which is derived from the human cell line GT-5s deposited at the DSMZ—Deutsche Sammlung von Mikroorganismen and Zellkulturen, Inhoffenstraβe 7B, 38124 Braunschweig (DE) by the Glycotope GmbH, Robert-Rössle-Str. 10, 13125 Berlin (DE) on Jul. 28, 2010 under the accession number DSM ACC 3078.

In certain embodiments, the level of fucosylation of the reduced fucose anti-EGFR antibody may be reduced after its production by the cell line, for example by in vitro treatment with a fucosidase or by selective enrichment of non-fucosylated antibodies. In certain embodiments, the level of fucosylation of the reduced fucose anti-EGFR antibody is not or not significantly reduced after its production by the cell line. In particular, according to one embodiment, the anti-EGFR antibody is not enzymatically treated in vitro in order to achieve a reduction of fucose. In certain embodiments, the glycosylation pattern of the anti-EGFR antibody is after production in the host cell not altered by an in vitro process that influences the glycosylation pattern.

In preferred embodiments, the reduced fucose anti-EGFR antibody comprises bisecting N-acetylglucosamine (bisGlcNAc). It may comprise an amount of bisGlcNAc in the carbohydrate chains attached to the CH2 domain of at least 2%, preferably at least 5% or at least 8%, more preferred at least 10%, most preferred at least 13%. The amount of bisGlcNAc preferably is in the range of from 5% to 50%, preferably from 7% to 40%, more preferably from 8% to 35% and most preferably from 10% to 30%. It was found that reducing the amount of core fucose and at the same time increasing the amount of bisGlcNAc in the Fc glycans provides a reduced fucose anti-EGFR antibody which shows a strong increase in tumor lysis, a strong anti-metastatic efficacy and furthermore, a markedly reduced adverse reaction profile.

In certain embodiments, the reduced fucose anti-EGFR antibody comprises an amount of galactosylated carbohydrate chains attached to the CH2 domain of at least 50%, preferably at least 55%, at least 60%, at least 65% or at least 70%. The amount of galactosylated carbohydrate chains preferably is in the range of from 50% to 99%, more preferably from 60% to 97%, most preferably from 65% to 95%. In particular, the reduced fucose anti-EGFR antibody preferably comprises an amount of carbohydrate chains carrying two galactose units attached to the CH2 domain of at least 10%, preferably at least 15%, at least 20 or at least 25%. The amount of carbohydrate chains carrying two galactose units at the CH2 domain preferably is in the range of from 10% to 70%, more preferably from 15% to 60%, most preferably from 20% to 50%. Furthermore, the reduced fucose anti-EGFR antibody preferably comprises an amount of sialylated carbohydrate chains attached to the CH2 domain of at least 1%, preferably at least 1.5%, at least 2% or at least 2.5%. In particular, at least 0.5%, preferably at least 1% of the carbohydrate chains attached to the CH2 domain carry two sialic acid units. The amount of carbohydrate chains carrying at least one sialic acid at the CH2 domain preferably is in the range of from 1% to 18%, more preferably from 2% to 14% and/or the amount of carbohydrate chains carrying two sialic acid at the CH2 domain preferably is in the range of from 0.5% to 6%, more preferably from 1% to 5%.

In certain embodiments, the low fucose anti-EGFR antibody according to the invention has one or more, or at least two, at least three, at least four, at least five or preferably all of the following glycosylation characteristics in the CH2 domain:
(i) a relative amount of glycans carrying a fucose residue of 30% or less, preferably 25% or less, in particular in the range of 1% to 20%;
(ii) a relative amount of glycans carrying a bisecting GlcNAc of at least 5%, preferably at least 10%;
(iii) a relative amount of glycans carrying at least one galactose of at least 50%, preferably at least 60%;
(iv) a relative amount of glycans carrying two galactoses of at least 10%, preferably at least 15%;
(v) a relative amount of glycans carrying at least one sialic acid of at least 1%, preferably at least 2%;
(vi) optionally, a relative amount of glycans carrying two sialic acids of at least 0.5%, preferably at least 1%.

In preferred embodiments, the reduced fucose anti-EGFR antibody according to the invention comprises an additional glycosylation site in its Fab fragment, in particular in the heavy chain variable region VH. In preferred embodiments, the reduced fucose anti-EGFR antibody according to the invention comprises two heavy chains having identical amino acid sequences and two light chains having identical amino acid sequences. Hence, the reduced fucose anti-EGFR antibody according to the invention in certain embodiments comprises two additional glycosylation sites, in particular one in each of its two VH domains. The carbohydrate chains attached to the Fab fragment preferably comprises a low amount of fucose and a high amount of sialic acid, galactose and bisecting GlcNAc. The amount of fucose in the carbohydrate chains attached to the VH domain preferably is 40% or less, 35% or less, 30% or less or even 25% or less, more preferably 22% or less, most preferably 20% or less. The reduced fucose anti-EGFR antibody may comprise an amount of bisGlcNAc in the carbohydrate chains attached to the VH domain of at least 30%, preferably at least 35% or at least 40%, more preferred at least 45%, most preferred at least 50%. The amount of bisGlcNAc at the VH domain preferably is in the range of from 30% to 95%, preferably from 35% to 90%, more preferably from 40% to 85% and most preferably from 45% to 80%. Furthermore, the reduced fucose anti-EGFR antibody preferably comprises an amount of galactosylated carbohydrate chains attached to the VH domain of at least 75%, preferably at least 85%, at least 90%, at least 95% or at least 97%. In particular, the reduced fucose anti-EGFR antibody preferably comprises an amount of carbohydrate chains carrying at least two galactose units attached to the VH domain of at least 60%, preferably at least 70%, at least 75%, at least 80%, at least 85% or at least 90%. The amount of carbohydrate chains carrying at least two galactose units at the VH domain preferably is in the range of from 70% to 99%, more preferably from 75% to 98%, most preferably from 80% to 97%. Furthermore, the reduced fucose anti-EGFR antibody preferably comprises an amount of sialylated carbohydrate chains attached to the VH domain of at least 50%, preferably at least 60%, at least 65%, at least 70%, at least 75% or at least 80%. In particular, at least 35%, preferably at least 40%, at least 45% or at least 50% of the carbohydrate chains attached to the VH domain carry at least two sialic acid units.

Accordingly, in certain embodiments the reduced fucose anti-EGFR antibody according to the invention comprises a glycosylation site in the heavy chain variable region and has one or more, two or more or preferably all of the following glycosylation characteristics in the VH domain:
(i) a relative amount of glycans carrying a fucose residue of 40% or less, preferably 35% or less;
(ii) a relative amount of glycans carrying a bisecting GlcNAc of at least 35%, preferably at least 40%;
(iii) a relative amount of glycans carrying at least one galactose of at least 85%, preferably at least 90%;
(iv) a relative amount of glycans carrying at least two galactoses of at least 70%, preferably at least 80%;
(v) a relative amount of glycans carrying at least one sialic acid of at least 50%, preferably at least 60%;
(vi) a relative amount of glycans carrying at least two sialic acids of at least 35%, preferably at least 45%.

In particular, these glycosylation characteristics of the VH domain are present in the reduced fucose anti-EGFR antibody according to the invention in combination with the glycosylation characteristics of the CH2 domain described above.

In some embodiments, the reduced fucose anti-EGFR antibody according to the invention comprises a glycosylation site in the CH2 domain and a glycosylation site in the VH domain and has one or more, or at least two, at least three, at least four or preferably all of the following glycosylation characteristics for the entire antibody:
(i) a relative amount of glycans carrying a fucose residue of 30% or less, preferably 25% or less;
(ii) a relative amount of glycans carrying a bisecting GlcNAc of at least 20%, preferably at least 25%;
(iii) a relative amount of glycans carrying at least one galactose of at least 60%, preferably at least 70%;
(iv) a relative amount of glycans carrying at least two galactoses of at least 30%, preferably at least 35%;
(v) a relative amount of glycans carrying at least one sialic acid of at least 10%, preferably at least 15%;
(vi) a relative amount of glycans carrying at least two sialic acids of at least 4%, preferably at least 6%.

In this embodiment, the glycans attached to the Fc part and the glycans attached to the Fab part of the antibody are considered for determining the glycosylation characteristics. Preferably, the reduced fucose anti-EGFR antibody does not comprise detectable amounts of NeuGc and/or Galα1,3-Gal in its glycosylation pattern.

A glycosylation comprising bisGlcNAc, galactose and sialic acid as described above is characteristic for a human glycosylation pattern and can be obtained by expressing the anti-EGFR antibodies in a human cell line as described above. Sialic acid as mentioned herein preferably refers to N-acetyl neuraminic acid which preferably is coupled to the galactose via an α2,6-, α2,3- or α2,8-bond. According to a preferred embodiment, the reduced fucose anti-EGFR antibody comprises detectable amounts of α2,6-coupled N-acetyl neuraminic acid (NeuAc).

As discussed above, the reduced fucose anti-EGFR antibody preferably has a human glycosylation profile as described herein. Such a profile can be obtained by expressing the anti-EGFR antibody in a human cell line, preferably a human myeloid leukemia cell line (see above description to which it is referred and Example 1). A human glycosylation profile is preferably characterized in that at least 70%, preferably at least 80%, at least 85% or more preferred by at least 90% of the carbohydrate chains attached to the reduced fucose anti-EGFR antibody are complex type glycan structures, preferably biantennary complex type glycan structures. In particular, the amount of carbohydrate chains having a high mannose type or hybrid type glycan structure attached to the reduced fucose anti-EGFR antibody is 20% or less, preferably 15% or less or 10% or less, more preferably 5% or less, most preferably about 0%.

The reduced fucose anti-EGFR antibody having a human glycosylation profile particularly does not comprise detectable amounts of N-glycolyl neuraminic acid (NeuGc) and/or Galα1,3-Gal structures. Respective glycosylation structures are found in antibodies that are produced in non-human cell lines such as rodent cell lines. Due to the human glycosylation pattern which does not include Galα1,3-Gal structures, the reduced fucose anti-EGFR antibody according to the invention can be used for treatment of patients who show an immune response and in particular produce antibodies such as IgE antibodies against Galα1,3-Gal structures. Respective antibodies can be present e.g. if the patient has been previously treated with an antibody that was produced in a rodent cell line or other cell that produces an antibody with Galα1,3-Gal structures. However, IgE antibodies against Galα1,3-Gal structures can also be present due to other characteristics and may also depend on food habits. In particular, the reduced fucose anti-EGFR antibody of the invention can be used after the patient was treated with an anti-EGFR antibody that was produced in rodent cells. For example, the conventional anti-EGFR antibody Erbitux® is produced in rodent cells and carries Galα1,3-Gal structures. Thus, Erbitux® is prone to induce immune reactions against said structure. These reactions can range from a mild form to a more extreme and life-threatening response, such as renal failure. They can also decrease the effectiveness of the treatment, or create a future reaction if the patient is given a subsequent treatment containing respective antibodies. Up to 50% of the patients treated with antibodies having a rodent glycosylation pattern develop antibodies against certain non-human glycosylation structures as mentioned above. This can be problematic, in particular during long-term treatment. The anti-EGFR antibody according to the invention having a human glycosylation pattern hence enables the treatment of patients showing an immune response, in particular a severe immune response, against non-human glycosylation structures such as NeuGc and Galα1,3-Gal structures. In clinical studies, the reduced fucose anti-EGFR antibody according to the invention did so far not cause allergic reactions against said antibody. This is an important advantage over prior art anti-EGFR antibodies. E.g. Erbitux® shows in a significant number of cases severe allergic hypersensitivity reactions based on foreign Gal-Gal structures (Chung et al, 2008, N. Engl. J. Med) which can lead to fatal anaphylactic shocks. For this reason, Erbitux® is even not used in certain regions which have a high likelihood of developing such a reaction, e.g. because of pre-existing Gal-Gal IgE antibodies. Therefore, the reduced fucose anti-EGFR antibody of the invention can be advantageously used for treating a human patient who has developed an allergic reaction against a different anti-EGFR antibody in a previous treatment. Furthermore, the reduced fucose anti-EGFR antibody of the invention can be used for treating a human patient with pre-existing Gal-Gal IgE antibodies. In addition, the anti-EGFR antibody according to the invention can be used for treatment without the need of an accompanying anti-allergic treatment such as an anti-histamine treatment, e.g. a treatment with H1 and/or H2 blockers.

The reduced fucose anti-EGFR antibody preferably is an IgG antibody, more preferably an IgG1 antibody. It has the ability of specifically binding its target epitope and the ability of binding to Fcγ receptors, in particular to the Fcγ receptor IIIa. The reduced fucose anti-EGFR antibody is capable of inducing an antibody-dependent cellular cytotoxicity (ADCC) reaction. The reduced fucose anti-EGFR antibody of the invention is capable of inducing a stronger ADCC than a high fucose anti-EGFR antibody. The high fucose anti-EGFR antibody is defined herein below. In particular, the reduced fucose anti-EGFR antibody is at least 2-fold, at least 3-fold, at least 5-fold, at least 7-fold, at least 10-fold, at least 20-fold, at least 30-fold, at least 40-fold or at least 50-fold more potent in inducing ADCC than the high fucose anti-EGFR antibody, as can be determined in in vitro ADCC assays. As is shown therein, an up to 10-50 fold improvement of ADCC anti-tumor activity was observed when comparing the Fuc− cetuximab (according to the invention) with the Fuc+ cetuximab (prior art). The higher potency in inducing ADCC preferably refers to the X-fold lower concentration of the reduced fucose anti-EGFR antibody necessary for inducing the same level of ADCC (such as ratio of lysed target cells), preferably the same specific lysis at 95% of maximal lysis of the high fucose anti-EGFR antibody, compared to the high fucose anti-EGFR antibody. For example, if the reduced fucose anti-EGFR antibody induces the same level of ADCC at a 5-fold lower concentration than the high fucose anti-EGFR antibody, then the reduced fucose anti-EGFR antibody is 5-fold more potent in inducing ADCC than the high fucose anti-EGFR antibody. As is shown by the experimental data, a 10 to 50-fold less antibody concentration was needed for the same ADCC response when using the reduced fucose anti-EGFR antibody compared to a corresponding high fucose anti-EGFR antibody. In certain embodiments, the X-fold higher potency in inducing ADCC is determined as an average of the ADCC induced for each of the different FcγRIIIa allotypes. However, in certain embodiments, the X-fold higher potency in inducing ADCC refers to ADCC induced with effector cells of donors having a specific FcγRIIIa allotype such as the FcγRIIIa-158F/F allotype, the FcγRIIIa-158V/V allotype or the FcγRIIIa-158F/V allotype. As is shown by the experimental data, a reduced fucose antibody according to the present invention shows compared to a corresponding high fucose anti-EGFR antibody generally a higher ADCC. The increase in ADCC activity for the FcγRIIIa-158F/F and FN allotypes, those allotypes for which high fucose antibodies show the least activity, can be even more remarkable than as described above. In particular, the reduced fucose anti-EGFR antibody is at least 20-fold, at least 30-fold, at least 40-fold, at least 50-fold, at least 70-fold, at least 100-fold, at least 150-fold or at least 200-fold more potent in inducing ADCC than the high fucose anti-EGFR antibody in patients having the FcγRIIIa-158F/F allotype or the FcγRIIIa-158F/V allotype or in in vitro assays for determining the ADCC activity using effector cells from donors having the FcγRIIIa-158F/F allotype or the FcγRIIIa-158F/V allotype. Due to this strong increase for the less favorable allotypes (FcγRIIIa-158F/F or FcγRIIIa-158F/V allotype) wherein the high fucose anti-EGFR antibodies only insufficiently induce ADCC, the reduced fucose anti-EGFR antibody induces ADCC at comparable level in each patient group. Therefore, the anti-EGFR antibody can effectively mediate ADCC at all ADCC receptor allotypes. The higher ADCC activity is also believed to be responsible for the effective treatment of KRAS mutant tumors which cannot be treated with the high fucose anti-EGFR antibodies such as Erbitux® known in the art.

The reduced fucose anti-EGFR antibody comprises a heavy chain variable region (VH) and a CH2 domain, more preferably the domains VH, CH1, CH2 and CH3. Furthermore, the reduced fucose anti-EGFR antibody preferably comprises a light chain variable region (VL), preferably the domains VL and VH. The reduced fucose anti-EGFR antibody may comprise two heavy chains and two light chains. It preferably is a recombinant monoclonal antibody such as a human, humanized or chimeric antibody and in certain embodiments is a chimeric antibody.

The reduced fucose anti-EGFR antibody mediates ADCC and is according to a preferred embodiment capable of specifically binding to the extracellular part of EGFR, in particular to domain III of EGFR. According to certain embodiments, it has at least one, preferably at least two, more preferably all of the following activities: (i) it is capable of blocking ligand binding to EGFR, (ii) it is capable of blocking dimerization of EGFR, (iii) it is capable of blocking activation of EGFR, in particular of the kinase activity of EGFR and/or (iv) it is capable of reducing the amount of EGFR at the cell surface, in particular by inducing internalization of EGFR into the cell. Preferably, the reduced fucose anti-EGFR antibody has all of the aforementioned characteristics. Preferably, the reduced fucose anti-EGFR antibody shows cross-specificity with the antibody cetuximab and in particular binds to the same epitope as the antibody cetuximab. Preferably, the reduced fucose anti-EGFR antibody is equivalent to cetuximab in binding and Fv mediated anti-tumor properties, however, shows increased ADCC mediated anti-tumor properties and improved side effect profile due to the improved glycosylation described herein. In preferred embodiments, the reduced fucose anti-EGFR antibody comprises the same heavy chain and preferably also the same light chain CDR sequences as cetuximab. The entire amino acid sequence of the heavy chain and preferably also of the light chain of the reduced fucose anti-EGFR antibody may be at least 85% identical, at least 90% identical, at least 95% identical or at least 97% identical to the corresponding amino acid sequences of cetuximab. Preferably, the amino acid sequences of the reduced fucose anti-EGFR antibody are derived from the corresponding amino acid sequences of cetuximab.

In certain embodiments the reduced fucose anti-EGFR antibody comprises a heavy chain variable region comprising the complementarity determining regions (CDRs) CDR-H1, CDR-H2 and CDR-H3, wherein the CDR-H1 has the amino acid sequence of SEQ ID NO: 1 and/or CDR-H2 has the amino acid sequence of SEQ ID NO: 2 and/or CDR-H3 has the amino acid sequence of SEQ ID NO: 3. Preferably, the heavy chain variable region of the reduced fucose anti-EGFR antibody comprises all three of these CDR sequences and in particular comprises the amino acid sequence of SEQ ID NO: 7 or 9. In preferred embodiments, the reduced fucose anti-EGFR antibody comprises a light chain variable region comprising the complementarity determining regions (CDRs) CDR-L1, CDR-L2 and CDR-L3, wherein the CDR-L1 has the amino acid sequence of SEQ ID NO: 4 and/or CDR-L2 has the amino acid sequence of SEQ ID NO: 5 and/or CDR-L3 has the amino acid sequence of SEQ ID NO: 6. Preferably, the light chain variable region of the reduced fucose anti-EGFR antibody comprises all three of these CDR sequences and in particular comprises the amino acid sequence of SEQ ID NO: 8 or 10. Furthermore, in certain embodiments the reduced fucose anti-EGFR antibody comprises a heavy chain variable region which comprises an amino acid sequence which is at least 85% identical, at least 90% identical or at least 95% identical to the amino acid sequences of SEQ ID NO: 7 or 9, and/or a light chain variable region which comprises an amino acid sequence which is at least 85% identical, at least 90% identical or at least 95% identical to the amino acid sequences of SEQ ID NO: 8 or 10. As described above, the reduced fucose anti-EGFR antibody preferably is equivalent to cetuximab in binding and Fv mediated anti-tumor properties.

In certain preferred embodiments, the reduced fucose anti-EGFR antibody comprises a heavy chain variable region comprising the amino acid sequence of SEQ ID NO: 7 or 9 or an amino acid sequence which is at least 80%, at least 85%, preferably at least 90%, more preferred at least 95% identical thereto, wherein the CDR1 has the amino acid sequence of SEQ ID NO: 1, the CDR2 has the amino acid sequence of SEQ ID NO: 2 and the CDR3 has the amino acid sequence of SEQ ID NO: 3. Preferably, the reduced fucose anti-EGFR antibody additionally comprises a light chain variable region comprising the amino acid sequence of SEQ ID NO: 8 or 10 or an amino acid sequence which is at least 80%, at least 85%, preferably at least 90%, more preferred at least 95% identical thereto, wherein the CDR1 has the amino acid sequence of SEQ ID NO: 4, the CDR2 has the amino acid sequence of SEQ ID NO: 5 and the CDR3 has the amino acid sequence of SEQ ID NO: 6.

According to one embodiment, the reduced fucose anti-EGFR antibody mediates ADCC and is capable of specifically binding to EGFR and blocking dimerization of EGFR, in particular heterodimerization of EGFR with other members of the epidermal growth factor receptor family such as HER2, HER3 and HER4. Further characteristics were described above.

In one embodiment, the reduced fucose anti-EGFR antibody is provided as conjugate comprising the antibody conjugated to a further agent such as a therapeutically active substance. The further agent preferably is useful in therapy and/or monitoring of cancer. For example, the further agent may be selected from the group consisting of radionuclides, chemotherapeutic agents, antibodies, in particular those of different species and/or different specificity than the reduced fucose anti-EGFR antibody, enzymes, interaction domains, detectable labels, toxins, cytolytic components, immunomodulators, immunoeffectors, MHC class I or class II antigens, radioisotopes and liposomes. The further agent, if comprised, may be covalently, in particular by fusion or chemical coupling, or non-covalently attached to the antibody. A particular preferred further agent is a radionuclide or a cytotoxic agent capable of killing cancer cells, such as a chemotherapeutic agent, in particular those described herein elsewhere. Specific examples of chemotherapeutic agents that can be conjugated as further agent include alkylating agents such as cisplatin, anti-metabolites, plant alkaloids and terpenoids, vinca alkaloids, podophyllotoxin, taxanes such as taxol, topoisomerase inhibitors such as irinotecan and topotecan, or antineoplastics such as doxorubicin. The reduced fucose anti-EGFR antibody of the invention may be conjugated to any of the chemotherapeutic agents and/or antibodies described herein. According to one embodiment, which was also used in the examples, the reduced fucose anti-EGFR antibody is not conjugated to a further agent.

### The EGFR Positive Neoplastic Disease and the Patient to be Treated

The reduced fucose anti-EGFR antibodies show unexpectedly high therapeutic efficacy in the patient groups specifically defined herein even when used as single therapeutic agent. Details are described below. Effective treatment of different cancer patients was demonstrated in clinical studies including patients having colon cancer (KRAS mutant and wildtype), lung cancer, gastric cancer, esophageal cancer, renal cell cancer, gallbladder cancer, ovarian cancer, penis cancer and rectum cancer. In particular, a prominent effect was also seen in the treatment of different renal cell carcinomas, including clear cell and non-clear cell renal cell carcinomas.

The EGFR positive neoplastic disease which is to be treated by the reduced fucose anti-EGFR antibody preferably is an EGFR positive cancer was defined and described in detail above and it is referred to the above disclosure. As described, different forms of EGFR positive cancers as well as metastases can be treated with the reduced fucose anti-EGFR antibody according to the invention. The EGFR positive cancer can in particular be selected from the group consisting of malignant epithelial tumors, colon cancer, colorectal cancer, rectum cancer, kidney cancer, ovarian cancer, gastric cancer, esophagus cancer, lung cancer, gallbladder cancer, penis cancer, head and neck cancer, ovarian cancer, breast cancer and uterine cancer. Certain examples of EGFR positive cancer that can be treated are colorectal carcinomas, colon carcinomas, rectum carcinomas, non-small cell lung carcinomas, squamous cell lung cancer, renal cell carcinomas, triple negative breast cancer, squamous cell carcinomas of the head and neck, esophageal adenocarcinomas, gastric adenocarcinomas, gastroesophageal junction adenocarcinomas, endometrical carcinomas or sarcomas and cervical carcinomas, including metastatic forms thereof. Further EGFR positive cancers include renal cell carcinomas such as clear cell renal cell carcinoma, papillary renal cell carcinoma (basophilic and eosinophilic), chromophobe renal cell carcinoma, Bellini duct carcinoma/collecting duct carcinoma, and pleomorpohic (sarcomatoid) carcinoma of the kidney; non small cell lung cancers such as squamous non small cell lung cancer (sNSCLC), and non squamous non small cell lung cancer (nsNSCLC), in particular adenocarcinoma and large cell carcinoma; small cell lung cancer (SCLC); epithelial tumors of the head and neck such as squamous cell cancer of the head and neck (SCCHN), in particular non-differentiated, differentiated, adenoid-squamous and verrucous SCCHN; and gastric cancers such as adenocarcinoma, in particular tubular adenocarcinoma, papillary adenocarcinoma and mucinous adenocarcinoma, signet ring cell carcinoma, adenoid-squamous carcinoma, squamous carcinoma, medullary gastric carcinoma, small cell gastric carcinoma, and non-differentiated gastric carcinoma. The gastric cancer may be located in the pyloric antrum, in the corpus or in the fundus or may be a diffuse gastric cancer in the entire stomach. In certain embodiments, the cancer is a metastasizing cancer. As is shown by the examples, the anti-EGFR antibody of the invention is particularly suitable for treating metastatic cancer and metastases. The EGFR positive cancer may include any type of metastases, such as skin metastases, lymph node metastases, lung metastases, liver metastases, peritoneal metastases, pleural metastases and/or brain metastases. In particular embodiments, the EGFR positive neoplastic disease is metastatic cancer of the large intestine or squamous cell cancer of the head and neck, for which the reduced fucose anti-EGFR antibody according to the present invention is used alone or in combination with radiation therapy and/or other anticancer medicines.

The reduced fucose anti-EGFR antibody of the invention can be used for the treatment of patients that can be treated with conventional anti-EGFR antibodies such as Erbitux®. Especially, the reduced fucose anti-EGFR antibody can be used for treatment of patients having
locally or regionally advanced squamous cell carcinoma of the head and neck in combination with radiation therapy;
recurrent locoregional disease or metastatic squamous cell carcinoma of the head and neck in combination with platinum-based therapy with 5-FU; and/or
recurrent or metastatic squamous cell carcinoma of the head and neck progressing after platinum-based therapy.

Furthermore, the reduced fucose anti-EGFR antibody can be used for treatment of patients
having K-Ras mutation-negative (wild-type), EGFR-expressing, metastatic colorectal cancer (which can be e.g. determined by FDA-approved tests) in combination with FOLFIRI (leucovorin, 5-FU and irinotecan) for first-line treatment;
in combination with irinotecan in patients who are refractory to irinotecan-based chemotherapy; and/or
as a single agent in patients who have failed oxaliplatin- and irinotecan-based chemotherapy or who are intolerant to irinotecan.

Furthermore, as described herein, the special characteristics of the reduced fucose anti-EGFR antibody of the invention provide novel treatment opportunities and thus, allow the treatment of patient groups that can not or can no longer be treated with conventional anti-EGFR antibodies.

Preferably, the EGFR positive neoplastic disease has a detectable EGFR expression, preferably detectable by immunohistochemistry or in-situ hybridization. It especially includes cells having an EGFR expression which is detectable by immunohistochemistry or in-situ hybridization. In particular, an EGFR gene amplification is detectable, preferably by in situ hybridization such as fluorescence in situ hybridization (FISH), silver in situ hybridization (SISH) or chromogen in situ hybridization (CISH). Details were also described above. According to certain embodiments, the EGFR status of the patient is determined prior to treatment.

In certain embodiments, the EGFR positive neoplastic disease includes cells having a mutation in EGFR. Such EGFR mutation may be detectable, preferably by sequence analysis using for example PCR analysis, hybridization analysis or restriction analysis. According to certain embodiments, it is determined prior to treatment whether the EGFR of the patient carries a mutation.

In certain embodiments, the EGFR positive neoplastic disease includes cells having a KRAS mutation, in particular a mutation resulting in constitutively active K-Ras protein. Examples of respective K-Ras mutants are K-Ras having a mutation at amino acid number 12 such as K-Ras G12V, K-Ras G12D, K-Ras G12C, K-Ras G12S, K-Ras G12A and K-Ras G12R; K-Ras having a mutation at amino acid number 13 such as K-Ras G13D and K-Ras G13R; and K-Ras having a mutation at amino acid number 61 such as K-Ras Q61H, K-Ras Q61K, and K-Ras Q61L. As demonstrated in the clinical study and the in vitro experiments, the reduced fucose anti-EGFR antibody according to the invention shows strong activity and good therapeutic results with KRAS mutant tumors and cancer cells. Since the reduced fucose anti-EGFR antibody according to the invention is also effective in KRAS wildtype cancers, it can be used for the treatment of KRAS wt as well as for the treatment of KRAS mutant cancers. Hence, in further embodiments, the EGFR positive neoplastic disease is KRAS wildtype, i.e. it does not comprise cells having a KRAS mutation. In certain embodiments, the anti-EGFR antibody of the invention is for treating an EGFR positive neoplastic disease, wherein the patient to be treated has an unknown KRAS mutation status and/or the reduced fucose anti-EGFR antibody is for treatment of an EGFR positive neoplastic disease irrespective of its KRAS mutation status.

In certain embodiments, the patient to be treated is afflicted with EGFR positive kidney cancer. In certain embodiments, the patient to be treated is afflicted with metastasizing kidney cancer. Kidney cancer includes renal cell carcinoma and urothelial cell carcinoma. In certain embodiments, the reduced fucose anti-EGFR antibody is for the treatment of a renal cell carcinoma. The renal cell carcinoma may be a clear cell renal cell carcinoma, a papillary renal cell carcinoma including basophilic and eosinophilic papillary renal cell carcinoma, a chromophobe renal cell carcinoma, a collecting duct carcinoma or Bellini duct carcinoma, a pleomorpohic and/or sarcomatoid carcinoma or a clear cell papillary renal cell carcinoma. Specific examples of such EGFR positive kidney cancers are clear cell renal cell carcinomas and non-clear cell renal cell carcinomas such as papillary renal cell carcinomas. Patients with kidney cancer generally have a very poor prognosis since usually kidney cancers do not respond to chemotherapy and radiotherapy. Due to this lack of response, kidney cancers, in particular renal cell carcinomas, are known to be the most lethal of all the genitourinary tumors. Metastatic renal cell carcinoma presents a special challenge to oncologists, as about 70% of patients develop metastases during the course of their disease, and 5 year survival for patients with metastatic renal cell carcinoma is between 5 and 15%. Furthermore, there is currently no established adjuvant therapy for renal cell carcinoma after surgical excision of the primary tumor and visible metastases. The use of non-specific cytokines has so far been shown to be ineffective. Unlike most other cancers, renal cell carcinoma is resistant to most cytotoxic and cytostatic agents, which severely limits possible effective adjuvant therapy. Trials of cancer vaccines, radiotherapy, chemotherapy, immunotherapy, or biologic therapies have been met with little success, and currently the standard of care for completely resected high-risk renal cell carcinoma is close observation with no other therapy. The reduced fucose anti-EGFR antibody according to the present invention shows strong therapeutic efficacy against different forms of renal cell carcinomas, in particular clear cell renal cell carcinomas and non-clear cell carcinomas. This is remarkable since previous clinical studies with the high-fucose cetuximab were discouraging and did not showed therapeutic efficacy in renal cell carcinoma patients (see, e.g., Motzer et al. (2003) Investigational New Drugs 21, 99-101). Hence, the present invention provides a new possibility to treat renal cell carcinomas, including clear cell as well as non-clear cell renal cell carcinomas, using the low fucose anti-EGFR antibody according to the invention. These treatment options are in particular important for patients that can not be treated or can not be continued to be treated with other anti-EGFR antibodies, because they have e.g. a mutated KRAS or unknown KRAS status, an unfavorable FcgammaIIIa receptor allotype (158F/F or 158V/F), developed allergic reactions against the other anti-EGFR antibody and/or developed an adverse skin reaction, in particular of grade 3 or higher, during treatment with the other anti-EGFR antibody.

The EGFR positive neoplastic disease can prior to the treatment with the reduced fucose anti-EGFR antibody according to the present invention be resistant to or may have progressed after treatment with one or more anti-cancer agents such as chemotherapeutic agents and/or therapeutic antibodies, in particular one or more of the chemotherapeutic agents described herein and/or after treatment with one or more of the prior art anti-EGFR antibodies described herein. For example, the EGFR positive neoplastic disease may be resistant to or may have progressed after treatment with at least one high fucose anti-EGFR antibody described herein such as cetuximab (Erbitux®) and/or after treatment with one or more anti-EGFR antibodies which cause severe adverse skin reactions of grade 3 or higher in more than 10%, in particular more than 15% or more than 20% of the treated patients. As described in the introduction, also afucosylated antibodies, such as GA201, which are different from the reduced fucose anti-EGFR antibody of the invention, may cause adverse skin reactions of grade 3 or higher. Furthermore, the EGFR positive cancer may be resistant to or may have progressed following radiotherapy.

The patient to be treated may be any human patient suffering from an EGFR positive neoplastic disease such as cancer. In specific embodiments, the patient is a heavily pretreated cancer patient, in particular a patient who was subject to one or more, preferably two or more, or three or more cancer therapies prior to the treatment with the reduced fucose anti-EGFR antibody of the invention. Respective preceding treatments which characterize the patient to be treated are also described below; it is referred to the below disclosure.

As demonstrated by the experimental data, the reduced fucose anti-EGFR antibody of the invention effectively induces ADCC with peripheral blood mononuclear cells (PBMCs) irrespectively of the Fcγ receptor IIIa allotype of the donor. In contrast to high fucose anti-EGFR antibodies which have a much higher ADCC activity in patients being homozygous for valine in amino acid position 158 of the Fcγ receptor IIIa (FcγRIIIa-158V/V) than in other patients, the efficacy of inducing ADCC of the reduced fucose anti-EGFR antibody of the invention is similar for all Fcγ receptor IIIa allotypes. Hence, the patient treated with the reduced fucose anti-EGFR antibody may have any Fcγ receptor IIIa allotype and in particular may be homozygous for valine in amino acid position 158 of the Fcγ receptor IIIa (FcγRIIIa-158V/V), homozygous for phenylalanine in amino acid position 158 of the Fcγ receptor IIIa (FcγRIIIa-158F/F) or heterozygous for valine and phenylalanine in amino acid position 158 of the Fcγ receptor IIIa (FcγRIIIa-158V/F). In preferred embodiments, the reduced fucose anti-EGFR antibody is for treatment of patients irrespective of their FcγRIIIa allotype.

Clinical studies showed that the reduced fucose anti-EGFR antibody according to the invention is well-tolerated by the patients and causes only few adverse reactions. In particular, the frequency and severity of adverse reactions is significantly lower and milder than with common Erbitux® therapy. Especially, adverse skin reactions are a general problem associated with the therapy using common EGFR inhibitors, in particular anti-EGFR antibodies. For example, more than 3 out of 4 patients receiving cetuximab (Erbitux®) therapy suffer from acneiform rash, with up to 17% being of the severe form (grade 3 or higher). Once developed, the rash also does not resolve until after the Erbitux® treatment is terminated or the dosage is reduced. Similar situations are also found for other EGFR inhibitors, including reduced fucose anti-EGFR antibodies described in the art. In the art, the skin rash has even been considered as an indicator for the therapeutic efficacy of the EGFR inhibitor. In contrast thereto, the reduced fucose anti-EGFR antibody according to the invention causes adverse skin reactions much less frequently and only of mild grade, while it nevertheless shows strong therapeutic efficacy. So far, no adverse skin reactions of grade 3 or higher were observed in clinical studies. Therefore, the anti-EGFR antibody of the invention disproves the dogma that a high therapeutic efficacy must be accompanied by strong skin reactions.

Another example of frequent adverse reactions associated with high fucose anti-EGFR antibodies, in particular cetuximab (Erbitux®), is hypomagnesemia. Nearly all patients treated with Erbitux® in clinical monotherapy trials experienced a progressive lowering of the serum magnesium level. In more than 50% of the patients a hypomagnesemia occurred. In clinical studies with the reduced fucose anti-EGFR antibody according to the invention, however, less than 6% of the patients had a decreased blood magnesium level even after several weeks of treatment.

Hence, the reduced fucose anti-EGFR antibody according to the invention is especially useful for treating a human patient with an EGFR positive neoplastic disease, wherein the patient suffers from or is at risk of suffering from an adverse reaction against an EGFR inhibitor or wherein an adverse reaction occurred in a prior treatment with an EGFR inhibitor, in particular with an anti-EGFR antibody. The reduced fucose anti-EGFR antibody according to the invention can in particular be used for treating patients who cannot be or can no longer be treated with a conventional EGFR inhibitor, especially with high fucose anti-EGFR antibodies such as cetuximab (Erbitux®), because of the expected or already occurring adverse reactions caused by said conventional EGFR inhibitor. For example, the patient may be at risk of developing adverse reactions against the conventional EGFR inhibitor, or may be known to develop such adverse reactions in view of previous treatments, or may presently suffer from such adverse reactions due to an ongoing or recently terminated treatment with a conventional EGFR inhibitor such as an anti-EGFR antibody. The adverse reactions in particular are so severe that they outweigh the benefits of the therapy with the conventional EGFR inhibitor. Thus, the anti-EGFR antibody of the invention can in particular be used in therapeutic settings, wherein a treatment with the EGFR inhibitor such as an anti-EGFR antibody causing the adverse reaction cannot be carried out or continued due to said adverse reaction. In such settings, the present invention provides valuable new treatment options. In preferred embodiments, the reduced fucose anti-EGFR antibody according to the invention is for treating a human patient after a treatment with an EGFR inhibitor was terminated or interrupted because of an adverse reaction against said EGFR inhibitor. In particular, the reduced fucose anti-EGFR antibody according to the invention is for treating a human patient who suffered under an adverse reaction, in particular an adverse skin reaction of grade 3 or higher and/or an allergic reaction, against an EGFR inhibitor, in particular an anti-EGFR antibody, in a previous treatment with said EGFR inhibitor. In another embodiment, the reduced fucose anti-EGFR antibody according to the invention is for treating a human patient having an EGFR positive neoplastic disease who is in a poor general health condition and in particular cannot tolerate an adverse reactions such as a skin reaction of grade 3 or higher against an EGFR inhibitor.

Adverse reactions as referred to herein are in particular determined, classified and graded according to the Common Terminology Criteria for Adverse Events of the Cancer Therapy Evaluation Program Version 3.0 of the U.S. National Institute of Health. The grade of an adverse reaction (also called adverse event) refers to its severity with grade 1: mild adverse reaction, grade 2: moderate adverse reaction, grade 3: severe adverse reaction, grade 4: life-threatening or disabling adverse reaction, and grade 5: death related to adverse reaction.

In certain embodiments, the adverse reaction against the EGFR inhibitor, in particular the anti-EGFR antibody, used in the previous treatment is selected from the group consisting of skin reactions such as rash, metabolic reactions such as low serum magnesium level and/or low serum potassium level, and gastrointestinal reactions such as diarrhea, nausea, vomiting and/or constipation. In particular, the adverse reaction against the previously used EGFR inhibitor, which in particular is an anti-EGFR antibody, is an adverse skin reaction, in particular including rash or desquamation such as acneiform rash, erythema multiforme and hand-foot skin reaction; pruritus; itching; nail changes; and/or ulceration. In specific embodiments, the adverse skin reaction caused by the previously used EGFR inhibitor is a severe adverse skin reaction of grade 3 or 4. In certain embodiments, the adverse skin reaction caused by the previously used EGFR inhibitor includes skin rash or acneiform skin rash, in particular severe skin rash or severe acneiform skin rash, especially of grade 3 or grade 4. Skin rash of grade 3 in particular includes severe, generalized erythroderma and/or macular, papular or vesicular eruption; and/or desquamation covering at least 50% of the body skin area. Skin rash of grade 4 in particular includes generalized exfoliative, ulcerative, and/or bullous dermatitis. Acneiform rash of at least grade 3 is in particular associated with pain, disfigurement, ulceration and/or desquamation. In certain embodiments, the adverse skin reaction that occurred during prior treatment with the EGFR inhibitor, in particular an anti-EGFR antibody, is associated with an infection, in particular a bacterial or viral infection, for example an infection of the skin or blood.

Furthermore, the adverse reaction against the previously used EGFR inhibitor can be a metabolic adverse reaction including hypomagnesemia (low serum magnesium levels) and hypokalemia (low serum potassium serum levels). The metabolic adverse reaction occurring during previous treatment can be of grade 2 or higher, in particular grade 3 or grade 4. Hypomagnesemia of grade 2 refers to blood magnesium levels between 1.2 to 0.9 mg/dl or 0.5 to 0.4 mmol/l. Hypomagnesemia of grade 3 refers to blood magnesium levels between 0.9 to 0.7 mg/dl or 0.4 to 0.3 mmol/l. Hypomagnesemia of grade 4 refers to blood magnesium levels lower than 0.7 mg/dl or lower than 0.3 mmol/l. Hypokalemia of grade 3 refers to blood potassium levels between 3.0 to 2.5 mmol/l and hypokalemia of grade 4 refers to blood potassium levels lower than 2.5 mmol/l. In certain embodiments, the adverse reactions against the previously used anti-EGFR antibody include hypomagnesaemia of grade 2 or higher.

The EGFR inhibitor used in the prior treatment causing the adverse reaction is in certain embodiments selected from the group consisting of anti-EGFR antibodies such as Fuc+ cetuximab (Erbitux®), panitumomab (Vectibix), GA201 and tyrosine kinase inhibitors such as gefitinib, erlotinib and lapatinib.

In a further aspect of the present invention, a human patient with an EGFR positive neoplastic disease and an effusion is treated with the reduced fucose anti-EGFR antibody of the invention. Effusion in this respect refers to an irregular third-space fluid collection in the human body, in particular to a fluid collection in a body cavity wherein the fluid is escaped from other body parts. A pleural effusion is a fluid collection in the pleural cavity and a peritoneal effusion or ascites is a fluid collection in the peritoneal cavity. The terms “peritoneal effusion” and “ascites” are used synonymously herein. Cancer patients often suffer from effusions if the cancer also affects the mesothelium, i.e. the membranes which line the different body cavities. Either the primary tumor invades into a mesothelium or metastases colonize a mesothelium and thereby disturb its function in regulating the fluid circulation. For example, involvement of the pleura may result in a pleural effusion while involvement of the peritoneum may lead to peritoneal effusion or ascites. Hence, the effusion as referred to herein in particular is a malignant effusion, i.e. an effusion caused by cancer, in particular a malignant pleural effusion or a malignant peritoneal effusion (malignant ascites).

The clinical data disclosed herein demonstrates that the reduced fucose anti-EGFR antibody according to the present invention shows remarkable efficacy in cancer patients having an effusion, in particular a pleural effusion or a peritoneal effusion. The clinical studies show that the treatment with the reduced fucose anti-EGFR antibody results in a significant reduction of the effusion so that manual puncturing and draining of the effusion was no longer necessary and the effusion ultimately was not detectable any more. This is an important finding since the patients severely suffer under the effusion and the necessary frequent drainage thereof, which are both associated with discomfort and pain and pose the risk of complications. Therefore, the reduced fucose anti-EGFR antibody according to the invention can be used for the treatment of effusions, in particular malignant effusions such as malignant pleural effusions and malignant peritoneal effusions, in patients afflicted with a EGFR positive neoplastic disease, in particular cancer. Hence, the present invention provides a new possibility to treat peritoneal effusions, in particular malignant ascites, using the low fucose anti-EGFR antibody according to the invention. These treatment options are in particular important for patients that can not be treated or can not be continued to be treated with other anti-EGFR antibodies, because they have e.g. a mutated KRAS or unknown KRAS status, an unfavorable FcgammaIIIa receptor allotype (158F/F or 158V/F), developed allergic reactions against the other anti-EGFR antibody and/or developed an adverse reaction, in particular of grade 3 or higher, such as adverse skin reactions, during treatment with the other anti-EGFR antibody. In certain embodiments, the reduced fucose anti-EGFR antibody is for treatment of the EGFR positive neoplastic disease, in particular EGFR positive cancer, and the treatment of an effusion as described above.

The EGFR positive neoplastic disease of the patient with an effusion preferably is selected from the group consisting of pancreatic cancer, ovarian cancer, gastric cancer, esophageal cancer, colon cancer, breast cancer and lung cancer. According to one embodiment, the cancer is a metastasizing cancer. Peritoneal effusion is often associated with pancreatic, ovarian, gastric, esophageal or colon cancer and pleural effusion is often associated with breast, lung, gastric or esophageal cancer. However, the EGFR positive neoplastic disease of the patient with an effusion may also be any other type of cancer as described herein, in particular metastasizing cancer, including kidney cancer such as renal cell carcinoma as described herein. The neoplastic disease in particular affects a mesothelium, especially the pleura and/or the peritoneum. In certain embodiments, the neoplastic disease includes a tumor or metastasis at or in a mesothelium, in particular the pleura and/or the peritoneum. In certain embodiments, the patient with an effusion has gastric and/or esophageal cancer such as a gastric carcinoma or an adenocarcinoma of the esophageal/gastric junction.

The reduced fucose anti-EGFR antibody can be used for treatment as monotherapy. Using the reduced fucose anti-EGFR antibody as monotherapy has the advantage that a therapeutic effect can be achieved while only minor side effects can be expected due to the advantageous properties of the reduced fucose anti-EGFR antibody of the invention. This is an advantage when treating patients at advanced states such as previously treated patients wherein the disease progressed despite treatment and/or patients afflicted with metastatic cancer as this patient group often is in a poor health conditions and thus, is excluded from further aggressive treatment. However, the reduced fucose anti-EGFR antibody according to the present invention can also be used in combination therapy wherein the cancer is additionally treated with one or more anti-cancer therapeutic agents such as chemotherapeutic agents or further anti-cancer antibodies to further improve the therapeutic benefit for the patient. As the reduced fucose anti-EGFR antibody according to the present invention is effective at low dosages and in particular in lower dosages than conventional high fucose anti-EGFR antibodies, such combination therapies provide again novel and useful therapeutic options. In certain embodiments, the reduced fucose anti-EGFR antibody is used in combination with one or more anti-cancer agents such as chemotherapeutic agents and/or one or more further antibodies which are different from the reduced fucose anti-EGFR antibody of the invention. Here, also combination therapies can be used that are established for high fucose anti-EGFR antibodies, in particular cetuximab, such as e.g. a combination treatment with irinotecan. The treatment can also be combined with radiotherapy and/or surgery.

Anti-cancer agents that can be used in combination with the reduced fucose anti-EGFR antibody may be selected from any chemotherapeutic agent, in particular chemotherapeutic agents known to be effective for treatment of EGFR positive cancers. The type of chemotherapeutic agent also depends on the EGFR positive cancer to be treated. Particularly, preferred are combinations with anti-cancer agents that are used for cetuximab (Erbitux®). The combination partner may be selected from the group consisting of taxanes such as paclitaxel (Taxol), docetaxel (Taxotere) and SB-T-1214; cyclophosphamide; lapatinib; erlotinib; imatinib; pazopanib; capecitabine; cytarabine; vinorelbine; gemcitabine; anthracyclines such as daunorubicin, doxorubicin, epirubicin, idarubicin, valrubicin and mitoxantrone; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin), and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues, selective estrogen receptor modulators and estrogen receptor downregulators. If used as combination therapy, the reduced fucose anti-EGFR antibody is preferably used in combination with platinum based chemotherapeutic agents or topoisomerase inhibitors such as irinotecan, in particular with a combination of folinic acid, fluorouracil and oxaliplatin (FOLFOX) or a combination of folinic acid, fluorouracil and irinotecan (FOLFIRI). This particularly, if the reduced fucose anti-EGFR antibody corresponds in its binding behavior and Fv mediated anti-tumor properties essentially to cetuximab. In further preferred embodiments, the reduced fucose anti-EGFR antibody according to the invention is used in combination with one or more chemotherapeutica selected from the group consisting of cisplatin, carboplatin, oxaliplatin, irinotecan, fluorouracil (5-FU) and capecitabine. Here, basically the same combination schedules and administration schemes can be used as are used in the prior art when using a high fucose anti-EGFR antibody, e.g. cetuximab, in combination therapy.

Furthermore, also therapeutic antibodies can be used as combination partner for the reduced fucose anti-EGFR antibody. It may be any antibody that is useful in cancer therapy which is different from the reduced fucose anti-EGFR antibody. In particular, the further antibody is approved for cancer treatment by an administration such as the U.S. Food and Drug Administration (FDA), the European Medicines Agency (EMA, formerly EMEA) and the Bundesinstitut für Arzneimittel and Medizinprodukte (BfArM). Examples of the further antibody that can be used for combination treatment with the reduced fucose anti-EGFR antibody are anti-EGFR antibodies such as panitumomab (Vectibix) and nimotuzumab (Theraloc) (which is particularly feasible if the reduced fucose anti-EGFR antibody shows cross-specificity with cetuximab and preferably is a reduced fucose cetuximab antibody), anti-HER2 antibodies such as trastuzumab (Herceptin) and pertuzumab; anti-VEGF antibodies such as bevacizumab (Avastin); anti-CD52 antibodies such as alemtuzumab (Campath); anti-CD30 antibodies such as brentuximab (Adcetris); anti-CD33 antibodies such as gemtuzumab (Mylotarg); and anti-CD20 antibodies such as rituximab (Rituxan, Mabthera), tositumomab (Bexxar) and ibritumomab (Zevalin). In certain embodiments, the reduced fucose anti-EGFR antibody is used in combination with bevacizumab. According to one embodiment, the reduced fucose antibody of the invention is not used in combination with an anti-EGFR antibody which is known to cause adverse skin reactions of grade 3 or higher, such as Erbitux®, panitumumab or GA201. According to one embodiment, the reduced fucose anti-EGFR antibody is not used in combination with another EGFR inhibitor.

As shown in the examples, the reduced fucose anti-EGFR antibody according to the present invention is especially effective in the treatment of renal cell carcinomas. Hence, in certain embodiments the reduced fucose anti-EGFR antibody does not need to be and in certain embodiments is not combined with a further agent which is therapeutically active against renal cell carcinomas.

The reduced fucose anti-EGFR antibody provided herein preferably is for treatment of an EGFR positive primary tumor, an EGFR positive recurrent tumor and/or EGFR positive metastases of such tumors, and in particular is useful for treatment before, during or after surgery and for the prevention or treatment of metastases. As is demonstrated by the present invention, the treatments with the reduced fucose anti-EGFR antibody described herein are also particularly useful for the treatment, including prevention, of effusions, in particular pleural and peritoneal effusions.

The reduced fucose anti-EGFR antibody in particular is for the treatment of a patient as adjuvant therapy. In certain embodiments, the reduced fucose anti-EGFR antibody is for the treatment of a patient as neoadjuvant therapy or in a combined neoadjuvant-adjuvant therapy. Furthermore, the reduced fucose anti-EGFR antibody is for the treatment of a patient as palliative therapy.

As is shown by the examples, the treatment with the reduced fucose anti-EGFR antibody as taught herein is therapeutically successful and in particular can result in tumor or metastases remission or a stabilization of the disease. In particular, in the analyzed patients significant stabilizations of the disease were observed, what are important successes in particular for the group of heavily pretreated patients which basically have no or only limited further therapeutic options. In particular, the examples show that the treatment with the reduced fucose anti-EGFR antibody described herein may result in the inhibition of tumor growth, the reduction of tumor size, the prevention of further metastases (either of the same or of a different type) and/or the reduction of the number or size of metastases. Further beneficial results of the treatment include a reduction of lesions caused by the tumor and/or metastases and/or a reduction of effusions, in particular pleural or abdominal effusions. Furthermore, significant reduction of important tumor markers such as CEA and CA19-9 were observed. This also is an important indicator for the therapeutic effect. Due to the therapeutic effects obtained with the treatment of the present invention, an increase in progression-free survival and/or lifespan as well as an improvement of the general health condition of the patients can be achieved.

In certain embodiments, the reduced fucose anti-EGFR antibody according to the invention is for the treatment of a primary or recurrent tumor and/or the treatment of a metastasis, including peritoneal metastases, pleural metastases, lung metastases and/or liver metastases. In certain embodiments, the reduced fucose anti-EGFR antibody is for the treatment of a lesion caused by a tumor or metastasis, in particular a lesion of a mesothelium, in particular the pleura and/or the peritoneum. Furthermore, the reduced fucose anti-EGFR antibody may be used in the treatment of effusion, in particular pleural effusion and/or peritoneal effusion such as ascites.

The treatment with the reduced fucose anti-EGFR antibody preferably results in inhibition of tumor growth and in particular reduction of tumor size. Furthermore, the occurrence of further metastases is prevented and/or their number is reduced by the treatment. In particular, the treatment with the reduced fucose anti-EGFR antibody results in reduction of lesions caused by a tumor and/or one or more metastases. In certain embodiments, the treatment leads to a reduction in tumor markers, preferably in the level of the tumor markers CEA and/or CA19-9. The treatment with the reduced fucose anti-EGFR antibody also may lead to a reduction of effusion volume, in particular of a pleural and/or abdominal effusion. The treatment preferably results in an increase in progression-free survival; and/or an increase in lifespan and thus the overall survival.

As demonstrated herein, the reduced fucose anti-EGFR antibody according to the invention exerts its therapeutic activity by several different mechanisms of action. These include the direct inhibition of EGFR on the tumor cells, the induction of ADCC, the induction of granulocyte-based immune responses and the induction of macrophage-based immune responses. In preferred embodiments, the treatment of the EGFR positive neoplastic disease with the reduced fucose anti-EGFR antibody according to the present invention involves one or more, two or more or preferably all of the following:
(i) inhibiting EGFR activation on cells of the EGFR positive neoplastic disease, in particular via binding of said EGFR by the reduced fucose anti-EGFR antibody;
(ii) inducing antibody-dependent cellular cytotoxicity directed against cells of the EGFR positive neoplastic disease, in particular via activating immune cells, preferably natural killer cells, by the reduced fucose anti-EGFR antibody bound to said cells of the EGFR positive neoplastic disease;
(iii) inducing granulocytes, in particular neutrophil and eosinophil granulocytes, to attack and preferably destroy cells of the EGFR positive neoplastic disease, in particular via binding and activation of granulocytes by the reduced fucose anti-EGFR antibody bound to said cells of the EGFR positive neoplastic disease; and/or
(iv) inducing macrophages to attack and preferably destroy cells of the EGFR positive neoplastic disease, in particular via activation of macrophages by the reduced fucose anti-EGFR antibody bound to said cells of the EGFR positive neoplastic disease.

In preferred embodiments, the treatment of the EGFR positive neoplastic disease with the reduced fucose anti-EGFR antibody involves binding and activation of granulocytes, in particular neutrophil and eosinophil granulocytes, by the reduced fucose anti-EGFR antibody that is bound to cells of the EGFR positive neoplastic disease; and inducing said activated granulocytes to destroy said cells of the EGFR positive neoplastic disease. Furthermore, the treatment of the EGFR positive neoplastic disease with the reduced fucose anti-EGFR antibody preferably involves activation of macrophages by the reduced fucose anti-EGFR antibody that is bound to cells of the EGFR positive neoplastic disease; and inducing said activated macrophages to destroy said cells of the EGFR positive neoplastic disease.

In a specific aspect, the present invention provides an anti-EGFR antibody with a glycosylation site in the CH2 domain, wherein 50% or less of the glycans attached to said glycosylation site carry fucose (reduced fucose anti-EGFR antibody) and wherein the reduced fucose anti-EGFR antibody is capable of inducing an antibody-dependent cellular cytotoxicity reaction, for inducing a granulocyte-driven immune reaction against cells of an EGFR positive neoplastic disease in a human patient. The granulocyte-driven immune reaction preferably involves binding and activation of granulocytes by the reduced fucose anti-EGFR antibody bound to said cells of the EGFR positive neoplastic disease; and inducing said activated granulocytes to destroy said cells of the EGFR positive neoplastic disease. The granulocytes may for example be neutrophil granulocytes and/or eosinophil granulocytes.

In a further aspect, the present invention provides an anti-EGFR antibody with a glycosylation site in the CH2 domain, wherein 50% or less of the glycans attached to said glycosylation site carry fucose (reduced fucose anti-EGFR antibody) and wherein the reduced fucose anti-EGFR antibody is capable of inducing an antibody-dependent cellular cytotoxicity reaction, for inducing a macrophage-driven immune reaction against cells of an EGFR positive neoplastic disease in a human patient. The macrophage-driven immune reaction preferably involves activation of macrophages by the reduced fucose anti-EGFR antibody bound to said cells of the EGFR positive neoplastic disease; and inducing said activated macrophages to destroy said cells of the EGFR positive neoplastic disease.

In certain embodiments, the treatment with the reduced fucose anti-EGFR antibody includes the induction of a cytokine release after administration of the first dose of the reduced fucose anti-EGFR antibody. The cytokine may be selected from the group consisting of IFN-γ, IL-6, IL-8, TNF-α, IP-10 and IL-1ra. Preferably all of these cytokines are released. The cytokines are in particular released into the blood stream of the patient and are detectable in the patient's blood. One or more, preferably all of these cytokines are in particular released in at least 10%, preferably at least 20%, at least 30%, at least 40% or at least 50% of the patients treated. Preferably, the release of the cytokine results in a peak increase of the concentration of said cytokine in the patient's blood of at least 2-fold, preferably at least 5-fold or at least 10-fold.

In certain embodiments, the reduced fucose anti-EGFR antibody according to the invention is for treating an EGFR positive neoplastic disease in a human patient, wherein treatment conditions are used which for at least one other anti-EGFR antibody cause an adverse skin reaction in at least 50%, in particular at least 60% or at least 70%, of the patients when using said other anti-EGFR antibody. In particular, the at least one other anti-EGFR antibody is selected from the group consisting of high fucose cetuximab (Erbitux®), panitumumab, zalutumumab and GA201. The treatment with the reduced fucose anti-EGFR antibody of the invention preferably causes adverse skin reactions (which include any grade) in not more than 45%, preferably not more than 40%, more preferably not more than 35% of the treated patients. The adverse skin reaction in particular is skin rash and/or acneiform rash. Details were described above.

In further embodiments, the reduced fucose anti-EGFR antibody according to the invention is for treating a neoplastic disease in a human patient, wherein treatment conditions are used which for at least one other anti-EGFR antibody cause an adverse skin reaction of grade 3 or higher in at least 12%, in particular at least 15% or at least 17%, of the patients when using said other anti-EGFR antibody. In particular, the at least one other anti-EGFR antibody is selected from the group consisting of high fucose cetuximab (Erbitux®), panitumumab, zalutumumab and GA201. The treatment with the reduced fucose anti-EGFR antibody of the invention preferably causes adverse skin reactions of grade 3 or higher in not more than 10%, not more than 8%, preferably not more than 6%, more preferably not more than 5% of the treated patients. The adverse skin reaction in particular is skin rash and/or acneiform rash.

The treatment conditions in particular include the dosage regimen including the amount of antibody administered per dose, the dosing interval and/or the type of administration; and/or the condition of the treated patient. Preferably, the percentage incidence of the adverse skin reaction is determined in a clinical study. The percentage incidence of the adverse skin reaction is in particular determined under similar or the same conditions for the reduced fucose anti-EGFR antibody according to the invention and the other anti-EGFR antibody, preferably including similar, overlapping or the same dosage ranges, similar or the same administration intervals and similar patient groups with respect to age, tumor grade and tumor type.

According to certain embodiments, the reduced fucose anti-EGFR antibody of the invention is for treating a human patient that is afflicted with an EGFR positive neoplastic disease for which disease it has been shown that at least one other anti-EGFR antibody shows adverse skin reactions in more than 50%, more than 60% or more than 70% of the treated patients. Examples of such EGFR positive neoplastic diseases and preferred embodiments are described herein and it is referred to the respective disclosure.

### The Preceding Treatments

The present inventors found that the reduced fucose anti-EGFR antibody according to the present invention shows high therapeutic efficacy and clinical success even in patients which failed multiple prior anti-cancer treatments, in particular pretreatments with chemotherapeutic agents and/or other anti-cancer antibodies, in particular anti-EGFR antibodies such as high fucose anti-EGFR antibodies (e.g. Erbitux®). The observed effects are remarkable as a cancer therapy is more prone to failure the further the disease has progressed and in particular if metastasis has progressed. After multiple treatments, the cancer cells are often highly mutated and thereby more easily evade treatment. Furthermore, the tumor load, i.e. the number of tumor cells in the patient, increases with progression of the disease. At higher tumor cell numbers, the killing of some tumor cells may be outweighed by the proliferation of the remaining tumor cells. The same applies to the development of metastases. Hence, the shown therapeutic effects of the reduced fucose anti-EGFR antibody in heavily pretreated patients and in particular in patients with wide spread metastases is impressive and unexpected and also provide novel treatment options for novel patient groups.

In view of these findings, the reduced fucose anti-EGFR antibody according to the invention is particularly for treatment of an EGFR positive neoplastic disease, in particular EGFR positive cancer, in a patient who has received one or more previous treatments of said EGFR positive neoplastic disease. The preceding treatments of the neoplastic disease include treatments with one or more chemotherapeutic agents, radiation treatments (radiotherapy), treatments with one or more therapeutic antibodies which are different from the reduced fucose anti-EGFR antibody, in particular treatments with one or more high fucose anti-EGFR antibodies which antibody and combinations of two or more of these treatments. In certain embodiments, the preceding treatments include at least one treatment with an EGFR inhibitor such as an anti-EGFR antibody, wherein the patient suffered from an adverse skin reaction caused by said EGFR inhibitor. In specific embodiments, the preceding treatment with the EGFR inhibitor had to be interrupted or discontinued or the dose of the EGFR inhibitor had to be reduced because of the occurrence of said adverse skin reaction, in particular because of the occurrence of a grade 3 or 4 adverse skin reaction. The adverse skin reaction that occurred during prior treatment with the EGFR inhibitor such as an anti-EGFR antibody, may in particular include skin rash and/or acneiform skin rash as described herein and/or may be a severe adverse skin reaction, in particular of grade 3 or higher, as described herein.

Furthermore, the EGFR positive neoplastic disease may have been treated by surgery prior to the treatment with the reduced fucose anti-EGFR antibody. According to one embodiment, the preceding treatment of the patient involved cancer surgery, preferably a surgical removal of at least a part of the primary tumor and/or of metastases.

In preferred embodiments, the patient was subject to two or more, preferably three or more preceding anti-cancer treatments prior to the treatment with the reduced fucose anti-EGFR antibody. The preceding treatments may comprise at least one treatment with a high fucose anti-EGFR antibody such as in particular cetuximab either as monotherapy or in combination with a further therapy such as one or more chemotherapeutic agents and/or radiotherapy and/or one or more further antibodies which are directed against an antigen different from EGFR. In particular embodiments, the patient has been treated with at least two, preferably at least three or at least four different anti-cancer agents such as chemotherapeutic agents and/or therapeutic antibodies prior to the treatment with the reduced fucose anti-EGFR antibody. One or more, in particular all of the preceding treatments have failed and the EGFR positive cancer reoccurred or progressed following the preceding treatments.

In certain embodiments, however, the reduced fucose anti-EGFR antibody according to the present invention is used for the treatment of an EGFR positive neoplastic disease which was not previously treated with a chemotherapeutic agent or an anti-cancer antibody. The reduced fucose anti-EGFR antibody hence can be used as first-line therapy.

### High Fucose Anti-EGFR Antibody Used in the Preceding Treatment

In certain aspects and embodiments of the invention, the reduced fucose anti-EGFR antibody is used after a treatment with an EGFR inhibitor, in particular a high fucose anti-EGFR antibody, was discontinued because of an adverse reaction such as an adverse skin reaction as described herein caused by said EGFR inhibitor, and/or after the failed treatment of the patient with a high fucose anti-EGFR antibody used in the prior treatment. Preferably, the reduced fucose anti-EGFR antibody and the high fucose anti-EGFR are based on the same antibody and thus in particular bind the same antigen and comprise the same CDR regions but differ from each other in their glycosylation in the Fc region, in particular in their amount of fucose. The reduced fucose anti-EGFR antibody has a lower amount of fucose than the high fucose anti-EGFR antibody and is capable of mediating a stronger ADCC response. Furthermore, it preferably has a higher amount of bisGlcNAc as described above.

The high fucose anti-EGFR antibody used in the prior treatment preferably has an amount of fucose in its CH2 domain which is 60% or more, 65% or more, 70% or more, or 75% or more. Respective high fucose antibodies are obtained when producing the antibody in standard cell lines such as CHO cells or SP2/0 cells. E.g. the antibody cetuximab (Erbitux®) which is produced in SP2/0 cells is a high fucose anti-EGFR antibody with more than 70% fucose in the carbohydrate chain that is attached to the CH2 domain. In preferred embodiments, the amount of fucose in the CH2 domain of the reduced fucose anti-EGFR antibody is at least 20 percentage points, preferably at least 30 percent points, more preferably at least 40 percentage points, at least 45 percentage points or at least 50 percentage points, or even at least 55 percentage points lower than the amount of fucose in the CH2 domain of the high fucose anti-EGFR antibody. E.g. if the high fucose anti-EGFR antibody has a fucose content of 70% and the reduced fucose anti-EGFR antibody has a fucose content that is 50 percentage points lower, it has a fucose content of 20%. According to one embodiment, the reduced fucose anti-EGFR antibody is afucosylated and does not comprise fucose.

In further embodiments, the high fucose anti-EGFR antibody used in the prior treatment has an amount of bisGlcNAc in the CH2 domain of 10% or less, 7% or less or 5% or less, more preferably 4% or less or does not comprise bisGlcNAc. The amount of bisGlcNAc in the CH2 domain of the reduced fucose anti-EGFR antibody preferably is at least 5 percentage points, more preferably at least 7 percentage points, most preferably at least 10 percentage points higher than the amount of bisGlcNAc in the CH2 domain of the high fucose anti-EGFR antibody. Furthermore, the high fucose anti-EGFR antibody may comprise an amount of galactose of 80% or less, 70% or less or 65% or less, in particular 60% or less. The amount of galactose of the reduced fucose anti-EGFR antibody preferably is at least 10 percentage points higher, more preferably at least 15 percentage points higher or at least 20 percentage points higher, most preferably at least 25 percentage points higher than the amount of galactose of the high fucose anti-EGFR antibody. In certain embodiments, the high fucose anti-EGFR antibody comprises an amount of glycans carrying two galactose units of 40% or less, 35% or less or 30% or less, in particular 25% or less. The amount of glycans carrying two galactose units of the reduced fucose anti-EGFR antibody preferably is at least 15 percentage points higher, more preferably at least 10 percentage points higher or at least 15 percentage points higher, most preferably at least 20 percentage points higher than the amount of glycans carrying two galactose units of the high fucose anti-EGFR antibody.

The high fucose anti-EGFR antibody preferably is of the same antibody type as the reduced fucose anti-EGFR antibody, and in particular is an IgG antibody, preferably an IgG1 antibody. Preferably, the high fucose anti-EGFR antibody is capable of specifically binding to the same epitope as the reduced fucose anti-EGFR antibody of the invention and/or shows cross-specificity with the reduced fucose anti-EGFR antibody. In certain embodiments, the high fucose anti-EGFR antibody has heavy chain and/or light chain amino acid sequences which are at least 80%, at least 90% or at least 95%, more preferably 100% identical to the corresponding amino acid sequences of the reduced fucose anti-EGFR antibody. In particular, the amino acid sequences of the heavy chain CDRs and/or the light chain CDRs are identical to the corresponding amino acid sequences of the CDRs of the reduced fucose anti-EGFR antibody. In preferred embodiments, the high fucose anti-EGFR antibody that was used in the pretreatment is the antibody cetuximab (Erbitux®) or shows cross-specificity with the antibody cetuximab.

According to one embodiment, the high fucose anti-EGFR antibody that was used in the pretreatment is capable of blocking ligand binding and/or dimerization of EGFR, in particular heterodimerization of EGFR with other members of the epidermal growth factor receptor family such as HER2, HER3 and HER4.

According to one embodiment, the high fucose anti-EGFR antibody that was used in the pretreatment specifically binds to an epitope of EGFR which is different from the epitope of the reduced fucose anti-EGFR antibody. In this embodiment, the reduced fucose anti-EGFR antibody and the high fucose anti-EGFR antibody have different CDR sequences. In certain embodiments, the high fucose anti-EGFR antibody is the antibody panitumumab (Vectibix) or shows cross-specificity with the antibody panitumumab.

The high fucose anti-EGFR antibody may be a complete antibody or a fragment or derivative of an antibody. The high fucose anti-EGFR antibody used in the pretreatment may be conjugated to a further therapeutic agent. Examples of suitable therapeutic agent are radionuclides and chemotherapeutic agents, in particular chemotherapeutic agents as described herein, for example maytansine. According to one embodiment, the high fucose anti-EGFR antibody used in the previous treatment is no conjugate. The preceding treatment with the high fucose anti-EGFR antibody may be a monotherapy or a combination therapy together with one or more chemotherapeutic agents and/or one or more further antibodies and/or radiotherapy. Suitable chemotherapeutic agents and further antibodies are those described herein elsewhere.

As is shown in the examples, the reduced fucose anti-EGFR antibody used according to the present invention has a higher therapeutic efficacy than the corresponding high fucose anti-EGFR antibody. It was also observed that the therapeutic efficacy of the reduced fucose anti-EGFR antibody is still higher than that of a corresponding high fucose anti-EGFR antibody even when the reduced fucose anti-EGFR antibody is administered at the same dose but less frequently than the high fucose anti-EGFR antibody and/or when the reduced fucose anti-EGFR antibody is administered at the same frequency but at a lower dose than the high fucose anti-EGFR antibody. Therefore, advantageously, the dosages can be lowered and treatment cycles can be prolonged when using the reduced fucose anti-EGFR antibody according to the invention. Furthermore, significantly less and milder adverse skin reactions were observed in the clinical studies which is a further important advantage. As the adverse skin reactions observed with prior art anti-EGFR antibodies is dose related, the advantageous profile of the anti-EGFR antibody of the invention also allows to increase the dosage if desired. Therefore, the usable dosage range is broadened when using the anti-EGFR antibody of the invention which again is important for therapy.

### Further Antibodies Used in the Preceding Treatment

According to one embodiment, the human patient has been previously treated with an anti-EGFR antibody, and wherein said previous treatment was interrupted, terminated or wherein the dosage of the anti-EGFR antibody had to be reduced because an adverse skin reaction, in particular of grade 3 or higher, occurred during treatment. According to one embodiment, the human patient has been previously treated with an anti-EGFR antibody, and wherein said previous treatment with the anti-EGFR antibody, is not or cannot be continued because an adverse skin reaction, in particular of grade 3 or 4, against said anti-EGFR antibody occurred. Anti-EGFR antibodies known to cause such severe skin reactions in a significant number of patients include but are not limited to Erbitux®, GA201, panitumumab and zalutumumab.

According to one embodiment, at least one therapeutic antibody was used in the preceding treatment which is different from the reduced fucose anti-EGFR antibody. Respective antibodies may also include antibodies which are directed against other antigens and/or do not specifically bind EGFR. These further antibodies that could have been used in the pretreatment preferably specifically bind antigens which are present on tumor cells and which preferably are not present on non-tumor cells or are present on non-tumor cells in a lower amount or at sites which are not accessible for the antibodies. Preferably, the further antibodies are approved for cancer treatment by an administration such as the U.S. Food and Drug Administration (FDA), the European Medicines Agency (EMA, formerly EMEA) and the Bundesinstitut für Arzneimittel and Medizinprodukte (BfArM). Preferred examples of the further antibody are anti-HER2 antibodies such as trastuzumab (Herceptin) and pertuzumab (Omnitarg); anti-VEGF antibodies such as bevacizumab (Avastin); anti-CD52 antibodies such as alemtuzumab (Campath); anti-CD30 antibodies such as brentuximab (Adcetris); anti-CD33 antibodies such as gemtuzumab (Mylotarg); and anti-CD20 antibodies such as rituximab (Rituxan, Mabthera), tositumomab (Bexxar) and ibritumomab (Zevalin).

The further antibody may be a complete antibody or a fragment or derivative of an antibody. In one embodiment, the further antibody is conjugated to a further therapeutic agent. Examples of such therapeutic agents are radionuclides and chemotherapeutic agents, in particular chemotherapeutic agents as described herein. According to one embodiment, the further antibody that was used in the preceding treatment is no conjugate.

### The Chemotherapeutic Agents Used in the Preceding Treatment

In certain embodiments, the preceding treatments include one or more treatments with a chemotherapeutic agent or with a combination of two or more chemotherapeutic agents, optionally in combination with one or more therapeutic antibodies different from the reduced fucose anti-EGFR antibody. The chemotherapeutic agents may be any chemotherapeutic agents and may be selected from the group consisting of cyclophosphamide; lapatinib; capecitabine; cytarabine; vinorelbine; gemcitabine; maytansine; anthracyclines such as daunorubicin, doxorubicin, epirubicin, idarubicin, valrubicin and mitoxantrone; taxanes such as paclitaxel (Taxol), docetaxel (Taxotere) and SB-T-1214; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin); alkylating agents such as dacarbazine; and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues. In particular, the preceding treatment included one or more treatments with a combination of different chemotherapeutic agents such as a combination of folinic acid and fluorouracil and oxaliplatin (FOLFOX), or a combination of folinic acid and fluorouracil and irinotecan (FOLFIRI). In certain embodiments, these combinations of chemotherapeutic agents are further combined with one or more anti-cancer antibodies as described above, in particular high fucose anti-EGFR antibodies such as cetuximab (Erbitux®) or anti-VEGF antibodies such as bevacizumab (Avastin).

### The Composition Comprising the Reduced Fucose Anti-EGFR Antibody and Dosages

The reduced fucose anti-EGFR antibody can be comprised in a pharmaceutical composition. Preferably said pharmaceutical composition is suitable for intravenous injection. It may be an aqueous solution comprising the antibody, or a composition which can be used to prepare a composition suitable for intravenous injection, for example a lyophilized antibody composition. The composition comprising the reduced fucose anti-EGFR antibody may additionally comprise one or more further components selected from the group consisting of solvents, diluents, and excipients. The components of the composition preferably are all pharmaceutically acceptable. The composition may be a solid or fluid composition, in particular a—preferably aqueous—solution, emulsion or suspension or a lyophilized powder. Formulations for preparing antibodies as pharmaceutical compositions are well-known in the prior art and thus, do not need any detailed description.

The composition preferably comprising the reduced fucose anti-EGFR antibody in a concentration in the range of from 1 mg/ml to 100 mg/ml, more preferably from 2 mg/ml to 50 mg/ml, from 2.5 mg/ml to 30 mg/ml or from 3 mg/ml to 25 mg/ml, in particular about 5 mg/ml or about 15 mg/ml.

The reduced fucose anti-EGFR antibody may be administered to the patient by any suitable administration route, preferably by intravenous injection. In preferred embodiments, the reduced fucose anti-EGFR antibody is administered in a dose in the range of from 0.5 to 50 mg, 2 to 40 mg, 4 to 30 mg, preferably 6 to 25 mg, more preferably 8 to 20 mg per kg body weight of the patient. Here, it was found that the reduced fucose anti-EGFR antibodies can be administered at low dosages which still elicit a therapeutic effect even when given as monotherapy. Therefore, advantageously lower dosages can be used. However, due to the improved therapeutic profile and in particular the significantly reduced and only mild incidences of adverse reactions such as in particular adverse skin reactions, also high dosages can be used. Thus, also dosages that are the same or even higher than those commonly used for anti-EGFR antibodies such as Erbitux® are suitable. Therefore, the applicable dosage range is significantly broadened. In particular, the reduced fucose anti-EGFR antibodies is for administration in an amount of 750 mg or less per week, preferably 700 mg or less per week, 600 mg or less per week, 500 mg or less per week, 400 mg or less per week, 650 mg or less per week, more preferably 300 mg or less per week, 250 mg or less per week, 200 mg or less per week, 175 mg or less per week, most preferably 150 mg or less per week. In certain embodiments, the reduced fucose anti-EGFR antibody is administered in a dose per administration in the range of from 10 mg to 2500 mg, 50 mg to 2250 mg, preferably from 100 mg to 2000 mg, 150 mg to 1900 mg, 175 mg to 1800 mg, 200 mg to 1750 mg, 225 mg to 1700 mg, 250 mg to 1600 mg, 275 mg to 1500 mg, 300 mg to 1400 mg, 325 mg to 1300 mg, from 350 mg to 1200 mg, from 375 mg to 1100 mg, from 400 mg to 1000 mg, from 425 mg to 950 mg, 450 mg to 900 mg, 475 mg to 850 mg and 500 mg to 800 mg. Due to the low incidence of adverse skin reactions and other adverse reactions induced by the reduced fucose anti-EGFR antibody according to the invention, the reduced fucose anti-EGFR antibody may also be administered at higher doses, in particular at a dose in the range of from 500 mg to 2000 mg, preferably from 600 mg to 1500 mg or from 700 mg to 1400 mg, especially at doses of 800 mg or more, preferably 900 mg or more or 1000 mg or more. Sometimes, the dose is indicated as mg/m² body surface instead of an absolute dosage. In this case, the above mentioned absolute dosages per administration are divided in half to indicate the dosage/m² as the human body surface area is roughly about 2 m².

Preferably, the reduced fucose anti-EGFR antibody is administered in intervals in the range of from 1 day to 4 weeks, preferably from 2 days to 3 weeks, more preferably from 3 days to 2 weeks or from 5 days to 9 days, and in particular is administered every week or every second week. According to one embodiment, the treatment comprises administering to the patient the reduced fucose anti-EGFR antibody in an initial dose which is higher than the subsequent doses. For example, the initial dose comprises 1.1 to 10 times the normal dose, e.g. as described above, preferably 1.2 to 2 times the normal dose or 1.3 to 1.6 times the normal dose. The term “normal dose” in this respect refers to the subsequent doses which are administered after the initial dose. Preferably, the reduced fucose anti-EGFR antibody is used in long term treatment. In preferred embodiments, the treatment of the patient with the reduced fucose anti-EGFR antibody encompasses at least 4, preferably at least 5, at least 6, at least 7, at least 8, at least 10, at least 12, at least 15, at least 20 or at least 25 administrations of the reduced fucose anti-EGFR antibody. The patient in particular is treated with the reduced fucose anti-EGFR antibody for at least 4 weeks, preferably at least 5 weeks, at least 6 weeks, at least 7 weeks, at least 8 weeks, at least 10 weeks, at least 3 months, at least 4 months, at least 6 months, at least 9 months or at least 1 year. The therapy is thus intended for long-term therapy.

In specific embodiments, the reduced fucose anti-EGFR antibody is administered in one week intervals with an initial dose in the range of from 800 mg to 1200 mg, in particular from 950 mg to 1050 mg such as about 990 mg, and with subsequent doses in the range of from 500 mg to 950 mg, in particular from 650 mg to 800 mg such as about 720 mg. In certain embodiments, this dosage ranges may also be used for administration every second week. In another embodiment, the reduced fucose anti-EGFR antibody is administered in intervals of two weeks with each dose being in the range of from 1100 mg to 1700 mg, preferably from 1300 mg to 1450 mg, in particular about 1370 mg.

The administration of antibodies by injection, including infusion, may cause adverse reactions in the patient's body, in particular infusion related reactions (IRR). Respective effects can also occur when administering the reduced fucose anti-EGFR antibody. To reduce respective infusion related reactions, the treatment of the reduced fucose anti-EGFR antibody may be combined with measures and/or means for treatment, reduction or prevention of such infusion related reactions.

According to one embodiment of the invention, the prevention or reduction of IRR is achieved by combining the treatment of the reduced fucose anti-EGFR antibody with a pre-medication of an agent with analgesic and/or antipyretic properties. Said agent may have one or more of the following characteristics: it is a non-opioid analgesic, it is a non-salicylate analgesic, it is an aniline analgesic/aniline derivative, it is an acetanilide derivative, it is an aminophenol derivative, it is an acetylaminophenol, it is a cyclo-oxygenase inhibitor and/or it is prostaglandin inhibitor. Preferably N-(4-hydroxyphenyl)acetamide (paracetamol or acetaminophen) is used as analgesic and/or antipyretic agent. The agent e.g. is administered intravenously or orally.

It was found by the inventors that such a pre-medication significantly reduces IRR associated with the administration of the reduced fucose anti-EGFR antibody. Hence in one aspect of the invention this pre-medication is use to prevent or treat IRR caused by the administration of reduced fucose anti-EGFR antibody. Exemplary infusion related reactions are fever, edema such as angioedema, arthralgia and shivering.

The agent with analgesic and/or antipyretic properties preferably is administered in a dose from 250 mg to 1500 mg, at least 500 mg, preferably at least 700 mg, at least 800 mg, at least 900 mg, more preferably of 1000 mg. It is preferably administered prior to administration of the reduced fucose anti-EGFR antibody, preferably in one single dose or in two or more, preferably two separate doses.

In preferred embodiments, the agent is administered 5 min to 6 h, preferably 10 min to 4 h, 15 min to 3 h or 20 min to 2 h, more preferably 30 min to 90 min, in particular 1 hour before administration of the reduced fucose anti-EGFR antibody, in particular as a single dose.

In certain preferred embodiments the agent is administered in two doses, whereas a first dose is administered at 8 h to 48 h, preferably 12 h to 36 h or 16 h to 24 h, in particular at the evening before (i.e. about 12 hours before) administration of the reduced fucose anti-EGFR antibody. The second dose is administered 5 min to 6 h, preferably 10 min to 4 h, 15 min to 3 h or 20 min to 2 h, more preferably 30 min to 90 min, in particular 1 hour before administration of the reduced fucose anti-EGFR antibody. In a particular preferred embodiment a first dose of the agent is administered the evening before the administration of the antibody and a second dose is given 1 hour before the administration of the antibody. Preferably both doses are 1000 mg of the agent. A particular preferred agent of this administration scheme is N-(4-hydroxyphenyl)acetamide.

In preferred embodiments, the agent with analgesic and/or antipyretic properties is administered only before the first administration of the reduced fucose anti-EGFR antibody and optionally additionally before any administration of the reduced fucose anti-EGFR antibody after infusion related reactions were induced by the previous administration of the reduced fucose anti-EGFR antibody. In further embodiments, the agent is administered upon occurrence of infusion related reaction to the administration of the reduced fucose anti-EGFR antibody. By restricting the number of premedications against IRR, a potential negative effect of said premedication on the efficacy of the ADCC induced by the reduced fucose anti-EGFR antibody can be minimized.

The agent with analgesic and/or antipyretic properties may be administered in combination with one or more steroids, preferably glucocorticoids, such as cortisol, cortison acetate, cloprenol, prednisone, prednisolone, deflazacort, fluocortolon, triamcinolone, betamethasone or dexamethasone, in particular methylprednisolone. The steroid preferably is administered 5 min to 4 h, more preferably, 15 min to 1 h, most preferably about 30 min before administration of the reduced fucose anti-EGFR antibody. The steroid preferably is administered in a dose of from 25 to 500 mg, more preferably from 50 to 250 mg or from 100 to 150 mg, in particular in a dose of about 125 mg.

In a particular preferred embodiment of the invention the treatment of the patient with the anti-EGFR antibody is combined with a pre-medication with N-(4-hydroxyphenyl)acetamide and optionally methylprednisolone as follows in order to effectively reduce or prevent IRR:
(a) a first dose of 1000 mg of N-(4-hydroxyphenyl)acetamide the evening before the administration of the antibody,
(b) a second dose of 1000 mg of N-(4-hydroxyphenyl) 1 hour before the administration of the antibody and
(c) optionally one dose of 125 mg methylprednisolone 30 min before administration of the antibody.

In this scheme the reduced fucose anti-EGFR antibody is administered in doses described above; it is referred to the above disclosure.

In certain embodiments, no steroids are administered, preferably no steroids and no antihistamines are administered for reducing or preventing infusion related reactions caused by administration of the reduced fucose anti-EGFR antibody according to the invention. As described above, because of the advantageous glycosylation profile of the reduced fucose anti-EGFR antibody of the invention, allergic reactions, in particular IgE mediated, were not observed. No significant histamine or ECP release was observed. According to one embodiment, the infusion related reactions are treated or prevented only with the agent with analgesic and/or antipyretic properties.

In another aspect, the present invention provides an agent with analgesic and/or antipyretic properties for treating or preventing infusion related reactions caused by the administration of an anti-EGFR antibody. The anti-EGFR antibody preferably is the reduced fucose anti-EGFR antibody as defined herein. The features and embodiments of the other aspects of the invention accordingly apply to this aspect of the invention.

Another approach to reduce infusion-related reactions is the use of an improved administration scheme. As demonstrated by the experimental data provided herein, infusion-related reactions could effectively be prevented by splitting the first dose of the reduced fucose anti-EGFR antibody according to the present invention into two parts and administering the partial doses sequentially to the patient, preferably within a short time frame.

In certain preferred embodiments, the first dose of the reduced fucose anti-EGFR antibody according to the present invention is administered to the patient as two or more separate partial doses, in particular as two separate partial doses. Preferably all partial doses of the first dose are administered to the patient within 3 days, preferably within 2 days, in particular within 36 hours. In preferred embodiments, the first partial dose administered to the patient comprises 150 mg or less, preferably 100 mg or less, more preferably 80 mg or less, in particular about 60 mg of the reduced fucose anti-EGFR antibody. The first partial dose is preferably administered to the patient via infusion, in particular intravenous infusion, over a time period of at least 1 hour, preferably at least 1.5 hours, more preferably at least 2 hours, in particular about 2.5 hours. In preferred embodiments, the first dose is split into two separate partial doses. Preferably, the second partial dose comprises the remaining amount of the first dose of the reduced fucose anti-EGFR antibody which is not comprised in the first partial dose. The second partial dose is preferably administered to the patient via infusion, in particular intravenous infusion, within a time period of 10 hours or less, preferably 8 hours or less, more preferably 6 hours or less, in particular within about 5.5 hours. The first and the second partial doses of the first dose are preferably administered on two consecutive days.

In certain embodiments, in particular where the above administration scheme is used, no steroids are administered, preferably no steroids and no antihistamines are administered, more preferably also no agent with analgesic and/or antipyretic properties are administered for reduction or prevention of infusion related reactions. In particular, the infusion related reactions are reduced or prevented only by using the improved administration scheme. This is in particular advantageous since in specific cases it could be demonstrated that a premedication directed against IRRs, in particular a premedication with steroids and/or antihistamines and/or agents with analgesic and/or antipyretic properties may reduce the therapeutic acitivity of the reduced fucose anti-EGFR antibody according to the invention. Therefore, in certain advantageous embodiments the patient does not receive a premedication for reducing or preventing infusion related reactions caused by administration of the reduced fucose anti-EGFR antibody according to the invention. In another embodiment, the improved administration scheme is combined with the administration of an analgesic and/or antipyretic agent as described above.

Using the above-described premedication and in particular the improved administration scheme, infusion related reactions (IRRs) could be reduced. For example, in the clinical studies with the reduced fucose anti-EGFR antibody according to the invention, only about 50% of the patients showed IRRs which were mainly restricted to the first infusion of the antibody and did not reappear. The observed IRRs were also only of grade 1 or 2. Furthermore, the IRRs seen in the clinical studies so far did not include any allergic reaction.

### SPECIFIC EMBODIMENTS OF THE PRESENT INVENTION

Specific and particularly preferred embodiments of the present invention will be again described in the following:

In specific embodiments, the reduced fucose anti-EGFR antibody according to the present invention has the following characteristics:
(i) it comprises a heavy chain variable region comprising the amino acid sequence of SEQ ID NO: 7 or 9 or an amino acid sequence which is at least 80% identical thereto and comprising a CDR1 having the amino acid sequence of SEQ ID NO: 1, a CDR2 having the amino acid sequence of SEQ ID NO: 2, and a CDR3 having the amino acid sequence of SEQ ID NO: 3;
(ii) it comprises a light chain variable region comprising the amino acid sequence of SEQ ID NO: 8 or 10 or an amino acid sequence which is at least 80% identical thereto and comprising a CDR1 having the amino acid sequence of SEQ ID NO: 4, a CDR2 having the amino acid sequence of SEQ ID NO: 5, and a CDR3 having the amino acid sequence of SEQ ID NO: 6;
(iii) it has the following glycosylation characteristics in the CH2 domain:
   (a) a relative amount of glycans carrying a fucose residue of 30% or less, preferably 25% or less;
   (b) a relative amount of glycans carrying a bisecting GlcNAc of at least 5%, preferably at least 10%;
   (c) a relative amount of glycans carrying at least one galactose of at least 50%, preferably at least 60%; and
   (d) a relative amount of glycans carrying two galactoses of at least 10%, preferably at least 15%;
   (e) a relative amount of glycans carrying at least one sialic acid of at least 1%, preferably at least 2%; and
   (f) optionally a relative amount of glycans carrying two sialic acids of at least 0.5%, preferably at least 1%;
(iv) it optionally comprises a glycosylation site in the VH domain, wherein if said glycosylation site is present, it has the following glycosylation characteristics:
   (a) a relative amount of glycans carrying a fucose residue of 40% or less, preferably 35% or less;
   (b) a relative amount of glycans carrying a bisecting GlcNAc of at least 35%, preferably at least 40%;
   (c) a relative amount of glycans carrying at least one galactose of at least 85%, preferably at least 90%; and
   (d) a relative amount of glycans carrying at least two galactoses of at least 70%, preferably at least 80%;
   (e) a relative amount of glycans carrying at least one sialic acid of at least 50%, preferably at least 60%;
   (f) a relative amount of glycans carrying at least two sialic acids of at least 35%, preferably at least 45%.

In preferred embodiments, the reduced fucose anti-EGFR antibody according to the invention is administered in one week intervals with an initial dose in the range of from 900 mg to 1100 mg and with subsequent doses in the range of from from 600 mg to 850 mg. In another embodiment, the reduced fucose anti-EGFR antibody according to the invention is administered in intervals of two weeks with each dose being in the range of from 1250 mg to 1500 mg.

In specifically preferred embodiments, the reduced fucose anti-EGFR antibody according to the invention causes adverse skin reactions of grade 3 or higher in not more than 10% of the treated patients and/or causes acneiform skin rash in not more than 35% of the treated patients when administered in an amount of at least 240 mg per week for at least 6 weeks.

In certain embodiments, the reduced fucose anti-EGFR antibody according to the invention any is for treating a human patient
(i) who has been previously treated with cetuximab (Erbitux®), panitumumab (Vectibix®) and/or GA201, which caused an adverse skin reaction of grade 3 or higher in the patient;
(ii) with known severe adverse skin reaction of grade 3 or higher against cetuximab (Erbitux®), panitumumab (Vectibix®) and/or GA201;
(iii) with increased risk of developing a severe adverse reaction of grade 3 or higher during treatment with an cetuximab (Erbitux®), panitumumab (Vectibix®) and/or GA201;
(iv) which has been previously treated with an anti-EGFR antibody, and wherein said previous treatment was interrupted, terminated or wherein the dosage of the anti-EGFR antibody had to be reduced because an adverse skin reaction against said anti-EGFR antibody occurred during said treatment, wherein the anti-EGFR antibody is cetuximab (Erbitux®), panitumumab (Vectibix®) or GA201;
(v) which has been previously treated with an anti-EGFR antibody, and wherein said previous treatment with the anti-EGFR antibody is not or cannot be continued because an adverse skin reaction against said anti-EGFR antibody occurred, wherein the anti-EGFR antibody is cetuximab (Erbitux®), panitumumab (Vectibix®) or GA201.

In certain embodiments, the reduced fucose anti-EGFR antibody according to the invention is for treating an EGFR positive renal cell carcinoma in a human patient, wherein the reduced fucose anti-EGFR antibody is capable of inducing an antibody-dependent cellular cytotoxicity reaction in the patient; and wherein the reduced fucose anti-EGFR antibody causes adverse skin reactions of grade 3 or higher in not more than 10% of the treated patients and/or causes acneiform skin rash in not more than 35% of the treated patients. In further embodiments, the reduced fucose anti-EGFR antibody according to the invention is for treating a malignant pleural or peritoneal effusion in a human patient having an EGFR positive neoplastic disease, wherein the reduced fucose anti-EGFR antibody is capable of inducing an antibody-dependent cellular cytotoxicity reaction in the patient; and wherein the reduced fucose anti-EGFR antibody causes adverse skin reactions of grade 3 or higher in not more than 10% of the treated patients and/or causes acneiform skin rash in not more than 35% of the treated patients. In certain embodiments, the patient was treated previously with an anti-EGFR antibody causing such adverse skin reactions, such as e.g. Erbitux®, prior to the treatment with the reduced fucose anti-EGFR antibody and the preceding treatment was terminated or interrupted because of adverse skin reactions against said anti-EGFR antibody.

According to one embodiment, an anti-EGFR antibody with a glycosylation site in the CH2 domain is provided, wherein 50% or less of the glycans attached to said glycosylation site carry fucose (reduced fucose anti-EGFR antibody) and wherein the reduced fucose anti-EGFR antibody is capable of inducing an antibody-dependent cellular cytotoxicity reaction, for treating an EGFR positive neoplastic disease in a human patient, wherein the reduced fucose anti-EGFR antibody causes adverse skin reactions of grade 3 or higher in not more than 10% of the treated patients. According to one embodiment, the reduced fucose anti-EGFR antibody causes acneiform skin rash of grade 3 or higher in not more than 5% of the treated patients.

According to one embodiment, said reduced fucose anti-EGFR antibody is for treating a human patient that is afflicted with an EGFR positive neoplastic disease for which it has been shown that at least one other anti-EGFR antibody shows adverse skin reactions in more than 50% of the treated patients and/or adverse skin reactions of grade 3 or higher in at least 12% of the patients. According to one embodiment, said reduced fucose anti-EGFR antibody is for treating an EGFR positive neoplastic disease in a human patient, wherein treatment conditions are used which for at least one other anti-EGFR antibody cause an adverse skin reaction in at least 50% of the patients when using said other anti-EGFR antibody, or cause an adverse skin reaction of grade 3 or higher in at least 12% of the patients when using said other anti-EGFR antibody. According to one embodiment, said reduced fucose anti-EGFR antibody is for treating a human patient who has been previously treated with at least one EGFR inhibitor which caused an adverse skin reaction of grade 3 or higher in said patient. The EGFR inhibitor may be an anti-EGFR antibody such as cetuximab (Erbitux®), panitumumab (Vectibix®) and GA201.

The EGFR positive neoplastic disease may be an EGFR positive cancer selected from the group consisting of colon carcinomas, rectal carcinomas, non-small cell lung carcinomas, squamous cell lung cancer, renal cell carcinomas, triple negative breast cancer, squamous cell carcinomas of the head and neck, esophageal adenocarcinomas, gastric adenocarcinomas, gastroesophageal junction adenocarcinomas, endometrical carcinomas or sarcomas, cervical carcinomas.

According to one embodiment, said reduced fucose anti-EGFR antibody is for treating kidney cancer in a human patient, wherein, preferably, the kidney cancer is selected from clear cell renal cell carcinoma and papillary renal cell carcinoma.

According to one embodiment, said reduced fucose anti-EGFR antibody is for treating a malignant effusion in a human patient having an EGFR positive neoplastic disease. The effusion may be a pleural effusion or a peritoneal effusion and wherein the EGFR positive neoplastic disease is a gastric carcinoma or an adenocarcinoma of the esophageal/gastric junction.

According to a preferred embodiment, the reduced fucose anti-EGFR antibody comprises all of the following glycosylation characteristics in the glycosylation site of the CH2 domain:
(i) a relative amount of glycans carrying a fucose residue of 20% to 0%, preferably 15% to 3%;
(ii) a relative amount of glycans carrying a bisecting GlcNAc of at least 5%, preferably at least 10%;
(iii) a relative amount of glycans carrying at least one galactose of at least 50%, preferably at least 60%;
(iv) a relative amount of glycans carrying two galactoses of at least 10%, preferably at least 15%;
(v) a relative amount of glycans carrying at least one sialic acid, in particular NeuAc, of at least 1%, preferably at least 2%;
(vi) a relative amount of glycans carrying two sialic acids, in particular NeuAc, of at least 0.5%, preferably at least 1%; and
(vii) it does not comprise Galα1,3-Gal.

Furthermore, the reduced fucose anti-EGFR antibody may comprise an additional glycosylation site in the heavy chain variable region VH and may comprise all of the following glycosylation characteristics in the glycosylation site of the VH domain:
(i) a relative amount of glycans carrying a fucose residue of 40% or less, preferably 35% or less;
(ii) a relative amount of glycans carrying a bisecting GlcNAc of at least 35%, preferably at least 40%;
(iii) a relative amount of glycans carrying at least one galactose of at least 85%, preferably at least 90%;
(iv) a relative amount of glycans carrying at least two galactoses of at least 70%, preferably at least 80%;
(v) a relative amount of glycans carrying at least one sialic acid of at least 50%, preferably at least 60%;
(vi) a relative amount of glycans carrying at least two sialic acids of at least 35%, preferably at least 45%.

The reduced fucose anti-EGFR antibody described above may comprise a heavy chain variable region comprising a CDR1 having the amino acid sequence of SEQ ID NO: 1, a CDR2 having the amino acid sequence of SEQ ID NO: 2, and a CDR3 having the amino acid sequence of SEQ ID NO: 3; and may comprise a light chain variable region comprising a CDR1 having the amino acid sequence of SEQ ID NO: 4, a CDR2 having the amino acid sequence of SEQ ID NO: 5, and a CDR3 having the amino acid sequence of SEQ ID NO: 6.

According to one embodiment, the EGFR positive neoplastic disease comprises a K-RAS mutation.

According to one embodiment, the treatment with the reduced fucose anti-EGFR antibody includes the administration of the reduced fucose anti-EGFR antibody in an amount of from 250 to 1500 mg, preferably 350 to 1250 mg, per dose every week or less frequently.

According to certain embodiments, treatment of the EGFR positive neoplastic disease with the reduced fucose anti-EGFR antibody involves
(i) binding and activation of granulocytes, in particular neutrophil and eosinophil granulocytes, by the reduced fucose anti-EGFR antibody bound to cells of the EGFR positive neoplastic disease; and inducing said activated granulocytes to destroy said cells of the EGFR positive neoplastic disease; and/or
(ii) activation of macrophages by the reduced fucose anti-EGFR antibody bound to cells of the EGFR positive neoplastic disease; and inducing said activated macrophages to destroy said cells of the EGFR positive neoplastic disease.

According to certain embodiments, the reduced fucose anti-EGFR antibody is for treating a human patient
(i) with known severe adverse skin reaction of grade 3 or higher against an EGFR inhibitor which causes such severe adverse skin reactions;
(ii) with increased risk of developing a severe adverse reaction of grade 3 or higher during treatment with an EGFR inhibitor which causes severe adverse skin reactions of grade 3 or higher, in particular in more than 10% of the patients;
(iii) which has been previously treated with an EGFR inhibitor, and wherein said previous treatment was interrupted, terminated or wherein the dosage of the EGFR inhibitor had to be reduced because an adverse skin reaction against said EGFR inhibitor occurred during said treatment; and/or
(iv) which has been previously treated with an EGFR inhibitor, and wherein said previous treatment with the EGFR inhibitor is not or cannot be continued because an adverse skin reaction against said EGFR inhibitor occurred.

According to certain embodiments, the patient is homozygous for phenylalanine in amino acid position 158 of the Fcγ receptor IIIa (FcγRIIIa-158F/F), the patient is heterozygous for valine and phenylalanine in amino acid position 158 of the Fcγ receptor IIIa (FcγRIIIa-158V/F) or wherein the patient is homozygous for valine in amino acid position 158 of the Fcγ receptor IIIa (FcγRIIIa-158V/V) and wherein, preferably, the reduced fucose anti-EGFR antibody is for treatment of patients irrespective of their FcγRIIIa allotype.

According to certain embodiments, the reduced fucose anti-EGFR antibody is for treating a human patient which has developed an allergic reaction against an anti-EGFR antibody in a previous treatment and/or for treating a human patient with pre-existing Gal-Gal IgE antibodies.

According to certain embodiments, the reduced fucose anti-EGFR antibody is for treating a human patient which has been previously treated with one or more anti-cancer therapies and wherein the EGFR positive neoplastic disease is resistant to or has progressed after said previous treatment(s).

The invention in particular pertains to the following embodiments:

### Embodiment 1

An anti-EGFR antibody with a glycosylation site in the CH2 domain, wherein 50% or less of the glycans attached to said glycosylation site carry fucose (reduced fucose anti-EGFR antibody) and wherein the reduced fucose anti-EGFR antibody is capable of inducing an antibody-dependent cellular cytotoxicity reaction, for treating an EGFR positive neoplastic disease in a human patient, wherein the reduced fucose anti-EGFR antibody causes adverse skin reactions of grade 3 or higher in not more than 10% of the treated patients.

### Embodiment 2

The anti-EGFR antibody according to Embodiment 1, wherein the reduced fucose anti-EGFR antibody causes acneiform skin rash of grade 3 or higher in not more than 5% of the treated patients.

### Embodiment 3

The anti-EGFR antibody according to Embodiment 1 or 2, for treating a human patient that is afflicted with an EGFR positive neoplastic disease for which it has been shown that at least one other anti-EGFR antibody shows adverse skin reactions in more than 50% of the treated patients and/or adverse skin reactions of grade 3 or higher in at least 12% of the patients.

### Embodiment 4

The anti-EGFR antibody according to one or more of Embodiments 1 to 3, for treating an EGFR positive neoplastic disease in a human patient, wherein treatment conditions are used which for at least one other anti-EGFR antibody cause an adverse skin reaction in at least 50% of the patients when using said other anti-EGFR antibody, or cause an adverse skin reaction of grade 3 or higher in at least 12% of the patients when using said other anti-EGFR antibody.

### Embodiment 5

The anti-EGFR antibody according to one or more of Embodiments 1 to 4, for treating a human patient who has been previously treated with at least one EGFR inhibitor which caused an adverse skin reaction of grade 3 or higher in said patient.

### Embodiment 6

The anti-EGFR antibody according to Embodiment 5, wherein the EGFR inhibitor is an anti-EGFR antibody such as cetuximab (Erbitux®), panitumumab (Vectibix®) and GA201.

### Embodiment 7

The anti-EGFR antibody according to one or more of Embodiments 1 to 6, wherein the EGFR positive neoplastic disease is an EGFR positive cancer selected from the group consisting of colon carcinomas, rectal carcinomas, non-small cell lung carcinomas, squamous cell lung cancer, renal cell carcinomas, triple negative breast cancer, squamous cell carcinomas of the head and neck, esophageal adenocarcinomas, gastric adenocarcinomas, gastroesophageal junction adenocarcinomas, endometrical carcinomas or sarcomas, cervical carcinomas.

### Embodiment 8

The anti-EGFR antibody according to one or more of Embodiments 1 to 7, for treating kidney cancer in a human patient, wherein, preferably, the kidney cancer is selected from clear cell renal cell carcinoma and papillary renal cell carcinoma.

### Embodiment 9

The anti-EGFR antibody according to one or more of Embodiments 1 to 8, for treating a malignant effusion in a human patient having an EGFR positive neoplastic disease.

### Embodiment 10

The anti-EGFR antibody according to Embodiment 9, wherein the effusion is a pleural effusion or a peritoneal effusion and wherein the EGFR positive neoplastic disease is a gastric carcinoma or an adenocarcinoma of the esophageal/gastric junction.

### Embodiment 11

The anti-EGFR antibody according to one or more of Embodiments 1 to 10, wherein the reduced fucose anti-EGFR antibody comprises all of the following glycosylation characteristics in the glycosylation site of the CH2 domain:
(i) a relative amount of glycans carrying a fucose residue of 20% to 0%, preferably 15% to 3%;
(ii) a relative amount of glycans carrying a bisecting GlcNAc of at least 5%, preferably at least 10%;
(iii) a relative amount of glycans carrying at least one galactose of at least 50%, preferably at least 60%;
(iv) a relative amount of glycans carrying two galactoses of at least 10%, preferably at least 15%;
(v) a relative amount of glycans carrying at least one sialic acid, in particular NeuAc, of at least 1%, preferably at least 2%;
(vi) a relative amount of glycans carrying two sialic acids, in particular NeuAc, of at least 0.5%, preferably at least 1%;
(vii) it does not comprise Galα1,3-Gal.

### Embodiment 12

The anti-EGFR antibody according to one or more of Embodiments 1 to 11, in particular Embodiment 11, wherein the reduced fucose anti-EGFR antibody comprises an additional glycosylation site in the heavy chain variable region VH and comprises all of the following glycosylation characteristics in the glycosylation site of the VH domain:
(i) a relative amount of glycans carrying a fucose residue of 40% or less, preferably 35% or less;
(ii) a relative amount of glycans carrying a bisecting GlcNAc of at least 35%, preferably at least 40%;
(iii) a relative amount of glycans carrying at least one galactose of at least 85%, preferably at least 90%;
(iv) a relative amount of glycans carrying at least two galactoses of at least 70%, preferably at least 80%;
(v) a relative amount of glycans carrying at least one sialic acid of at least 50%, preferably at least 60%;
(vi) a relative amount of glycans carrying at least two sialic acids of at least 35%, preferably at least 45%.

### Embodiment 13

The anti-EGFR antibody according to one or more of Embodiments 1 to 12, comprising a heavy chain variable region comprising a CDR1 having the amino acid sequence of SEQ ID NO: 1, a CDR2 having the amino acid sequence of SEQ ID NO: 2, and a CDR3 having the amino acid sequence of SEQ ID NO: 3; and comprising a light chain variable region comprising a CDR1 having the amino acid sequence of SEQ ID NO: 4, a CDR2 having the amino acid sequence of SEQ ID NO: 5, and a CDR3 having the amino acid sequence of SEQ ID NO: 6.

### Embodiment 14

The anti-EGFR antibody according to one or more of Embodiments 1 to 13, wherein the EGFR positive neoplastic disease comprises a K-RAS mutation.

### Embodiment 15

The anti-EGFR antibody according to one or more of Embodiments 1 to 14, wherein the treatment includes the administration of the reduced fucose anti-EGFR antibody in an amount of from 250 to 1500 mg, preferably 350 to 1250 mg, per dose every week or less frequently.

### Embodiment 16

The anti-EGFR antibody according to one or more of Embodiments 1 to 15, wherein the treatment of the EGFR positive neoplastic disease with the reduced fucose anti-EGFR antibody involves
(i) binding and activation of granulocytes, in particular neutrophil and eosinophil granulocytes, by the reduced fucose anti-EGFR antibody bound to cells of the EGFR positive neoplastic disease; and inducing said activated granulocytes to destroy said cells of the EGFR positive neoplastic disease; and/or
(ii) activation of macrophages by the reduced fucose anti-EGFR antibody bound to cells of the EGFR positive neoplastic disease; and inducing said activated macrophages to destroy said cells of the EGFR positive neoplastic disease.

### Embodiment 17

The anti-EGFR antibody according to one or more of Embodiments 1 to 16, for treating a human patient
(i) with known severe adverse skin reaction of grade 3 or higher against an EGFR inhibitor which causes such severe adverse skin reactions;
(ii) with increased risk of developing a severe adverse reaction of grade 3 or higher during treatment with an EGFR inhibitor which causes severe adverse skin reactions of grade 3 or higher, in particular in more than 10% of the patients;
(iii) which has been previously treated with an EGFR inhibitor, and wherein said previous treatment was interrupted, terminated or wherein the dosage of the EGFR inhibitor had to be reduced because an adverse skin reaction against said EGFR inhibitor occurred during said treatment; and/or
(iv) which has been previously treated with an EGFR inhibitor, and wherein said previous treatment with the EGFR inhibitor is not or cannot be continued because an adverse skin reaction against said EGFR inhibitor occurred.

### Embodiment 18

The anti-EGFR antibody according to one or more of Embodiments 1 to 17, having one or more of the following characteristics:
(i) the patient is homozygous for phenylalanine in amino acid position 158 of the Fcγ receptor IIIa (FcγRIIIa-158F/F), the patient is heterozygous for valine and phenylalanine in amino acid position 158 of the Fcγ receptor IIIa (FcγRIIIa-158V/F) or wherein the patient is homozygous for valine in amino acid position 158 of the Fcγ receptor IIIa (FcγRIIIa-158V/V) and wherein, preferably, the reduced fucose anti-EGFR antibody is for treatment of patients irrespective of their FcγRIIIa allotype;
(ii) the reduced fucose anti-EGFR antibody is for treating a human patient which has developed an allergic reaction against an anti-EGFR antibody in a previous treatment and/or for treating a human patient with pre-existing Gal-Gal IgE antibodies; and/or
(iii) the reduced fucose anti-EGFR antibody is for treating a human patient which has been previously treated with one or more anti-cancer therapies and wherein the EGFR positive neoplastic disease is resistant to or has progressed after said previous treatment(s).

Numeric ranges described herein are inclusive of the numbers defining the range. The headings provided herein are not limitations of the various aspects or embodiments of this invention which can be read by reference to the specification as a whole. According to one embodiment, subject matter described herein as comprising certain steps in the case of methods or as comprising certain ingredients in the case of compositions refers to subject matter consisting of the respective steps or ingredients. It is preferred to select and combine preferred aspects and embodiments described herein and the specific subject-matter arising from a respective combination of preferred embodiments also belongs to the present disclosure.

This application claims the priorities of EP 13 002 106.6 and EP 13 002 108.2, both filed on Apr. 22, 2013, which are enclosed herein by reference in their entirety.

### FIGURES

FIG. 1 shows the IP-10 response in exemplary patients within 24 h after 1^{st} infusion of the reduced fucose anti-EGFR antibody. Serum concentrations above 1 ng/ml are already elevated levels and values >10 ng/ml are considered as strong responses.

FIG. 2 shows the cytokine release in a whole blood sample of exemplary patients after incubation with the indicated antibody or PBS for 4 h.

FIG. 3 shows the binding of Fuc− cetuximab (invention), Fuc+ cetuximab (Erbitux®) and a control human IgG1 antibody (hIgG1) to different human blood cell types. (A) Binding to different blood cell types sorted according to their expression of cluster of differentiation (CD) genes. (B) Detailed presentation of the lower axis area of A.

FIG. 4 shows the production of reactive oxygen species in a whole blood sample after incubation with different concentrations of Fuc− cetuximab (invention) or Fuc+ cetuximab (Erbitux®) in the presence (+ A-431) or absence (− A-431; control) of target cells expressing EGFR. (A) Percentage of cells showing staining for reactive oxygen species. (B) Concentration of reactive oxygen species in the blood sample.

FIG. 5 shows the secretion of lactoferrin in a whole blood sample after incubation with different concentrations of Fuc− cetuximab (invention) or Fuc+ cetuximab (Erbitux®) in the presence (+ A-431) or absence (− A-431; control) of target cells expressing EGFR. Indicated is the concentration of lactoferrin in the plasma.

FIG. 6 shows a schematic drawing of the biantennary complex-type structure of the carbohydrate chains which are attached to the glycosylation sites in the CH2 and optionally VH domains of the antibody. A black square represents an N-acetylglucosamine residue (GlcNAc), a gray circle represents a mannose residue (Man), a white circle represents a galactose residue (Gal), a gray rhombus represents a sialic acid residue (SA), a black triangle represents a fucose residue (Fuc) and a gray square represents a bisecting N-acetylglucosamine residue (bisGlcNAc). In the biantennary complex-type structure, Gal and SA in the branches of the carbohydrate, bisGlcNAc as well as Fuc are only optionally present in the carbohydrate structure and may also be absent.

### EXAMPLES

### Example 1

### Glycosylation Analysis of Cetuximab Variants

A reduced fucose anti-EGFR antibody according to the present invention, here a low fucosylation variant of cetuximab (Fuc− cetuximab) was obtained by expression in a human myeloid leukemia cell line which was derived from the cell line GT-5s (DSM ACC 3078) but wherein said cell line was altered to have a reduced fucosylation activity by modifying GMD expression. The high fucose anti-EGFR antibody cetuximab (Fuc+ cetuximab) was produced in the mouse myeloma cell line SP2/0 and thus, substantially corresponds to cetuximab (Erbitux®). This antibody was also used as reference in the subsequent in vitro studies (Examples 4, 6, 7 and 8).

To characterize the glycosylation pattern of the Fuc− cetuximab in more detail, glycoprofiling studies were performed. The chimeric human/mouse IgG1 antibody cetuximab comprises one N-glycosylation site in the heavy chain constant region 2 (Fc glycosylation site) and one N-glycosylation site in the heavy chain variable region (Fab glycosylation site). For glycoprofiling, the intact N-glycans were released from the protein core and the reducing ends of N-glycans were labeled with a fluorescence marker. The purified sample of the labeled N-glycans was separated by UPLC. Peak areas based on fluorometric detection were employed for calculation of the relative molar abundances of the N-glycan structures. Estimated data for the overall glycosylation on all N-glycoslylation sites of the antibody are summarized in Table 1. The values represent the relative molar contents of N-glycans containing the type of monosaccharide of interest (e.g. fucose).

TABLE 1

| | Rel. abundance [mol %]* | | | | | | | |
|---|---|---|---|---|---|---|---|---|
| Sample | F | S > 0 | S2 | G > 0 | G2 | B | M | Gal |
| Fuc⁻ cetuximab | 7 | 34 | 19 | 90 | 56 | 36 | 0 | 0 |
| Fuc⁺ cetuximab | 81 | 8 | 0 | 52 | 20 | 0 | 18 | 18 |

*Relative abundances of glycan structures are related to the total amount of N-glycans. F = fucosylated N-glycans; S > 0 = sialylated N-glycans; S2 = N-glycans with two sialic acids; G > 0 = galactosylated N-glycans; G2 = N-glycans with two galactoses; B = N-glycans with bisecting N-acetylglucosamine; M: high mannose-type and hybrid-type N-glycans; Gal: Galili epitope-bearing N-glycans.

The glycoprofiling shows that Fuc− cetuximab has a much lower fucose content and a higher bisGlcNAc, silaic acid and galactose content compared to the Fuc+ cetuximab expressed in mouse SP2/0 cells (as are used for the production of Erbitux®). Additionally, the Fuc+ cetuximab shows a significant amount of high mannose-type and hybrid-type N-glycans while the Fuc− cetuximab is exclusively glycosylated with complex-type N-glycans. Furthermore, the Fuc− cetuximab had due to the production in a human cell line a human glycosylation profile and thus, no detectable NeuGc and no detectable Galα1,3-Gal (Galili epitope).

To further characterize the Fuc− cetuximab, also the glycosylation patterns at the different glycosylation sites (Fab glycosylation and Fc glycosylation) were analyzed. For a separate determination of the glycosylation profiles at the Fc and Fab glycosylation sites, respectively, the antibody was digested and separated into a Fab and a Fc part prior to the glycan analysis.

TABLE 2

| | Rel. abundance [mol %]* | | | | | |
|---|---|---|---|---|---|---|
| Sample | F | S > 0 | S2 | G > 0 | G2 | B |
| Fuc⁻ cetuximab Fab part | 8 | 89 | 56 | 99 | 94 | 77 |
| Fuc⁻ cetuximab Fc part | 9 | 11 | 3 | 87 | 41 | 18 |

*Relative abundances of glycan structures are related to the total amount of N-glycans. F = fucosylated N-glycans; S > 0 = sialylated N-glycans; S2 = N-glycans with two sialic acids; G > 0 = galactosylated N-glycans; G2 = N-glycans with two galactoses; B = N-glycans with bisecting N-acetylglucosamine.

Target binding, specificity, affinity and Fv mediated anti-tumor activity of the Fuc− cetuximab and the Fuc+ cetuximab (Erbitux®) were analyzed in different comparability studies (see also examples below), in particular EGFR antigen ELISA, flow cytometry analysis, EGFR downmodulation, reduction of VEGF production, inhibition of tumor proliferation and the induction of tumor apoptosis. The results confirmed that the Fuc− cetuximab according to the present invention shows full maintenance of tumor cell proliferation inhibition and induction of tumor cell apoptosis. Therefore, the Fuc− cetuximab and the Fuc+ cetuximab are basically equivalent in binding and Fv mediated anti-tumor properties. Thus, the improvements regarding the therapeutic efficacy and in particular the anti-metastatic activity are related to the improved glycosylation characteristics of the reduced fucose anti EGFR antibody.

The Fuc− cetuximab as described in example 1 was used in the subsequent analyses and examples.

### Example 2

### Clinical Studies

A clinical phase I dose escalation study of Fuc− cetuximab (see example 1) as monotherapy in patients with locally advanced or metastatic EGFR-positive solid malignancies was performed. Patients with several different tumor types were included in the study, encompassing patients with colon cancer, lung cancer, gastric cancer, esophageal cancer, renal cell cancer including clear cell renal cell cancer and papillary renal cell cancer, gallbladder cancer, ovarian cancer and rectum cancer. A weekly or 2-weekly dosing scheme was used. The patients received either 12 mg, 60 mg, 120 mg, 240 mg, 480 mg, 720 mg, 990 mg or 1370 mg of the Fuc− cetuximab. All patients had a progressive disease state prior to the start of the clinical study. The treatment was safe and very well tolerated with mild to moderate infusion-related reactions (IRR) only at first infusion which can be controlled by paracetamol and/or an improved administration scheme.

An impressive therapeutic efficacy was seen in the late stage patients for the treatment of a variety of different tumors. In particular, a stabilization of the disease status could be obtained for up to 870 days in tumor types such as colon (including KRAS mutant as well as KRAS wildtype tumors), rectum, gastric, kidney/renal cell, lung, ovarian and penis cancer. At least stabilization of the disease status (clinical benefit) was achieved in more than 50% of all patients included in the study with an average duration of the stable disease of about 6 months. Of those patients who received at least 8 doses (or 4 biweekly) of the Fuc− cetuximab according to the invention, even 82% showed a clinical benefit. A partial or even complete response was observed for about 18% of the patients with Fuc− cetuximab as single agent therapy in the progressive patients with advanced disorder and heavy pretreatments. This is a remarkable effect as these patients have a particularly poor prognosis. Thus, they are not comparable to patients that receive treatment for the first or second time. 46% of all patients showed tumor reduction. Furthermore, a full reduction of effusions in lung or peritoneum was obtained. Major clinical benefit was seen in colon cancer, including patients after Erbitux® failures and progressors in K-Ras wild type as well as K-Ras mutant patients, and non-typical anti-EGFR antibody indications such as NSCLC, clear and non-clear renal cell carcinomas, gastric and esophageal cancer, and gallbladder. Therefore, the reduced fucose anti-EGFR antibody provides important new treatment options.

Concerning the different cancer types, the longest duration for a stable disease for colon cancer was about 400 days, with an average duration of about 180 days. About 80% of the colon cancer patients showed a clinical benefit, including patients progressive upon Erbitux® treatment and patients with a K-Ras mutant tumor. Patients with renal cell carcinoma showed an average duration of stable disease of about 240 days, the longest duration seen so far being up to 400 days.

Particularly noteworthy is the fact that all patients with renal cell carcinoma included in the study showed a stabilizing of the disease, which was in all cases obtained already with low amounts of 12 mg to 120 mg Fuc− cetuximab per week. A reduction of the sum of tumor lesions up to 35% was observed for renal cell carcinoma. The longest duration of a progression-free survival of the patients with renal cell carcinoma was so far up to 400 days.

Also patients with lung cancer or gastric/esophageal cancer showed remarkable responses to the Fuc− cetuximab antibody according to the invention. Both groups include patients with a complete response. A still ongoing treatment of a non-small cell lung cancer patient results in a stabile disease for now more than 870 days. Furthermore, the treatment with the Fuc− cetuximab antibody resulted in strong reduction of tumor markers and complete reduction of peritoneal effusions (ascites) in patients with gastric or esophageal cancer.

These clinical results are especially remarkable since it is the result of all dose cohorts including the patients receiving only 12 mg of the Fuc− cetuximab per week. In particular, a stable disease was observed in more than 70% of the patients which were treated at low Fuc− cetuximab doses (12 mg to 240 mg). Furthermore, more than 40% of all patients showed a tumor reduction in the target lesions. The treatment with the Fuc− cetuximab also reduced pleural and peritoneal effusions in cancer patients affected thereby.

Another astonishing result of the clinical study was the remarkably low rate of adverse reactions caused by the anti-EGFR antibody which allows the new therapeutic treatments described herein. Conventional anti-EGFR antibodies such as Erbitux® cause adverse skin reactions, in particular rash, in 80% to 90% of the treated patients. About 15% or more of the skin reactions are severe (at least grade 3), including cases with skin necrosis. Furthermore, nearly all patients treated with the Fuc+ cetuximab Erbitux® suffer under a progressive magnesium loss, leading to hypomagnesemia in up to 55% of the patients (see, e.g. the documents accompanying the marketing authorization for Erbitux® of the European Medicine Agency (EMEA) and the U.S. Food and Drug Administration (FDA), and the Cetuximab Cancer Drug Manual of the British Columbia Cancer Agency). In contrast to this, only about 40% of the patients of the present study developed skin reactions, most of which were only mild reactions (grade 1) and the remaining were moderate (grade 2). No grade 3 or 4 adverse skin reactions occurred. In particular, only 24% of the patients suffered from acneiform skin rashes and even those patients only developed mild forms. No acneiform skin rashes of grade 3 or higher were observed. Furthermore, decreased magnesium and potassium levels were observed during the clinical study with the Fuc− cetuximab in less than 8% and less than 9%, respectively, of the patients. No allergic reactions against the Fuc− cetuximab were observed which is attributable to the improved glycosylation. Also a lower incidence of other adverse side effects such as diarrhea was observed for the anti-EGFR antibody according to the invention when compared with Erbitux®. In particular, only 24% of the treated patients had adverse events of grade 3 or higher, none of which were adverse skin reactions. No interruption, delay in the administration, or reduction of the dosage was necessary during the clinical study because of such side effects. It is believed that the overall improved glycosylation pattern of the Fuc− antibody according to the present invention and also the differences in binding of the various human blood cell types which are apparently associated with the improved glycosylation pattern (see Example 6) could be responsible for this advantageous adverse reaction profile based on which novel treatment opportunities are provided as are described herein.

Selected records of patients who had a major response upon administration of the reduced fucose anti-EGFR antibodies according to the present invention, here Fuc− cetuximab (see example 1) are described in the following examples.

### Example 3

### Exemplary Cases of EGFR Positive Cancer Patients Treated with the Fuc− Cetuximab

### Exemplary Case 1

A patient (FcγRIIIa-158V/V) with advanced colon cancer (KRAS wt) and lung, liver and peritoneal metastases was treated with 1370 mg Fuc− cetuximab after the following preceding treatments
1. FOLFOX and bevacizumab
2. FOLFIRI and Fuc+ cetuximab (Erbitux®)
3. FOLFOX
4. dacarbazine
5. capecitabine and mytomicin

During the treatment with Fuc− cetuximab, the patient experienced a normalization of the liver function and a significant decrease in tumor markers (Ca19.9 decreased from 362 to 150 U/ml and CEA decreased from 398 to 15 ng/ml) which indicated treatment efficacy. The computed tomography (CT) scans documented a 26.2% decrease in tumor burden. The treatment was well tolerated, since the patient just developed a grade 1 acneiform rash and a grade 1 decrease in magnesium level.

### Exemplary Case 2

A patient (FcγRIIIa-158F/F) with advanced HER2 negative gastric cancer (miliary peritoneal carcinomatosis and ascites) was treated with 1370 mg Fuc− cetuximab after unsuccessful third-line chemotherapy with FOLFOX. After the first 2 months of the treatment with Fuc− cetuximab, the patient experienced a significant decrease in tumor marker (Ca19.9: 2359 to 443 U/ml) which indicated treatment efficacy. The measurable lesion at a mesenteric node was reduced from 13×11 cm to 12×8 cm. Furthermore, a significant reduction in abdominal effusion was achieved, which is almost not detectable any more. The treatment was optimally tolerated, since the patient just developed a grade 1 acneiform rash. The magnesium levels remained within the normal ranges.

### Exemplary Case 3

A patient (FcγRIIIa-158F/F) with adenocarcinoma of the esophageal/gastric junction including pleural and peritoneal metastases was treated with 1370 mg Fuc− cetuximab weekly. The cancer does not show HER2 overexpression, however, EGFR gene amplification was detected by FISH. The patient additionally suffered from pleural and peritoneal effusions which had to be punctured and drained frequently. Since institution of the therapy with the Fuc− cetuximab, the pleural effusion did not need draining again and the patient's general condition improved markedly. Already after one cycle of Fuc-cetuximab treatment, the tumor markers dropped significantly (tumor marker Ca19-9 dropped from 1769 to 204 U/ml and tumor marker CEA dropped from 444 to 104 ng/ml). A full reduction of all tested tumor markers to normal levels (Ca19-9: 12.4 U/ml and CEA: 4.4 ng/ml) in combination with full reduction of abdominal effusion to no more measurable ascites was reached, resulting in a patient with a complete clinical response.

### Exemplary Case 4

A patient (FcγRIIIa-158V/V) with an advanced non-small cell lung cancer was treated with 720 mg Fuc− cetuximab weekly and developed a complete response for >397 days confirmed by radiology and CT scans. The patient had previous bronchioscopy, as well as radio-chemotherapy (with cisplatin and vinorelbin) and chemotherapy (with docetaxel) treatments over 1 year when he started treatment with Fuc− cetuximab. The treatment was very well tolerated during the first 18 infusions with accompanying premedication as well as during the further infusions without premedication. The tumor lesions stayed constant during the first infusions. Then the tumor lesions started to shrink and after ˜10.5 months of treatment start reached a complete response according to RECIST 1.1. The disappearance of all tumor lesions was confirmed in 2 consecutive CT scans within 8 weeks. The general health condition is good and the patient is even working again. This is a highly remarkable event since normally the health condition and prognosis of patients having a comparable initial cancer status are far remote from the possibility of pursuing their profession. The therapy is presently ongoing.

### Exemplary Case 5

A patient (FcγRIIIa-158F/F) with papillary renal cell carcinoma was treated with 120 mg Fuc− cetuximab weekly. The summed up area of the target lesions was reduced by the Fuc− cetuximab treatment by 35%, with a 45% reduction in respect to the maximum size of the sum of the target lesions.

### Exemplary Case 6

A patient having an advanced colon cancer with lung, liver and lymph node metastasis (after two prior treatments with FOLFIRI plus Avastin (bevacizumab) and another chemotherapy) was treated with 990 mg every 2^{nd} week. The patient showed a partial response according to RECIST 1.1 in the 1^{st} scan with a ˜35% reduction of sum of tumor target lesions in combination with rapid symptomatic relief and normalization of lab tests (liver function) and CEA level. The treatment was optimally tolerated, since the patient only developed a mild skin toxicity.

### Exemplary Case 7

A patient (FcγRIIIa-158F/V) with papillary renal cell carcinoma was treated with 60 mg Fuc− cetuximab weekly. The patient showed a stable disease for more than one year.

### Exemplary Case 8

A patient (FcγRIIIa-158F/F) with clear cell renal cell carcinoma with mutant KRAS was treated with 120 mg Fuc− cetuximab weekly. The tumor showed merely slow growth after treatment for 4 months.

### Exemplary Case 9

A patient (FcγRIIIa-158F/F) with clear cell renal cell carcinoma was treated with 12 mg Fuc− cetuximab weekly. Tumor growth was stopped for 5 months, after which merely a slow growth was detected.

### Example 4

### Involvement of NK Cells and Macrophages in the Response to Fuc− Cetuximab

To analyze the involvement of natural killer cells (NK cells) and macrophages in the therapeutic activity of the Fuc− cetuximab according to the invention, serum samples were obtained from all patient of the clinical study before, during and after administration of the antibody.

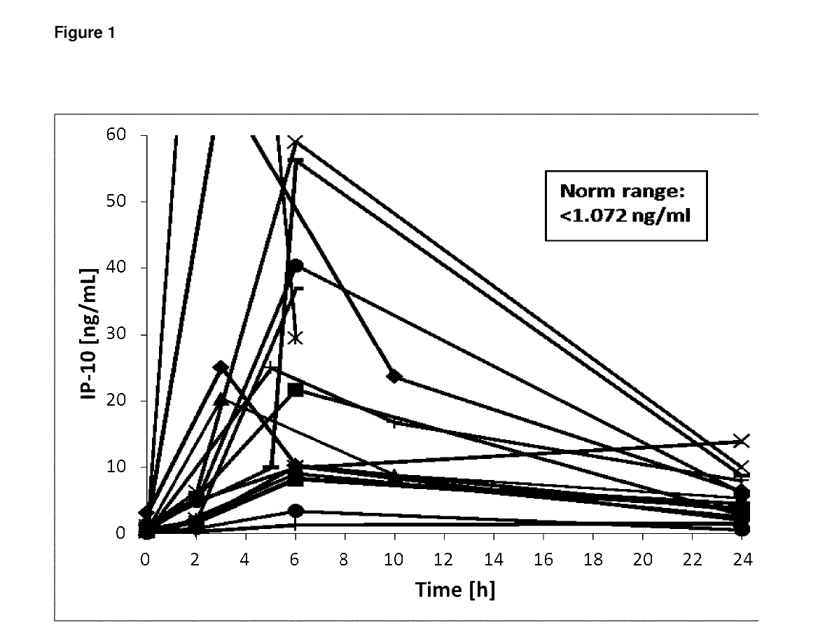

IFN-γ was analyzed as an indicator of the Fuc− cetuximab induced stimulation of NK cells, a major source of elevated IFN-γ levels. A low degree of IFN-γ induction was observed in slightly more than 50% of the patients. Since IFN-γ is known to be induced very transiently, the IFN-γ-induced protein 10 (IP-10) was analyzed additionally in several patients. IP-10 is secreted by macrophages upon stimulation with IFN-γ and serves as a more stable indicator of IFN-γ secretion by NK cells and activation of macrophages. A fast and strong stimulation of IP-10 secretion was observed in most patients (88%) within 6 h after start of infusion (FIG. 1).

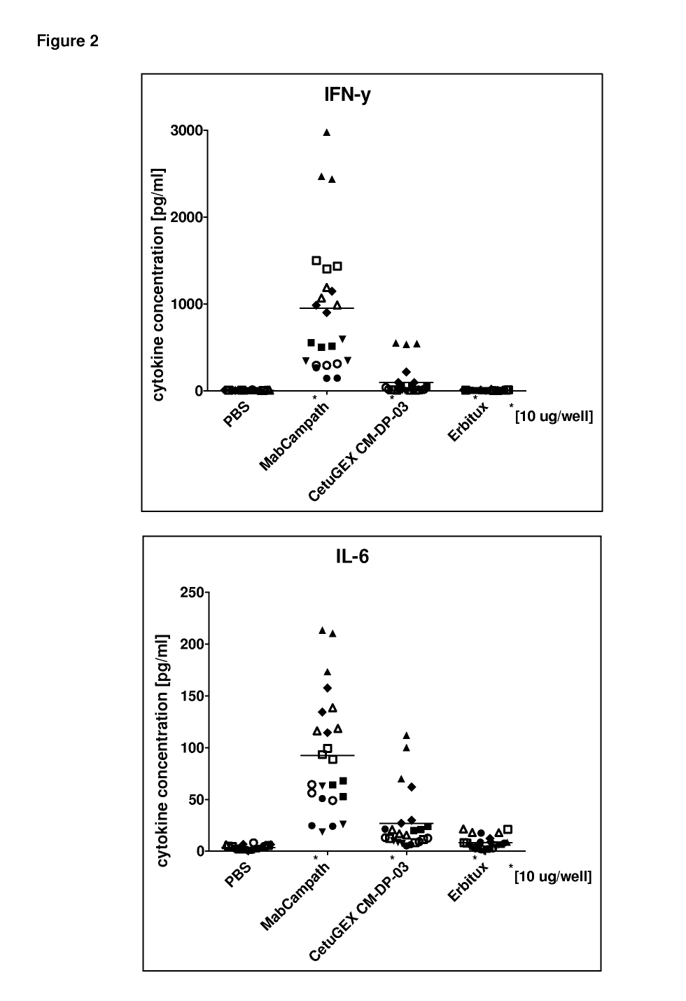

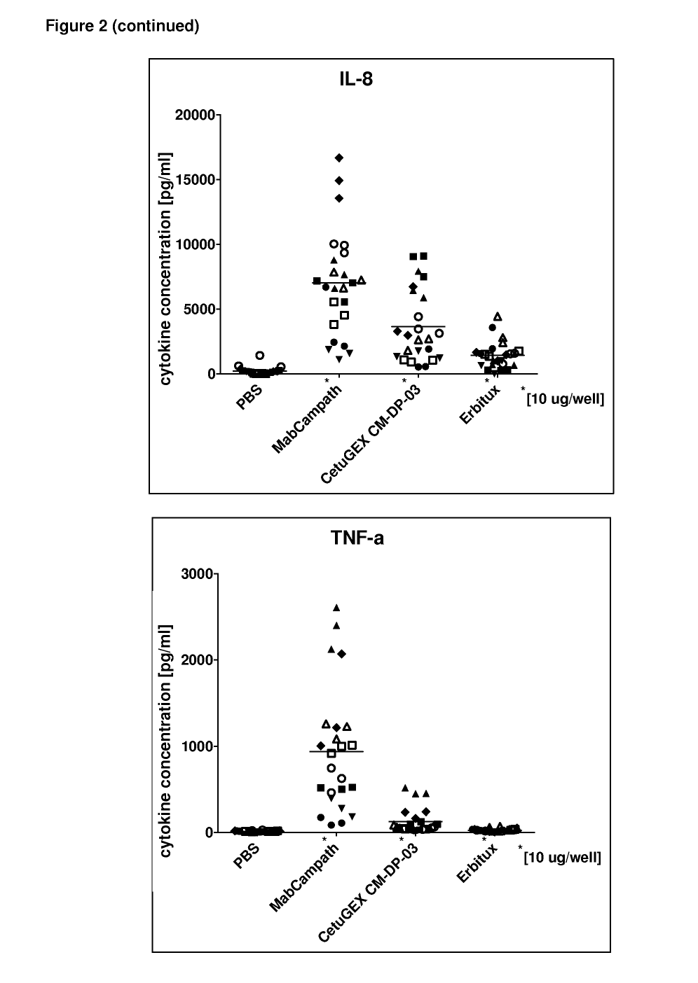

Furthermore, the induction of various cytokines by the Fuc− cetuximab antibody was also analyzed in vitro by incubating blood samples of the patients taken prior to the antibody treatments with the Fuc− cetuximab according to the invention, Fuc+ cetuximab, the antibody MabCampath directed against B- and T-lymphocyte epitopes as positive control and PBS as negative control. As result, an increase of the cytokines IFN-γ, IL-6, IL-8 and TNF-α could be observed (FIG. 2).

The induction of IFN-γ and IP-10 release indicates the involvement of macrophages and NK cells in the response to Fuc− cetuximab. Furthermore, the induction of the release of these and the further cytokines also is n indicator for the therapeutic efficacy of the Fuc− cetuximab antibody used in the clinical study. The induced cytokines demonstrate that an immune reaction is triggered by the antibody and furthermore activates the patient's immune system for the following antibody treatment.

### Example 5

### Prevention of Infusion Related Reactions

In the clinical study for assessing the therapeutic activity of Fuc− cetuximab, also mild to moderate adverse reactions caused by infusion of the Fuc− cetuximab (infusion related reactions, IRR) were observed during the first infusion in patients of the first cohorts. To prevent IRR, the remaining patients were pretreated with paracetamol prior to the Fuc− cetuximab infusion. Paracetamol pretreatment involved one dose of 1000 mg the evening before and one dose of 1000 mg 1 h before the Fuc− cetuximab infusion. The pretreatment of the patients resulted in a decrease in IRR.

In a second approach, the administration scheme of the first infusion was optimized to reduce IRRs. According to this optimized administration scheme, the first dose is split into two infusions given on two consecutive days. The first infusion contains 60 mg of the Fuc− cetuximab and is administered over 2.5 hours. The second infusion contains the remaining amount of the Fuc− cetuximab of the first dose and is administered on the next day by infusion within 5.5 hours. By improving the administration scheme of the first infusion, the occurrence of IRR could be further reduced to 50%. This dosing regimen also allowed a minimization of the premedication to the first infusion and if an IRR occurs to the one next infusion (only) following the IRR. This is important because the premedication, especially steroids, are described in the literature to inhibit NK-cell and ADCC activity which are the key functions of Fuc− cetuximab, as well as a reduction of macrophage and neurophile activity, and monocyte counts. In patients which were treated with less premedication the anti-EGFR antibody of the invention also showed a higher anti-tumor efficacy.

Furthermore, it is to be noted that during the entire clinical study, using premedication or the improved administration scheme infusion related reactions were predominantly observed during the first infusion and were only of mild or moderate severity (grade 1 or 2). In addition, the treated patients did not show an allergic reaction against the anti-EGFR antibody according to the invention.

These findings demonstrate that IRR caused by Fuc− cetuximab infusion can be prevented by pretreatment with an analgesic agent such as paracetamol and/or by using an optimized administration schedule for the first dose.

### Example 6

### Analysis of Differently Fucosylated Antibody Variants to the Antigen EGFR in Preclinical In Vitro and In Vivo Studies

The Fuc− cetuximab antibody according to the invention was characterized in several in vitro and in vivo studies prior to performing the clinical studies. In particular, also a comparison with the Fuc+ cetuximab antibody was done. These studies involved the binding of the antibodies to and their effect on the antigen EGFR, their biological effects (including ADCC) on different tumor cells expressing EGFR, and their efficacy in in vivo tumor models.

### Antigen Binding Assay

Antigen binding of the Fuc− cetuximab antibody was determined and compared to Fuc+ cetuximab in ELISA assays using immobilized EGFR and by cell cytometry by staining EGFR-expressing cells for binding of the antibody. In these in vitro assays, the Fuc-cetuximab antibody according to the invention showed strong binding to EGFR which was comparable to the binding of Fuc+ cetuximab.

### Direct Effects on EGFR-Expressing Cells

The activity of Fuc− cetuximab and Fuc+ cetuximab to inhibit EGFR phosphorylation and to induce apoptosis in human tumor cells expressing EGFR was determined. Upon activation by ligand binding, EGFR dimerizes and is autophosphorylated. The inhibition of EGFR activation due to binding of cetuximab can be analyzed by determining the reduction in phosphorylation of EGFR. This was measured by staining phosphorylated EGFR with a specific antibody. The analysis showed that Fuc− cetuximab according to the invention as well as Fuc+ cetuximab effectively inhibit EGFR phosphorylation in the EGFR-expressing tumor cell lines A431 (human epidermoid carcinoma cell line of the vulva) cells and LS174T (human epithelial colon adenocarcinoma). Furthermore, both antibodies are capable of inducing apoptosis in EGFR-expressing tumor cells. This was analyzed by measuring the release of mitochondrial dehydrogenases upon contacting of A431 cells with the antibody. The activity of Fuc− cetuximab according to the invention as well as Fuc+ cetuximab in inhibition of EGFR phosphorylation and induction of apoptosis were shown to be comparable.

### ADCC Assays

Reduction of fucose content within the glycosylation site in the antibody Fc domain is reported to lead to an increase of ADCC activity, the antibody-dependent cellular cytotoxicity resulting in a specific lysis of antigen positive tumor cells. This effect is caused by the higher affinity binding of the fucose-reduced antibody to the FcγRIIIa receptor on natural killer cells. Two allotypes of this receptor at amino acid position 158 (V158F) are known which have different affinities to human IgG1 with the V allele having a higher affinity to human IgG1 than the F allele receptor. Therefore, the ADCC activity of Fuc− cetuximab in comparison to Erbitux® on homozygeous VV, homozygeous FF and heterozygeous FV donors was analyzed, using a europium release assay with EGFR-positive tumor cell lines (LS174T, SKOV3, SK-BR3) as target cells.

The results show that Fuc− cetuximab mediates ADCC activities that are strongly enhanced compared to Fuc+ cetuximab on all donors. The maximal lysis achieved with Fuc− cetuximab was significantly increased compared to Fuc+ cetuximab and the concentration of Fuc− cetuximab necessary to achieve a specific lysis rate is strongly decreased. This is even more pronounced when considering the effector cells of donors having a VF or FF genotype of the FcγRIIIa receptor.

In a further ADCC assay, cells of the human lung adenocarcinoma epithelial cell line A549 were used as target cells. The K-Ras gene in these cells comprises a mutation in codon 12 leading to a constitutively active K-Ras protein having a Gly-12-Ser mutation. As result, it was demonstrated that the Fuc− cetuximab according to the invention is capable of inducing target cell lysis via ADCC even for target cancer cells which comprise a constitutively active EGFR signal transduction pathway, i.e. which cannot be treated by blocking EGFR ligand binding.

### In Vivo Pharmacological Studies

Several in vivo studies were performed in mice and cynomolgus monkeys to investigate the pharmacological effects of Fuc− cetuximab, some of them were performed in comparison to Fuc+ cetuximab. The antitumor activity of Fuc− cetuximab was studied in athymic nude mice xenografted with EGFR positive tumor cells from a human cell line (A431, DU145) or from patient derived carcinoma xenografts of NSCLC (non-small cell lung cancer) and CRC (colorectal cancer) origin.

It could be shown that both antibodies, the Fuc− cetuximab as well as the Fuc+ cetuximab, inhibit tumor growth dose-dependently compared to PBS treated animals. No significant difference between the relative tumor volume in the Fuc− cetuximab treated group and the Fuc+ cetuximab treated group was found in any of the dose groups. Comparable efficacies of Fuc− cetuximab and Fuc+ cetuximab were expected since the advantage of the increased ADCC activity of Fuc− cetuximab is not relevant in mice (mice do not have the FcγRIIIa allotype variations like humans).

Treatment of mice administered with the highly EGFR expressing tumor NSCLC #7466 and CRC #8060 with Fuc− cetuximab resulted in a strong tumor growth inhibition and even a significant percentage of tumor remissions. Furthermore, it was demonstrated that Fuc− cetuximab according to the invention effectively inhibits growth of the tumor cells CRC #8397 (patient-derived colorectal carcinoma cells) which carry the oncogenic, constantly active G12D mutant of K-Ras.

As demonstrated, Fuc− cetuximab according to the invention inhibits tumor growth independent of the K-Ras mutational status of the tumor cells.

### Example 7

### Binding of the Differently Fucosylated Antibody Variants to Human Blood Cells

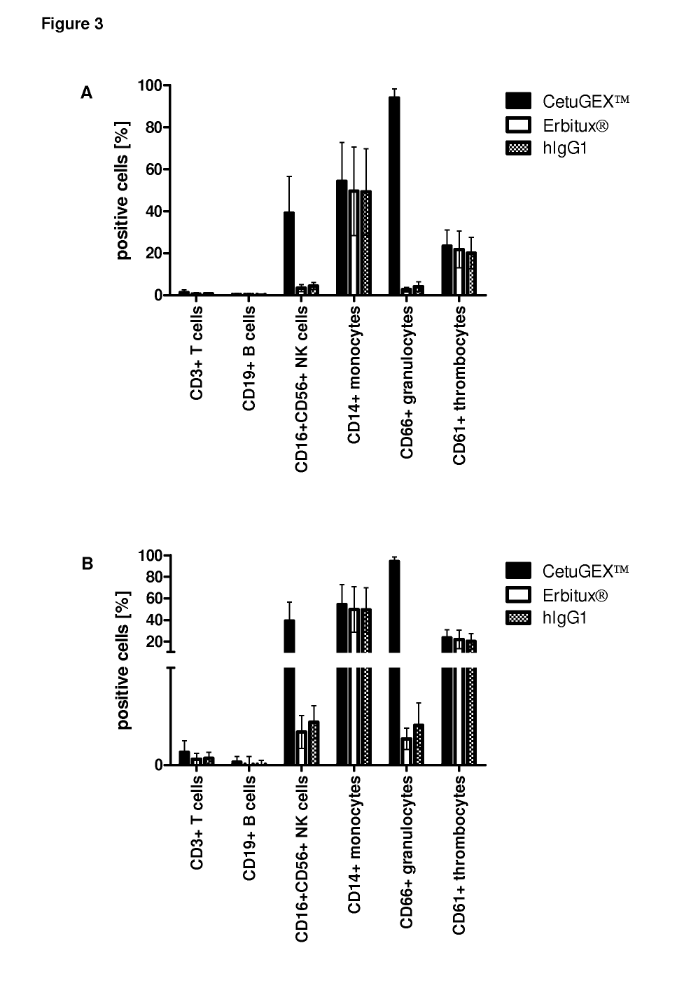

To evaluate differences in the behaviour of the differently fucosylated anti-EGFR antibody variants in the human body, the binding of said antibodies to various human blood cells was analyzed. In this respect, human blood cells were stained with Fuc− cetuximab, Fuc+ cetuximab or irrelevant human IgG1 antibody and sorted for different blood cell types. Briefly, binding of Fuc− cetuximab and Fuc+ cetuximab to the Fc receptors on primary human blood cells was measured in flow cytometry analyses. Frozen PBMCs were thawed, bound native antibodies were stripped from the surfaces and cells were incubated with Fuc− cetuximab, Fuc+ cetuximab or hIgG1 (as negative control) at a concentration of 10 μg/mL. Antibody binding was detected using Cy2-coupled Fab2 fragments directed against human IgG Fab2 fragments, and cells were co-stained with a marker specific for identification of different cell populations. Granulocytes were purified with the Pluriselect-Kit CD15 from fresh blood samples of healthy volunteers and stained accordingly. Analysis of thrombocytes was performed using whole blood and biotinylated antibodies. As result, it was demonstrated that Fuc-cetuximab and Fuc+ cetuximab both strongly bind to monocytes (CD14+) and thrombocytes (CD61+), while only a very low binding to B cells (CD19+) could be observed. However, in contrast to Fuc+ cetuximab, Fuc− cetuximab also strongly binds to granulocytes (CD66+) and NK cells (CD16+CD56+) (FIG. 3). It is believed that binding to the latter blood cell types, in particular to granulocytes (CD66+) and NK cells (CD16+CD56+), may at least in part be responsible for the reduced adverse skin reactions of Fuc− cetuximab when compared to Fuc+ cetuximab. It is remarkable in this respect that a control antibody having an Fc glycosylation pattern similar to that of Fuc− cetugex (including a similarly low fucosylation) showed a much lower granulocyte binding compared to Fuc− cetuximab. Only about 20% of the granulocytes (CD66+) in the blood sample were bound by said control Fuc− antibody while Fuc− cetuximab binds nearly 100% of the granulocytes. Hence, the strong granulocyte binding is a specific feature of the Fuc− cetuximab antibody according to the invention.

### Example 8

### Activation of Granulocytes by the Differently Fucosylated Antibody Variants

To determine the ability of the anti-EGFR antibodies to activate granulocytes, the production of reactive oxygen species and the secretion of lactoferrin were analyzed. Granulocytes form granules, secretion vesicles which contain a mixture of cytotoxic molecules that are released by a process called degranulation following activation of the granulocyte by an immune stimulus. Examples of the cytotoxic molecules contained in the granules are compounds that are involved in the formation of toxic oxygen compounds, lysozyme and lactoferrin. Therefore, an increase in reactive oxygen species and lactoferrin in the supernatant of blood samples indicates the activation of granulocytes. Briefly, a whole blood sample was incubated with Fuc− cetuximab or Fuc+ cetuximab in the presence or absence (control) of target cells expressing EGFR (tumor cell lines A431 (human epidermoid carcinoma cell line of the vulva)). The production of reactive oxygen species is monitored by addition of a dye which reacts to oxidation. The amount of oxidized dye as well as the number of cells producing the reactive oxygen species is determined. Furthermore, the concentration of lactoferrin in the plasma is analyzed using the AssayMax Human Lactoferrin ELISA kit of AssayPro (St. Charles, Mo., USA).

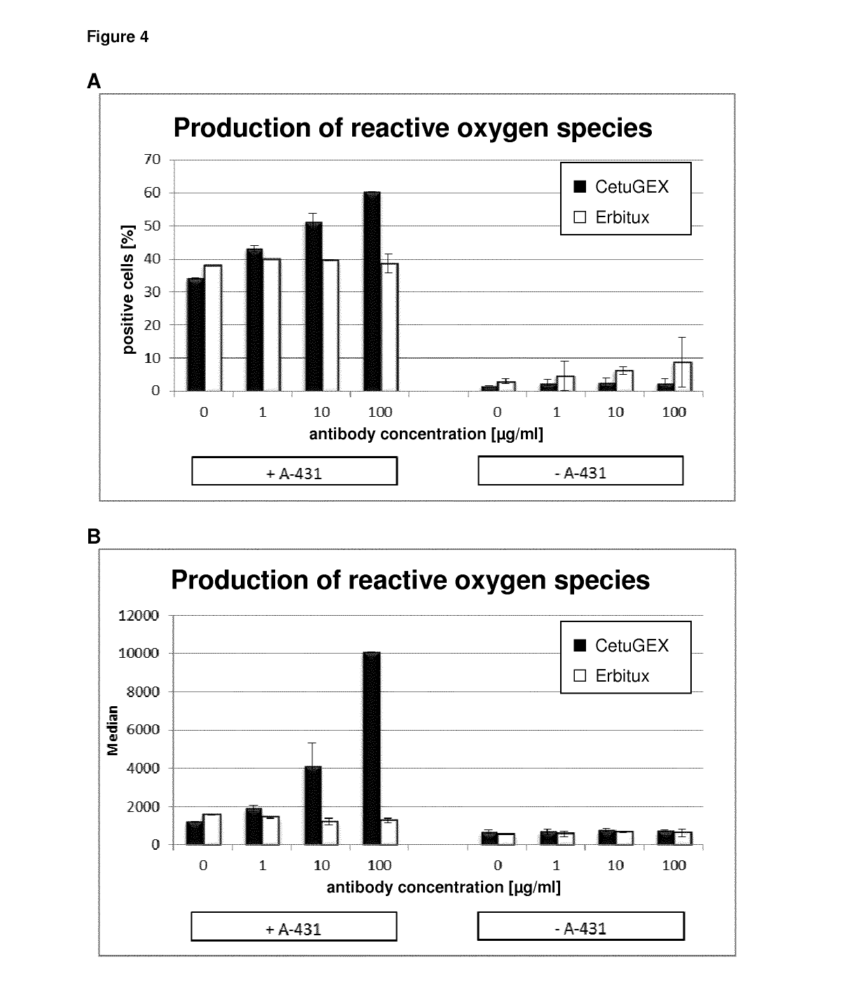

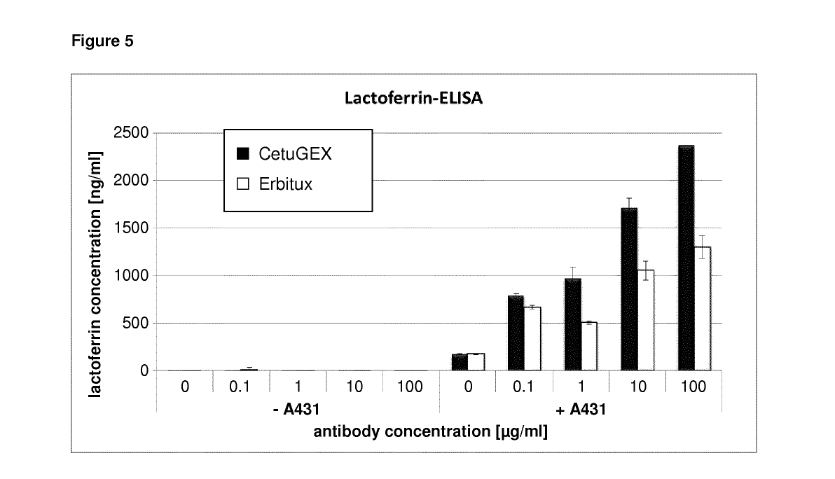

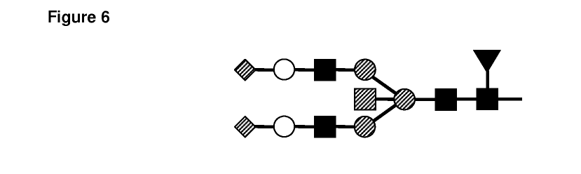

As result, the Fuc− cetuximab according to the present invention shows a significant number of activated granulocytes above the basal activation (0 μg/ml antibody) (FIG. 4A). Most remarkably, the production of reactive oxygen species is about 7-fold increased compared to the control (FIG. 4B). In contrast, Fuc+ cetuximab Fuc+ cetuximab does not show any induction of reactive oxygen species. Furthermore, the Fuc− cetuximab according to the invention also shows an about 2-fold higher induction of lactoferrin release than Fuc+ cetuximab (FIG. 5).

### Additional Indications According to Form POT/ROM 34 for Deposit DSM ACC3078

Applicant herewith requests for those countries which have a respective provision that the furnishing of a sample of the deposited material referred to in the application may only be made to an independent, nominated expert (request of the “expert solution” where applicable, in particular in Australia, Canada, Croatia, Denmark, Finland, Germany, Iceland, Norway, Singapore, Spain, Sweden, United Kingdom, Europe).

For Europe, applicant accordingly requests that a sample of the deposited biological material will be made available as provided in Rule 33(1)(2) EPC until the publication of the mention of the grant of the patent or for 20 years from the date of filing if the application is refused or withdrawn or deemed to be withdrawn, only by the issue of a sample to an expert nominated by the person requesting the sample (Rule 32 EPC).

1. A method of treating an EGFR positive neoplastic disease in a human patient, comprising administering to a patient an anti-EGFR antibody with a glycosylation site in the CH2 domain, wherein 50% or less of the glycans attached to said glycosylation site carry fucose (reduced fucose anti-EGFR antibody) and wherein the reduced fucose anti-EGFR antibody is capable of inducing an antibody-dependent cellular cytotoxicity reaction.

2. The method of claim 1, wherein the reduced fucose anti-EGFR antibody causes one or more of the following:
a) adverse skin reactions of any grade in not more than 75% of the treated patients;
b) adverse skin reactions of grade 3 or higher in not more than 15% of the treated patients;
c) acneiform skin rash in not more than 50% of the treated patients;
d) acneiform skin rash of grade 3 or higher in not more than 15% in treated patients;
e) hypomagnesemia in no more than 15% of treated patients;
f) hypokalemia in no more than 25% of treated patients; and/or
g) diarrhea in no more than 35% of treated patients.

3-20. (canceled)

21. The method of claim 2, wherein the reduced fucose anti-EGFR antibody causes the adverse skin reactions of any grade, the acneiform skin rash, the adverse skin reactions of grade 3 or higher, the acneiform skin rash of grade 3 or higher, the hypomagnesemia and/or the diarrhea in no more than the indicated percentage of the treated patients when administered in an amount of at least 50 mg per dose, and/or when at least 4 or 8 doses where administered, and/or when the doses where administered at least every second week.

22-29. (canceled)

30. The method of claim 1, wherein treatment conditions are used which for at least one other anti-EGFR antibody cause an adverse skin reaction in at least 50% of the patients when using said other anti-EGFR antibody, and/or cause an adverse skin reaction of grade 3 or higher in at least 12% of the patients when using said other anti-EGFR antibody.

31. (canceled)

32. The method of claim 1, wherein the reduced fucose anti-EGFR antibody is administered at a dosage of at least 200 mg per week.

33. The method of claim 1, wherein a) the patient has been previously treated with at least one EGFR inhibitor which caused an adverse reaction of grade 3 or higher, b) the patient is known to have a severe adverse reaction of grade 3 or higher against an EGFR inhibitor which causes such severe adverse reactions, c) the patient has increased risk of developing a severe adverse reaction of grade 3 or higher during treatment with an EGFR inhibitor which causes adverse reactions of grade 3 or higher, d) the patient had a previous treatment which was interrupted, terminated, or wherein the dosage of the EGFR inhibitor had to be reduced because of an adverse reaction against an EGFR inhibitor during treatment, and/or e) the patient has been previously treated with an EGFR inhibitor which is not or cannot be continued because an adverse reaction against the EGFR inhibitor occurred.

34-37. (canceled)

38. The method of claim 33, wherein the EGFR inhibitor is an anti-EGFR antibody selected from the group consisting of cetuximab (Erbitux®), panitumumab (Vectibix®) and GA201; or a tyrosine kinase inhibitor selected from the group consisting of gefitinib, erlotinib and lapatinib.

39-45. (canceled)

46. The method of claim 1, wherein the EGFR positive neoplastic disease is an EGFR positive cancer.

47. The method of claim 46, wherein the EGFR-positive cancer is selected from the group consisting of head and neck cancer, colon cancer, kidney cancer, gastric cancer, esophageal cancer, gallbladder cancer, uterine cancer, breast cancer, rectal cancer, lung cancer, ovarian cancer, and penis cancer.

48. The method of claim 46, wherein the EGFR-positive cancer is selected from the group consisting of colon carcinomas, rectal carcinomas, non-small cell lung carcinomas, squamous cell lung cancer, renal cell carcinomas, triple negative breast cancer, squamous cell carcinomas of the head and neck, esophageal adenocarcinomas, gastric adenocarcinomas, gastroesophageal junction adenocarcinomas, endometrial carcinomas or sarcomas, cervical carcinomas.

49-56. (canceled)

57. The method of claim 1, wherein the EGFR-positive neoplastic disease includes a malignant effusion.

58. The method of claim 57, wherein the malignant effusion is a malignant pleural effusion or a malignant peritoneal effusion.

59-60. (canceled)

61. A method for treating kidney cancer in a human patient, comprising administering to a patient an anti-EGFR antibody with a glycosylation site in the CH2 domain, wherein 50% or less of the glycans attached to said glycosylation site carry fucose (reduced fucose anti-EGFR antibody) and wherein the reduced fucose anti-EGFR antibody is capable of inducing an antibody-dependent cellular cytotoxicity reaction.

62-64. (canceled)

65. The method of claim 61, wherein the kidney cancer is selected from clear cell renal cell carcinoma, papillary renal cell carcinoma, and metastasizing kidney cancer.

66. (canceled)

67. A method for treating malignant effusion in a human patient having an EGFR positive neoplastic disease, comprising administering to a patient an anti-EGFR antibody with a glycosylation site in the CH2 domain, wherein 50% or less of the glycans attached to said glycosylation site carry fucose (reduced fucose anti-EGFR antibody) and wherein the reduced fucose anti-EGFR antibody is capable of inducing an antibody-dependent cellular cytotoxicity reaction.

68-70. (canceled)

71. The method of claim 67, wherein the effusion is a pleural effusion and the EGFR positive neoplastic disease is selected from the group consisting of breast cancer, lung cancer, gastric cancer and esophageal cancer.

72-73. (canceled)

74. The method of claim 67, wherein the effusion is a peritoneal effusion and the EGFR positive neoplastic disease is selected from the group consisting of pancreatic cancer, ovarian cancer, gastric cancer, esophageal cancer and colon cancer.

75-80. (canceled)

81. The method of claim 1, wherein the reduced fucose anti-EGFR antibody has an amount of fucose in the carbohydrate chains attached to the CH2 domain which is in the range of from 3% to 20%.

82. The method of claim 1, wherein the reduced fucose anti-EGFR antibody comprises one or more of the following glycosylation characteristics in the glycosylation site of the CH2 domain:
(i) a relative amount of glycans carrying a bisecting GlcNAc of at least 5%;
(ii) a relative amount of glycans carrying at least one galactose of at least 50%;
(iii) a relative amount of glycans carrying two galactoses of at least 10%;
(iv) a relative amount of glycans carrying at least one sialic acid;
(v) a relative amount of glycans carrying two sialic acids, in particular NeuAc, of at least 0.5%;
(vi) it does not comprise NeuGc;
(vii) it does not comprise Galα1,3-Gal; and
(viii) it comprises α2,6-coupled NeuAc.

83. (canceled)

84. The method of claim 1, wherein the reduced fucose anti-EGFR antibody comprises an additional glycosylation site in the heavy chain variable region VH; and comprises one or more of the following glycosylation characteristics in the glycosylation site of the VH domain:
(i) a relative amount of glycans carrying a fucose residue of 40% or less;
(ii) a relative amount of glycans carrying a bisecting GlcNAc of at least 35%;
(iii) a relative amount of glycans carrying at least one galactose of at least 85%;
(iv) a relative amount of glycans carrying at least two galactoses of at least 70%;
(v) a relative amount of glycans carrying at least one sialic acid of at least 50%; and
(vi) a relative amount of glycans carrying at least two sialic acids of at least 35%.

85-90. (canceled)

91. The method of claim 1, wherein the anti-EGFR antibody comprises a heavy chain variable region comprising (a) a CDR1 having the amino acid sequence of SEQ ID NO: 1, a CDR2 having the amino acid sequence of SEQ ID NO: 2, and a CDR3 having the amino acid sequence of SEQ ID NO: 3; and/or (b) the amino acid sequence of SEQ ID NO: 7 or 9, or an amino acid sequence which is at least 80% identical thereto.

92. (canceled)

93. The method of claim 1, wherein the anti-EGFR antibody comprises a light chain variable region comprising (a) a CDR1 having the amino acid sequence of SEQ ID NO: 4, a CDR2 having the amino acid sequence of SEQ ID NO: 5, and a CDR3 having the amino acid sequence of SEQ ID NO: 6; and/or (b) the amino acid sequence of SEQ ID NO: 8 or 10, or an amino acid sequence which is at least 80% identical thereto.

94-108. (canceled)

109. The method of claim 1, wherein the anti-EGFR antibody is conjugated to a further agent.

110-111. (canceled)

112. The method of claim 1, wherein the patient is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F).

113. (canceled)

114. The method of claim 1, wherein the reduced fucose anti-EGFR antibody is for treatment of patients irrespective of their FcγRIIIa allotype.

115. The method of claim 1, wherein a) the EGFR positive neoplastic disease comprises a K-RAS mutation, b) the reduced fucose anti-EGFR antibody is for treatment of patients irrespective of the K-RAS mutational status of the EGFR positive neoplastic disease, and/or c) the EGFR positive neoplastic disease is resistant to or has progressed after treatment with at least one or more anti-cancer therapies.

116-126. (canceled)

127. The method of claim 1, wherein the anti-EGFR antibody is used in combination with
(i) at least one chemotherapeutic agent; and/or
(ii) at least one further therapeutic antibody which is different from the reduced fucose anti-EGFR antibody; and/or
(iii) cancer surgery and/or radiotherapy.

128-133. (canceled)

134. The method of claim 1, wherein the treatment includes the administration of the reduced fucose anti-EGFR antibody in an amount of a) from 12 to 2000 mg per dose, b) 240 to 1200 mg per dose, or c) 250 to 1500 mg per dose.

135-136. (canceled)

137. The method of claim 1, wherein the treatment includes the administration of the reduced fucose anti-EGFR antibody in an amount of a) from 0.5 to 50 mg/kg body weight of the patient per dose, b) from 2 to 20 mg/kg body weight of the patient per dose, c) from 10 to 25 mg/kg body weight of the patient per dose, d) from 5 to 1000 mg/m² body weight of the patient per dose, e) from 100 to 600 mg/m² body weight of the patient per dose or f) from 300 to 750 mg/m² body weight of the patient per dose.

138-142. (canceled)

143. The method of claim 1, wherein the treatment includes the administration of one dose of the reduced fucose anti-EGFR antibody every 5 days or less frequently.

144-164. (canceled)

165. A method of inducing a granulocyte-driven and/or macrophage-driven immune reaction against cells of an EGFR positive neoplastic disease in a human patient, wherein the treatment comprises administering to the patient an anti-EGFR antibody with a glycosylation site in the CH2 domain, wherein 50% or less of the glycans attached to said glycosylation site carry fucose (reduced fucose anti-EGFR antibody) and wherein the reduced fucose anti-EGFR antibody is capable of inducing an antibody-dependent cellular cytotoxicity reaction.

166-172. (canceled)

173. A method for reducing the adverse reactions in a treatment with an EGFR inhibitor of a patient having an EGFR positive neoplastic disease, comprising the step of treating the patient with an anti-EGFR antibody with a glycosylation site in the CH2 domain, wherein 50% or less of the glycans attached to said glycosylation site carry fucose (reduced fucose anti-EGFR antibody) and wherein the reduced fucose anti-EGFR antibody is capable of inducing an antibody-dependent cellular cytotoxicity reaction.

174-208. (canceled)


SEQUENCE LISTING (10 sequences)

SEQ ID NO: 1 - protein, 5 aa, Artificial Sequence
  SOURCE 1..5: /mol_type=“protein” /note=“CDRH1” /organism=“Artificial Sequence”
NYGVH

SEQ ID NO: 2 - protein, 16 aa, Artificial Sequence
  SOURCE 1..16: /mol_type=“protein” /note=“CDRH2” /organism=“Artificial Sequence”
VIWSGGNTDYNTPFTS

SEQ ID NO: 3 - protein, 11 aa, Artificial Sequence
  SOURCE 1..11: /mol_type=“protein” /note=“CDRH3” /organism=“Artificial Sequence”
ALTYYDYEFAY

SEQ ID NO: 4 - protein, 11 aa, Artificial Sequence
  SOURCE 1..11: /mol_type=“protein” /note=“CDRL1” /organism=“Artificial Sequence”
RASQSIGTNIH

SEQ ID NO: 5 - protein, 7 aa, Artificial Sequence
  SOURCE 1..7: /mol_type=“protein” /note=“CDRL2” /organism=“Artificial Sequence”
YASESIS

SEQ ID NO: 6 - protein, 9 aa, Artificial Sequence
  SOURCE 1..9: /mol_type=“protein” /note=“CDRL3” /organism=“Artificial Sequence”
QQNNNWPTT

SEQ ID NO: 7 - protein, 119 aa, Artificial Sequence
  SOURCE 1..119: /mol_type=“protein” /note=“humanized heavy chain variable region” /organism=“Artificial Sequence”
QVQLQESGPGLVKPSETLSLTCTVSGFSISNYGVHWIRQSPGKGLEWIGVIWSGGNTDYNTPFTSRVTISKDNSKNQVSLKLSSVTAADTAVYYCARALTYYDYEFAYWGQGTLVTVSA

SEQ ID NO: 8 - protein, 108 aa, Artificial Sequence
  SOURCE 1..108: /mol_type=“protein” /note=“humanized light chain variable region” /organism=“Artificial Sequence”
EIVLTQSPDFQSVTPKEKVTITCRASQSIGTNIHWYQQKPDQSPKLLIKYASESISGVPSRFSGSGSGTDFTLTINSLEAEDAATYYCQQNNNWPTTFGAGTKLEIKR

SEQ ID NO: 9 - protein, 119 aa, artificial sequences
  SOURCE 1..119: /mol_type=“protein” /note=“mouse heavy chain variable region” /organism=“artificial sequences”
QVQLKQSGPGLVQPSQSLSITCTVSGFSLTNYGVHWIRQPPGKGLEWIGVIWSGGNTDYNTPFTSRLSINKDNSKSQVFFKMNSLQSNDTAIYYCARALTYYDYEFAYWGQGTLVTVSA

SEQ ID NO: 10 - protein, 108 aa, artificial sequences
  SOURCE 1..108: /mol_type=“protein” /note=“mouse light chain variable region” /organism=“artificial sequences”
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR